(19) United States
(12) Patent Application Publication
(10) Pub. No.: US 2010/0255037 A1
(43) Pub. Date: Oct. 7, 2010

(54) NOVEL SEQUENCES OF HAEMONCHUS CONTORTUS, IMMUNOGENIC COMPOSITIONS, METHODS FOR PREPARATION AND USE THEREOF

(76) Inventors: Jennifer Louise Sexton, Victoria (AU); Dadna Hartman, Victoria (AU); Ben Cocks, Victoria (AU)

Correspondence Address: COOPER & DUNHAM, LLP, 30 Rockefeller Plaza, 20th Floor, NEW YORK, NY, 10112, US

(21) Appl. No.: 12/312,481

(22) PCT Filed: Nov. 15, 2007
(86) PCT No.: PCT/EP2007/062360

§ 371 Date: Jan. 19, 2010

(30) Foreign Application Priority Data
Nov. 17, 2006 (AU) 2006906429

(51) Int. Cl. A61K 39/00; C07H 21/04; C07K 14/00; C12N 15/63; C12N 5/10; C12N 1/21; C07K 16/00

(52) U.S. Cl. 424/265.1; 536/23.7; 530/324; 435/320.1; 435/349; 435/365; 435/358; 435/354; 435/367; 435/252.3; 435/252.32; 435/252.33; 435/252.31; 435/252.34; 530/387.9

(57) ABSTRACT

The present invention relates to novel sequences of H. contortus and the proteins encoded therein. This invention also relates to immunogenic compositions, methods for their preparation and the diagnostic, prophylactic or therapeutic use of these sequences and the proteins encoded therein.

### FIELD OF INVENTION

The present invention relates to novel sequences of Haemonchus contortus and the proteins encoded therein. This invention further relates to immunogenic compositions, methods for their preparation and the diagnostic, prophylactic or therapeutic use of these sequences and the proteins encoded therein.

### BACKGROUND OF THE INVENTION

H. contortus also known as Barber's pole worm, stomach worm or wire worm is a blood-feeding nematode that infects the lining of the gastrointestinal tract and abomasum of ruminant animals. Infection is also possible through the skin. Haemonchus requires no immediate host but may live in soil and water of pastures where the ruminant animals are grazing.

The economic loss from the effects of reduction in weight, loss of production and agalactia through to death of domestic animals infected by Haemonchus is considerable. In Australia, for example, it is estimated that approximately one third of all sheep are likely to be infected with Haemonchus.

Anthelmintic chemicals are typically used to treat domestic animals infected by H. contortus but there is an increased frequency of documented resistance to these products. In addition, it is common for an animal to be infected with several species of trichostronglyids at the same time. Therefore, there is a growing need to have alternative ways of preventing and treating animals infested with H. contortus as well as other trichostronglyids.

The development of a vaccine against H. contortus would overcome many of the drawbacks inherent in chemical treatment. The protection would be longer lasting and the animal could be individually targeted thus avoiding the problems of toxicity associated with chemical treatment.

Nevertheless, it is particularly difficult to develop vaccines against parasitic helminth infections because of the complexity of the parasite's life cycle. Although vaccine candidates have been reported for H. contortus (see: WO88/00835), there is a need to develop further and more efficacious vaccines, particularly as recombinant vaccines.

### SUMMARY OF THE INVENTION

The invention relates to novel sequences of H. contortus and proteins which are encoded by those sequences. It further relates to the use of these sequences and proteins of the invention for diagnostic, prophylactic or therapeutic purposes.

In one aspect of the invention, isolated polynucleotide sequences encoding a H. contortus polypeptide are provided with nucleotide sequence of: SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. 25 (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2).

In another aspect of the invention, isolated polynucleotide sequences which are fragments of SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2) are provided.

In a further aspect of the invention, homologous polynucleotide sequences which are at least 80% homologous, preferably 90%, more preferably 95% and most preferably 99% homology to the sequences of SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. 25 (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2) are provided.

In yet another aspect of the invention, polynucleotide sequences which hybridize under high stringency conditions to an isolated sequence of SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. 25 (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2) are provided.

In a second aspect of the invention, polypeptide sequences of H. contortus are provided: SEQ ID No. 6 (FIG. 1-2), SEQ ID No. 7 (FIG. 1-5), SEQ ID No. 9 (FIG. 2-2), SEQ ID No. 10 (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. 30 (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5).

In one aspect of the invention, fragments of polypeptide sequences SEQ ID No. 6 (FIG. 1-2), SEQ ID No. 7 (FIG. 1-5), SEQ ID No. 9 (FIG. 2-2), SEQ ID No. 10 (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. 30 (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5) are provided.

In another aspect of the invention, homologous polypeptides sequences are provided wherein said sequence is at least 80% homologous, preferably 90%, more preferably 95% and most preferably 99% homology to SEQ ID No. 6 (FIG. 1-2), SEQ ID No. 7 (FIG. 1-5), SEQ ID No. 9 (FIG. 2-2), SEQ ID No. (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5).

In a third aspect of the invention, expression vectors are provided, comprising a polynucleotide sequence of the invention operably linked to a control sequence which is capable of providing for the expression of the polynucleotide sequence by a host cell.

In a fourth of the invention, host cells are provided comprising a polypeptide sequence of the invention.

In a fifth aspect of the invention, an antibody which binds to a protein which has a polypeptide sequence of the invention is provided.

In a sixth aspect of the invention, the use of a polypeptide sequence of the invention encoding a H. contortus protein for the manufacturing of an immunogenic composition for prophylaxis or treatment of H. contortus infection is provided.

In a seventh aspect of the invention, an immunogenic composition for the prophylaxis or treatment of H. contortus infection comprising a polypeptide sequence of the invention encoding a H. contortus protein is provided.

In an eighth aspect of the invention, an immunogenic composition comprising at least one additional immunogenic sequence derived from another trichostrongylidae other than Haemonchus is provided.

In a ninth aspect of the invention, methods for the preparation of an immunogenic composition of the invention are provided.

In a tenth aspect of the invention, a diagnostic kit for the detection of antibodies against H. contortus is provided.

In one aspect of the invention, the diagnostic kit comprises antibodies against a protein with a polypeptide sequence of the invention from H. contortus.

In an eleventh aspect of the invention, use of a polynucleotide sequence of the invention for the manufacturing of an immunogenic composition for prophylaxis or treatment of H. contortus infection is provided.

In a twelfth aspect of the invention, an immunogenic composition for the prophylaxis or treatment of H. contortus infection comprising a polynucleotide sequence of the invention is provided.

In one aspect, the immunogenic composition comprises at least one additional sequence derived from another trichostronglyid other than H. contortus.

### DETAILED DESCRIPTION OF THE INVENTION

Nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated polynucleotides” what is intended is a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically. It is possible for an isolated polynucleotide to exist but not qualify as a purified polynucleotide.

In addition, isolated nucleic acid molecules of the invention include DNA molecules which comprise a sequence substantially different from those described above but which, due to the degeneracy of the genetic code, still encode a H. contortus polypeptides and peptides of the present invention. This includes the genetic code and species-specific codon preferences known in the art. Thus, it would be routine for one skilled in the art to generate the degenerate variants described above, for instance, to optimize codon expression for a particular host (e.g., change codons in the bacteria mRNA to those preferred by a mammalian or other bacterial host such as Escherichia coli).

The invention provides isolated nucleic acid molecules having the nucleotide sequence shown in Table 1 or a nucleic acid molecule having a sequence complementary to one of the above described sequences. Such isolated molecules, particularly DNA molecules, are useful as probes for gene mapping and for identifying H. contortus in a biological sample, for instance, by PCR, Southern blot, Northern blot, or other form of hybridization analysis.

The present invention is further directed to nucleic acid molecules encoding fragments of the nucleotide sequences described herein. Further, the invention includes polynucleotides comprising fragments specified by size, in nucleotides, rather than by nucleotide positions. Such nucleotide fragments may be useful as diagnostic probes and primers.

TABLE 1
Sequences

| Sequence | | |
|---|---|---|
| Identi- | | |
| fier | Figure | Description |
| SEQ ID | | His-linker Forward |
| NO. 1 | | GTCCCACCATCACCATCACCATACCATGGG |
| SEQ ID | | His-linker Reverse |
| NO. 2 | | AATTCCCATGGTATGGTGATGGTGATGGTGG |
| SEQ ID | | T7 Primer |
| NO. 3 | | GTAATACGACTCACTATAG |
| SEQ ID | | SP6 Primer |
| NO. 4 | | ATTTAGGTGACACTATAG |
| SEQ ID | FIG. | Nucleotide sequence of clone |
| NO. 5 | 1-1 | S7T104D12, 573 base pairs |
| | FIG. | Nucleotide sequence of clone |
| | 1-2 | S7T104D12 |
| SEQ ID | FIG. | Amino acid sequence of clone |
| NO. 6 | 1-2 | S7T104D12, ORF of 144 amino acids |
| SEQ ID | FIG. | Amino acid sequence for S7T104D12 E. |
| NO. 7 | 1-5 | coli expression clone, 161 amino |
| | | acids including initiation Met, His |
| | | tag and linker residues, and 139 |
| | | amino acids of S7T104D12 without N- |
| | | terminal residues |
| SEQ ID | FIG. | Nucleotide sequence of clone |
| NO. 8 | 2-1 | S4T58F9, 977 base pairs |
| | FIG. | Nucleotide sequence of clone S4T58F9 |
| | 2-2 | |
| SEQ ID | FIG. | Amino acid sequence of clone |
| NO. 9 | 2-2 | S4T58F9, ORF of 302 amino acids |
| SEQ ID | FIG. | Amino acid sequence for S4T58F9 E. |
| NO. 10 | 2-5 | coli expression clone, 287 amino |
| | | acids including initiation Met, His |
| | | tag and linker residues, and 265 |
| | | amino acids of S4T58F9 without N- |
| | | terminal residues |
| SEQ ID | | T3 primer |
| NO. 11 | | CAATTAACCCTCACTAAAG |
| SEQ ID | FIG. | Nucleotide sequence of clone S1-381, |
| NO. 12 | 3-1 | 846 base pairs |
| | FIG. | Nucleotide sequence of clone S1-381 |
| | 3-2 | |
| SEQ ID | FIG. | Amino acid sequence of clone S1-381, |
| NO. 13 | 3-2 | ORF of 167 amino acids |
| SEQ ID | FIG. | Amino acid sequence for S1-381 E. |
| NO. 14 | 3-5 | coli expression clone, 168 amino |
| | | acids including initiation Met, His |
| | | tag and linker residues, and 146 |
| | | amino acids of S1-381 without N- |
| | | terminal residues |
| SEQ ID | | λTriplex 5′ Primer |
| NO. 15 | | TCCGAGATCTGGACGAGC |
| SEQ ID | FIG. | Nucleotide sequence of clone G0142, |
| NO. 16 | 4-1 | 1788 base pair |
| | FIG. | Nucleotide sequence of clone G0142 |
| | 4-2 | |
| SEQ ID | FIG. | Amino acid sequence of clone G0142, |
| NO. 17 | 4-2 | ORF of 290 amino acids |
| SEQ ID | FIG. | Amino acid sequence for G0142 E. |
| NO. 18 | 4-5 | coli expression clone, 312 amino |
| | | acids including initiation Met, His |
| | | tag and linker residues, and 290 |
| | | amino acids of G0142 |
| SEQ ID | FIG. | Nucleotide sequence of clone S1T1F1, |
| NO. 19 | 5-1 | 881 base pairs |
| | FIG. | Nucleotide sequence of clone S1T1F1 |
| | 5-2 | |
| SEQ ID | FIG. | Amino acid sequence of clone S1T1F1, |
| NO. 20 | 5-2 | ORF of 228 amino acids |
| SEQ ID | FIG. | Amino acid sequence for S1T1F1 E. |
| NO. 21 | 5-5 | coli expression clone, 218 amino |
| | | acids including initiation Met, His |
| | | tag and linker residues, and 196 |
| | | amino acids of S1T1F1 without N- |
| | | terminal residues |
| SEQ ID | FIG. | Nucleotide sequence of clone S2- |
| NO. 22 | 6-1 | 259MF, 1609 base pair |
| | FIG. | Nucleotide sequence of clone S2- |
| | 6-2 | 259MF |
| SEQ ID | FIG. | Amino acid sequence of clone S2- |
| NO. 23 | 6-2 | 259MF, ORF of 502 amino acids |
| SEQ ID | FIG. | Amino acid sequence for S2-259MF |
| NO. 24 | 6-5 | Baculovirus expression clone, 594 |
| | | amino acids including initiation |
| | | Met, secretion signal, His tag and |
| | | an additional 40 amino acids at the |
| | | C-terminus due to unusual recombi- |
| | | nation during cloning, and 491 amino |
| | | acids of S2-259 missing 11 amino |
| | | acids from C-terminus due to unusual |
| | | recombination event |
| SEQ ID | FIG. | Nucleotide sequence of clone G1083P, |
| NO. 25 | 7-1 | 1200 base pairs |
| | FIG. | Nucleotide sequence of clone G1083P |
| | 7-2 | |
| SEQ ID | FIG. | Amino acid sequence of clone G1083P, |
| NO. 26 | 7-2 | ORF of 289 amino acids |
| SEQ ID | FIG. | Amino acid sequence for G1083P E. |
| NO. 27 | 7-5 | coli expression clone, 252 amino |
| | | acids including initiation Met, His |
| | | tag and additional linker residues, |
| | | and 230 amino acids from G1083P |
| | | minus C-terminal transmembrane |
| | | domain |
| SEQ ID | FIG. | Nucleotide sequence of clone dd165- |
| NO. 28 | 8-1 | 2NTC#1, 579 base pairs |
| | FIG. | Nucleotide sequence of clone dd165- |
| | 8-2 | 2NTC#1 |
| SEQ ID | FIG. | Amino acid sequence of clone dd165- |
| NO. 29 | 8-2 | 2NTC#1, ORF of 122 amino acids |
| SEQ ID | FIG. | Amino acid sequence for dd165-2NTC#1 |
| NO. 30 | 8-5 | E. coli expression clone, 144 amino |
| | | acids including initiation Met, His |
| | | tag and additional linker residues, |
| | | and 122 amino acids of dd165-2NTC#1 |
| SEQ ID | FIG. | Nucleotide sequence of clone |
| NO. 31 | 9-1 | S4T69C3, 1383 base pairs |
| | FIG. | Nucleotide sequence of clone S4T69C3 |
| | 9-2 | |
| SEQ ID | FIG. | Amino acid sequence of clone |
| NO. 32 | 9-2 | S4T69C3, ORF of 434 amino acids |
| SEQ ID | FIG. | Amino acid sequence for S4T69C3 E. |
| NO. 33 | 9-5 | coli expression clone, 441 amino |
| | | acids including initiation Met, His |
| | | tag and additional linker, and 419 |
| | | amino acids of S4T69C3 minus the |
| | | signal sequence |
| SEQ ID | FIG. | Nucleotide sequence of clone YAd189, |
| NO. 34 | 10-1 | approximately 1500 base pairs |
| | FIG. | Nucleotide sequence of clone YAd189 |
| | 10-2 | |
| SEQ ID | FIG. | Amino acid sequence of clone YAd189, |
| NO. 35 | 10-2 | ORF of 421 amino acids |
| SEQ ID | FIG. | Amino acid sequence for YAd189 E. |
| NO. 36 | 10-5 | coli expression clone, 443 amino |
| | | acids including initiation Met, His |
| | | tag and additional linker, and 421 |
| | | amino acids of YAd189 |
| SEQ ID | FIG. | Nucleotide sequence of clone YAd219, |
| NO. 37 | 11-1 | 464 base pairs |
| | FIG. | Nucleotide sequence of clone YAd219 |
| | 11-2 | |
| SEQ ID | FIG. | Amino acid sequence of clone YAd219, |
| NO. 38 | 11-2 | ORF of 97 amino acids |
| SEQ ID | FIG. | Amino acid sequence for YAd219 E. |
| NO. 39 | 11-5 | coli expression clone, 94 amino |
| | | acids including initiation Met, His |
| | | tag and additional linker, and 72 |
| | | amino acid of YAd219 minus the |
| | | signal sequence |
| SEQ ID | FIG. | Nucleotide sequence of clone |
| NO. 40 | 12-1 | S4T55C6, 2036 base pairs |
| | FIG. | Nucleotide sequence of clone S4T55C6 |
| | 12-2 | |
| SEQ ID | FIG. | Amino acid sequence of clone |
| NO. 41 | 12-2 | S4T55C6, ORF of 320 amino acids |
| SEQ ID | FIG. | Amino acid sequence for S4T55C6 E. |
| NO. 42 | 12-5 | coli expression clone, 312 amino |
| | | acids including the initiation Met, |
| | | His tag and additional linker |
| | | residues, and 290 amino acids of |
| | | S4T55C6 minus signal sequence |

In one aspect of the invention, preferred fragments are the open reading frame (ORF) sequences from proteins of H. contortus.

The sequences which are open reading frame (ORF) sequences encoding proteins of H. contortus are selected from the group consisting of SEQ ID No. 6 (FIG. 1-2), SEQ ID No. 7 (FIG. 1-5), SEQ ID No. 9 (FIG. 2-2), SEQ ID No. 10 (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. 30 (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5)

In a preferred aspect of the invention such ORFs encode immunogenic polypeptides useful against H. contortus infection.

By “immunogenic”, it is meant that there is a protective effect in the target animal which includes prevention of helminth infection, viability and/or fecundity as well as reduced infection, growth, viability and/or fecundity and also includes various levels of amelioration of symptoms of helminth infection. Even a partial reduction in helminth numbers or ability to spread is useful in controlling helminth infection in animals. The protective effect can also be measured by increased productivity of a group of animals.

Although a polypeptide representing a complete ORF may be the closest approximation of a protein native to an organism, it is not always preferred to express a complete ORF in a heterologous system. It may be challenging to express and purify a highly hydrophobic protein by common laboratory methods. Some of the immunogenic compositions described herein may be modified slightly to simplify the production of recombinant protein. For example, nucleotide sequences which encode highly hydrophobic domains, such as those found at the amino terminal signal sequence, may be excluded from some constructs used for in vitro expression of the polypeptides. Furthermore, any highly hydrophobic amino acid sequences occurring at the carboxy terminus have also been excluded from the recombinant expression constructs. Thus, in another aspect of the invention, a polypeptide which represents a truncated or modified ORF of the ORFs disclosed above may be used in an immunogenic composition.

In another aspect, the invention provides isolated nucleic acid molecules comprising polynucleotides which hybridize under stringent hybridization conditions to a portion of a polynucleotide in a nucleic acid molecule of the invention.

In one aspect of the invention, the polynucleotides hybridize under stringent hybridization conditions to an isolated polynucleotide sequence coding a H. contortus polypeptide of the invention.

By “stringent hybridization” conditions it is intended overnight incubation at 42 C in a solution comprising: 50% formamide, 5×SSC (150 mM NaCl, 15 mM trisodium citrate), 50 mM sodium phosphate (pH 7; 6), 5×Denhardt's solution, 10% dextran sulfate, and 20 g/m denatured, sheared salmon sperm DNA, followed by washing the filters in 0.1×SSC at about 65 C.

By polynucleotides which hybridize it is intended that the polynucleotides (either DNA or RNA) which hybridize are at least about 15 nucleotides, and more preferably at least about 20 nucleotides, still more preferably at least about 30 nucleotides, and even more preferably about 30-70 nucleotides of the reference polynucleotide. These are useful as diagnostic probes and primers.

As noted above, such portions are useful diagnostically either as probes according to conventional DNA hybridization techniques or as primers for amplification of a target sequence by PCR as described, for instance, in Molecular Cloning, A Laboratory Manual, 2nd. edition, Sambrook, J., Fritsch, E. F. and Maniatis, T., eds., Cold Spring Harbor Laboratory Press, Cold Spring Harbor, N.Y. (1989), the entire disclosure of which is hereby incorporated herein by reference. Since nucleic acid sequences encoding the polypeptides of the present invention are provided generating polynucleotides which hybridize to portions of these sequences would be routine to the skilled artisan

The present invention further relates to “substantially homologous sequences” or “homologous sequences” of the present invention, which encode portions, analogs or derivatives of the H. contortus polypeptides. Such variants include those produced by nucleotide substitutions, deletions or additions. The substitutions, deletions or additions may involve one or more nucleotides. These variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the H. contortus polypeptides disclosed herein or portions thereof. Also especially preferred in this regard are conservative substitutions.

The present application is further directed to polynucleotides sequences at least 80% homologous to a nucleic acid sequences disclosed herein. Embodiments of the invention comprise a polynucleotide having a nucleotide sequence at least 80% homologous, more preferably at least 90%, and most preferably at least 99% identical to a nucleotide sequence encoding any of the amino acid sequences of the full-length polypeptides and a nucleotide sequence complementary to any of the nucleotide sequences described above and in Table 1 and FIGS. 1 to 12.

By a polynucleotide having a nucleotide sequence at least, for example, 95% to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the H. contortus polypeptide.

In one embodiment of the invention, the isolated polynucleotide sequence is at least 80% homologous, preferably 90% homologous, more preferably 95% and most preferably 99% homologous to the H. contortus sequences described in Table 1 and FIGS. 1 to 12.

The present invention also relates to expression vectors which include the isolated polynucleotide sequences of the present invention, host cells which are genetically engineered with the recombinant vectors, and the production of H. contortus polypeptides or fragments thereof by recombinant techniques.

In one aspect of the invention, an expression vector comprising a polynucleotide sequence of the invention as described in Table 1 and FIGS. 1-12 is provided, operably linked to a control sequence which is capable of providing expression of the polynucleotide sequence by a host cell.

Recombinant constructs may be introduced into host cells using well known techniques such as infection, transduction, transfection transvection, electroporation and transformation. The vector may be for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

Expression vectors useful in the present invention include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

Preferred expression vectors of the present invention are E. coli and Baculovirus Gateway. Other suitable vectors will be readily apparent to the skilled artisan.

Nucleic acid sequences of the present invention can be operatively linked to expression vectors containing regulatory sequences such as promoters, operators, repressors, enhancers, termination sequences, origins of replication, and other regulatory sequences that are compatible with the host cell and that control the expression of the nucleic acid sequences. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography and high performance liquid chromatography is employed for purification.

Preferably, the polypeptides of the present invention are affinity purified following solubilisation of the inclusion bodies with urea.

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells.

In one aspect of the invention, an isolated polypeptide sequence of H. contortus with the amino acid sequence of:
(a) SEQ ID No. 6 (FIG. 1-2), SEQ ID No. 7 (FIG. 1-5), SEQ ID No. 9 (FIG. 2-2), SEQ ID No. 10 (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5) is provided.

By “isolated polypeptide”, it is meant that the polypeptide prepared from recombinant DNA or RNA, or of synthetic origin or natural origin, or some combination thereof, which is not associated with proteins that it is normally found with in nature, is separated from the cell in which it normally occurs, is free of other proteins from the same cellular source, is expressed by a cell from a different species, or does not occur in nature. It is possible for an isolated polypeptide to exist but not qualify as a purified polypeptide.

To improve or alter the characteristics of H. contortus polypeptides of the present invention, protein engineering may be employed. Recombinant DNA technology known to those skilled in the art can be used to create novel mutant proteins or muteins including single or multiple amino acid substitutions, deletions, additions, or fusion proteins. Such modified polypeptides can show, e.g., enhanced activity or increased stability. In addition, they may be purified in higher yields and show better solubility than the corresponding natural polypeptide, at least under certain purification and storage conditions.

The present invention is further directed to polynucleotide encoding portions or fragments of the amino acid sequences described herein as well as to portions or fragments of the isolated amino acid sequences described in Table 1 and FIGS. 1 to 12.

The present invention further includes variations of the H. contortus. Such mutants include deletions, insertions, inversions, repeats, and type substitutions selected according to general rules known in the art so as to have little effect on immunogenic activity tolerant of amino acid substitutions.

The polypeptides of the present invention also include “substantially homologous polypeptides” having an amino acid sequence at least 80% homologous, more preferably at least 90% homologous, and most preferably 99% homologous to those described in the invention.

As described below, the polypeptides of the present invention can also be used to raise polyclonal and monoclonal antibodies, which are useful in assays for detecting H. contortus protein expression or as agonists and antagonists capable of enhancing or inhibiting H. contortus protein function.

Immunogenic polypeptides of the invention are therefore useful to raise antibodies, including monoclonal antibodies that bind specifically to a polypeptide of the invention. Immunogenic polypeptides of the invention preferably contain a sequence of at least seven, more preferably at least nine and most preferably between about 10 to about 50 amino acids (i.e. any integer between 7 and 50) contained within the amino acid sequence of a polypeptide of the invention. However, peptides or polypeptides comprising a larger portion of an amino acid sequence of a polypeptide of the invention, containing about 50 to about 100 amino acids, or any length up to and including the entire amino acid sequence of a polypeptide of the invention.

H. contortus protein-specific antibodies for use in the present invention can be raised against the intact H. contortus protein of the invention or an immunogenic polypeptide fragment thereof.

As used herein, the term “antibody” is meant to include intact molecules, single chain whole antibodies, and antibody fragments. Also included in the present invention are chimeric and humanized monoclonal antibodies and polyclonal antibodies specific for the polypeptides of the present invention.

The antibodies of the present invention may be prepared by any of a variety of methods. For example, cells expressing a polypeptide of the present invention or an antigenic fragment thereof can be administered to an animal in order to induce the production of sera containing polyclonal antibodies. For example, a preparation of H. contortus polypeptide or fragment thereof is prepared and purified to render it substantially free of natural contaminants. Such a preparation is then introduced into an animal in order to produce polyclonal antisera of greater specific activity.

Antibodies and fragments thereof of the present invention may be described by the portion of a polypeptide of the present invention recognized or specifically bound by the antibody. Antibody binding fragments of a polypeptide of the present invention may be described or specified in the same manner as for polypeptide fragments discussed above. i.e. by N-terminal and C-terminal positions or by size in contiguous amino acid residues. Any number of antibody binding fragments, of a polypeptide of the present invention, specified by N-terminal and C-terminal positions or by size in amino acid residues, as described above, may also be excluded from the present invention. Therefore, the present invention includes antibodies the specifically bind a particularly described fragment of a polypeptide of the present invention and allows for the exclusion of the same.

As one of skill in the art will appreciate, the polypeptides of the present invention and the immunogenic fragments thereof described above can be combined with parts of the constant domain of immunoglobulins (IgG), resulting in chimeric polypeptides. These fusion proteins facilitate purification and show an increased half-life in vivo.

Antibodies of the present invention have a variety of potential uses that are within the scope of the present invention. For example, such antibodies can be used (a) as vaccines to passively immunize an animal in order to protect the animal from H. contortus infections, (b) as reagents in assays to detect H. contortus infections, and/or (c) as tools to recover desired H. contortus proteins from a mixture of proteins and other contaminants.

In one aspect of the invention, a diagnostic kit for the detection of H. contortus infection is provided comprising a polypeptide sequence of the invention as described in Table 1 and FIGS. 1 to 12.

The present invention further relates to methods for identification of H. contortus infection in an animal by detecting the expression of genes encoding polypeptides of the present invention. The methods comprise analyzing tissue or body fluid from the animal for H. contortus-specific antibodies, nucleic acids, or proteins. Analysis of nucleic acid specific to H. contortus is assayed by PCR or hybridization techniques using nucleic acid sequences of the present invention as either hybridization probes or primers.

Where diagnosis of a disease state related to infection with Haemonchus has already been made, the present invention is useful for monitoring progression or regression of the disease state. For this purpose, a biological sample may be taken.

By “biological sample” is intended any biological sample obtained from an animal, cell line, tissue culture, or other source which contains H. contortus polypeptide, mRNA, or DNA. Biological samples include body fluids (such as saliva, blood, plasma, urine, mucus, synovial fluid, etc.) tissues (such as muscle, skin, and cartilage) and any other biological source suspected of containing Hemonchus contortus polypeptides or nucleic acids.

The present invention is useful for detecting infection related to Haemonchus infections in animals. Preferred animals include sheep, goats, cattle and wild ruminants.

The present invention also provides immunogenic compositions comprising one or more sequences of the present invention as described in Table 1 and FIGS. 1 to 12. Heterogeneity in the composition of an immunogenic composition may be provided by combining one or more H. contortus polypeptides of the present invention. Heterogeneity in the composition multi-component immunogenic composition of this type are desirable because they are likely to be more effective in eliciting protective immune responses against multiple species and strains of the Haemonchus genus than single one.

In one aspect of the invention, an immunogenic composition is provided for the prophylaxis or treatment of H. contortus infection comprising a sequence of the invention and a pharmaceutically acceptable carrier.

In another aspect of the invention an immunogenic composition further comprises an adjuvant.

In yet another aspect of the invention, the immunogenic composition comprises in addition at least one immunogenic sequence derived from another Trichostrongylidae other than Haemonchus. Other Trichostrongylidae include, for example, Trichostrongylus, Ostertagia, Nematodirus, Cooperia, and Hyostrongylus.

The immunogenic composition of the present invention can also include DNA vaccines. Such DNA vaccines contain a nucleotide sequence encoding one or more H. contortus polypeptides of the present invention oriented in a manner that allows for expression of the subject polypeptide.

The administration of the immunogenic composition may be for either a “prophylactic”; or “therapeutic” purpose. When provided prophylactically, the immunogenic composition is provided in advance of any symptoms of H. contortus infection. The prophylactic administration of the immunogenic composition serves to prevent or attenuate any subsequent infection.

In one embodiment of the invention provided is the use of a polypeptide of the invention encoding a H. contortus protein for the manufacture of an immunogenic composition for prophylaxis or treatment of H. contortus infection.

In another embodiment the immunogenic composition comprises a pharmaceutically acceptable carrier.

In yet another embodiment of the invention the immunogenic composition further comprises an adjuvant.

Immunogenic compositions of the present invention can be formulated in an excipient that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m or o-cresol, formalin and benzyl alcohol. Standard formulations will either be liquid injectables or solids which can be taken up in a suitable liquid as a suspension or solution for injection. Thus, in a non-liquid formulation, the excipient may comprise dextrose, human serum albumin, preservatives, etc., to which sterile water or saline could be added prior to administration.

The immunogenic composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel®, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the hall-life of a therapeutic composition in the treated animal. Suitable carriers include, but are not limited to, polymeric controlled release formulations, biodegradable implants, liposomes, bacteria, other viruses, oils, esters, and glycols.

In order to protect an animal from H. contortus infection, an immunogenic composition of the present invention is administered to the animal in an effective manner such that the composition is capable of protecting that animal from infection. For example, it is able to elicit (i.e., stimulate) an immune response, preferably including both a humoral and cellular response, that is sufficient to protect the animal from infection.

Similarly, an antibody of the present invention, when administered to an animal in an effective manner, is administered in an amount so as to be present in the animal at a titer that is sufficient to protect the animal from infection, at least temporarily. Nucleic acid sequences of the present invention, preferably oligonucleotides, can also be administered in an effective manner, thereby reducing expression of H. contortus proteins in order to interfere with parasite development.

Immunogenic compositions of the present invention can be administered to animals prior to parasite infection in order to prevent infection and/or can be administered to animals after parasite infection in order to treat disease caused by the parasite.

Acceptable protocols to administer immunogenic compositions in an effective manner include individual dose size, number of doses, frequency of dose administration, and mode of administration. Determination of such protocols can be accomplished by those skilled in the art. A suitable single dose is a dose that is capable of protecting an animal from H. contortus infection when administered one or more times over a suitable time period.

According to one embodiment, polynucleotide sequences of the present invention can also be administered to an animal in a fashion to enable expression of the nucleic acid sequence into a protective protein in the animal to be protected from H. contortus infection. Nucleic acid sequences can be delivered in a variety of methods including, but not limited to, direct injection (e.g., as “naked” DNA or RNA molecules, packaged as a recombinant virus particle vaccine, and packaged as a recombinant cell vaccine.

A recombinant virus particle vaccine of the present invention includes a recombinant molecule of the present invention that is packaged in a viral coat and that can be expressed in an animal after administration. Preferably, the recombinant molecule is packaging-deficient. A number of recombinant virus particles can be used, including, but not limited to, those based on alphaviruses, pox viruses, adenoviruses, herpes viruses, and retroviruses. Preferred recombinant particle viruses are those based on alphaviruses, with those based on Sindbis virus, Semliki virus, and Ross River virus being more preferred.

When administered to an animal, the recombinant virus particle vaccine infects cells within the immunized animal and directs the production of a H. contortus protein or RNA that is capable of protecting the animal from infection. A preferred single dose of a recombinant virus/particle vaccine of the present invention is from about 1×104 to about 1×105 virus plaque forming units (pfu) per kilogram body weight of the animal. Administration protocols are similar to those described herein for protein-based vaccines.

A composition is said to be “pharmacologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient animal.

While in all instances the vaccine of the present invention is administered as a pharmacologically acceptable compound, one skilled in the art would recognize that the composition of a pharmacologically acceptable compound varies with the animal to which it is administered.

As would be understood by one of ordinary skill in the art, when the vaccine of the present invention is provided to an animal, it may be in a composition which may contain salts, buffers, adjuvants, or other substances which are desirable for improving the efficacy of the composition. The immunogenic compositions of the present invention can be administered parenterally by injection, rapid infusion, nasopharyngeal absorption (intranasopharangeally), dermoabsorption, or orally. The compositions may alternatively be administered intramuscularly, or intravenously.

Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

Many different techniques exist for the timing of the immunizations when a multiple administration regimen is utilized. It is possible to use the compositions of the invention more than once to increase the levels and diversities of expression of the immunoglobulin repertoire expressed by the immunized animal.

According to the present invention, an “effective amount” of a therapeutic composition is one which is sufficient to achieve a desired biological effect. Generally, the dosage needed to provide an effective amount of the composition will vary depending upon such factors as the animal's or human's age, condition, sex, and extent of disease, if any, and other variables which can be adjusted by one of ordinary skill in the art.

The immunogenic compositions of the invention can be administered by either single or multiple dosages of an effective amount.

The effective dosage may vary depending on the mode of administration. If administered intramuscularly, subcutaneously, intradermally or intravenously, effective dosages may depend on the age of the animal. Typically, for an antigen vaccine, dosages are in the range of from about 2 μg to about 1000 μg protein per injection per animal and more preferably 20 μg to about 200 μg protein per injection per animal. More typically, the dose is at least about 50-100 μg protein per injection per animal. Typically, a primary immunization is given followed by one or more booster immunizations given 2-8 weeks apart. Other modes of administration are contemplated by the present invention and include intranasal, intraperitoneal, intrathecal, rectal, infusion and intrapulmonary administration. Administration may also be by injection, nasal drip, aerosol, infusion through the skin or membrane surfaces or ingestion.

A particularly useful form of an immunogenic composition is a vaccine, in particular, a recombinant vaccine comprising a vaccine vector, such as but not limited to a virus vector (e.g. a vaccinia virus vector) or bacterial cell capable of expressing the above mentioned polynucleotides or the vaccine may comprise the polypeptides produced by a vaccine vector.

The present invention clearly extends to recombinant vaccine compositions in which the above mentioned molecules at least is contained within killed vaccine vectors prepared, for example, by heat, formalin or other chemical treatment, electric shock or high or low pressure forces. According to this embodiment, the above mentioned molecules of the vaccine is generally synthesized in a live vaccine vector which is killed prior to administration to an animal. Alternatively a live vector or nucleic acid molecule may be administered.

Furthermore, the vaccine vector expressing the above mentioned molecules may be non-pathogenic or attenuated. Within the scope of this embodiment are non-pathogenic or attenuated viruses and bacteria which express the above mentioned molecules and non-pathogenic or attenuated viruses which express the above mentioned molecules and which are contained within a non-pathogenic or attenuated host cell.

Attenuated or non-pathogenic host cells include those cells which are not harmful to an animal to which the subject vaccine is administered. As will be known to those skilled in the art, “live vaccines” can comprise an attenuated virus vector expressing the above mentioned molecules or a host cell comprising same, which is capable of replicating in an animal to which it is administered, albeit producing no adverse side-effects therein. Such vaccine vectors may colonize the gut or other organ of the vaccinated animal. Such live vaccine vectors are efficacious by virtue of their ability to continually express the above mentioned molecules in the host animal for a time and at a level sufficient to confer protective immunity against a pathogen which expresses an immunogenic equivalent of the said above mentioned molecules. The present invention clearly encompasses the use of such attenuated or non-pathogenic vectors and live vaccine preparations.

The vaccine vector may be a virus, bacterial cell or a eukaryotic cell such as an avian, porcine or other mammalian cell or a yeast cell or a cell line such as COS, VERO, HeLa, mouse C127, Chinese hamster ovary (CHO), WI-38, baby hamster kidney (BHK) or MDCK cell lines. Suitable prokaryotic cells include Mycobacterium spp., Corynebacterium spp., Salmonella spp., E. coli, Bacillus spp. and Pseudomonas spp, amongst others. Bacterial strains which are suitable for the present purpose are well-known in the relevant art (Ausubel et al, 1987; Sambrook et al, 1989). Suitable viral vectors include but are not limited to vaccinia virus and adenovirus.

Such cells and cell lines are capable of expression of a genetic sequence encoding the above mentioned molecules of the present invention in a manner effective to induce a protective effect in the animal. For example, a non-pathogenic bacterium could be prepared containing a recombinant sequence capable of encoding the above mentioned molecules. The recombinant sequence would be in the form of an expression vector under the control of a constitutive or inducible promoter. The bacterium would then be permitted to colonize suitable locations in an animal's gut and would be permitted to grow and produce the recombinant form of the above mentioned molecules in amount sufficient to induce a protective response against a nematode.

The vaccine can be a DNA vaccine comprising a DNA molecule encoding the above mentioned proteins of the present invention and which is injected into muscular tissue or other suitable tissue in an animal under conditions sufficient to permit transient expression of said DNA to produce an amount of the above mentioned molecules effective to induce a protective response.

In the production of a recombinant vaccine, except for a DNA vaccine described herein, it is necessary, therefore, to express the above mentioned molecules in a suitable vector system. For the present purpose, the above mentioned molecules can be expressed by:
(i) placing an isolated nucleic acid molecule of the invention in an expressible format,
(ii) introducing the isolated nucleic acid molecule of (i) in an expressible format into a suitable vaccine vector; and
(iii) incubating or growing the vaccine vector for a time and under conditions sufficient for expression of the immunogenic component encoded by said nucleic acid molecule to occur.

As used herein, a “nucleic acid molecule in an expressible format” is a protein-encoding region of a nucleic acid molecule placed in operable connection with a promoter or other regulatory sequence capable of regulating expression in the vaccine vector system.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operatively associated, and which encodes the above mentioned molecules. Preferred promoters can contain additional copies of one or more specific regulatory elements, to further enhance expression and/or to alter the spatial expression and/or temporal expression of the said nucleic acid molecule.

Placing a nucleic acid molecule under the regulatory control of i.e., “in operative association with” or “operably linked to” a promoter sequence means positioning the molecule such that expression is controlled by the promoter sequence. Promoters are generally, but not necessarily, positioned 5′ (upstream) to the genes that they control.

For DNA vaccines, a preferred amount is from about 0.1 μg/mL to about 5 mg/mL in a volume of about 0.05 to about 5 mL. The DNA can be present in “naked” form or it can be administered together with an agent facilitating cellular uptake (e.g., in liposomes or cationic lipids). The important feature is to obtain sufficient expression of the nucleotide sequence encoding the immunogen in the cells of the animal after injection to induce a protective immune response. Dosage regime can be adjusted to provide the optimum therapeutic response.

Although the present invention is exemplified in relation to the isolation and the use of the above sequences and proteins derived from H. contortus, the present invention extends to the above mentioned sequences and proteins from all helminths including trematodes (e.g. of the genera Fasciola and Schistosoma), cestodes (tapeworms), nematodes (roundworms) and acanthocephala (thornyheaded worms) which cause severe diseases in humans and animals. The teaching of the present specification enables the isolation of analogous molecules for use in combating a range of helminth infection.

One important target group of worms in accordance with the present invention is the nematode group which can cause severe diseases in mammals and fowl, for example in sheep, pigs, goats, cattle, horses, donkeys, dogs, cats, guinea pigs, cage-birds. Typical representatives of such nematodes are: Haemonchus, Trichostrongylus, Ostertagia, Nematodirus, Cooperia, Ascaris, Bunostomum, Oesphagostomum, Charbertia, Trichuris, Strongylus, Trichonema, Dictyocaulus, Capillaria, Heterakis, Toxocara, Ascaridia, Oxyuris, Ancylostoma, Uncinaria, Toxascaris and Parascaris.

Certain species of the genera Nematodirus, Cooperia, Trichostrongylus and Oesphagostomum attack the intestinal tract of the host animal, whereas other species of the genera Haemonchus, Trichostrongylus and Ostertagia parasitize the stomach and species of the genera Dictyocaulus and Muellerius parasitize the lung tissue. Parasites of the families Filariidae and Setariidea are found in internal cell tissue and internal organs, e.g. in the heart, blood vessels, lymph vessels and in subcutaneous tissue. In this connection, particular mention is to be made of the dog heartworm, Dirofilaria immitis.

Important nematodes parasitic in dogs and cats embraceD. immitis; D. repens; Toxocara cati; T. canis; Toxascaris leonina; Ancylostoma tubaeforme; A. caninum; A. braziliense, Uncinara stenocephala; and Trichuris vulpis.

Another aspect of the present invention is the successful control of pathogenic nematodes in humans such as those which occur in the alimentary tract. Typical representatives of this type belong to the genera Ancylostoma, Necator, Ascaris, Strongyloides, Trichinella, Capillaria, Trichuris and Enterobius. Other important parasitic nematodes of the genera Wuchereria, Brugia, Onchocerca and Loa of the family Filariidae and the genus Dracunculus of the family Dracunculidae, which occur in the blood, in tissue and various organs, are also encompassed by the present invention.

The following examples are provided for the purposes of illustration and are not intended to limit the scope of the invention.

### EXAMPLES

### Example 1

### Preparation of cDNA and Subtracted cDNA Libraries from H. contortus

Unless otherwise stated, molecular biology methods were as described by Sambrook et al. (1989, supra) or Sambrook and Russell (A Molecular Cloning: A Laboratory Manual. Third Edition. Cold Spring Harbor Laboratories, Cold Spring Harbor, N.Y., 2001).

Materials from several parasite stages used for the generation of cDNA and subtracted cDNA libraries are obtained as follows. A benzimidazole-resistant line of H. contortus is maintained by serial passage in 3-6 month-old, helminth-free Merino weaner sheep. Faecal cultures from weaners with a patent infection are harvested to recover infective third-stage larvae (L3) after 6-7 days. L3 are exsheathed by exposure to CO2 (xL3) and separated from cuticular casts by migration through two 20 μm nylon screens. xL3 are axenised in an antibiotic solution, then suspended in medium containing sheep serum and cultured under in vitro conditions to produce early fourth-stage larvae (eL4, Nikolaou et al, 2002). To provide the blood feeding stage (eL4bf), blood products are included in the medium during the culture period. Mature adult H. contortus are recovered after 35-175 days from the abomasa of monospecifically infected sheep. For the isolation of gut-specific molecules, the intestines (“gut”) are manually dissected from adult female H. contortus (35-56 days infections). Subtracted cDNA libraries are generated using a subtractive suppressive hybridisation method, utilising Clonetech's PCR-select cDNA subtraction kit, which allows for the selection of differentially regulated transcripts between two mRNA populations. In this procedure, the two mRNA populations are converted to cDNA. The cDNA that contains the genes of interest is termed the “tester” and the cDNA that contains the genes to be removed is termed the “driver”. Both cDNA populations are hybridised and during the PCR step hybrid molecules are removed, with the remaining unhybridised cDNA representing genes that are expressed in the tester population.

cDNA libraries are constructed from mRNA from specific parasitic material using the Lambda ZAP II (AZAPII) vector (Stratagene). cDNA fragments between 0.5 and 2 kb in length are ligated into the EcoRI site of the vectors under conditions specified by the manufacturer (Stratagene). The number of primary clones in the libraries generated is approximately 10⁶ plaque forming units/mL. The libraries are amplified prior to use. A custom cDNA library is constructed in Lambda TriplEx (λTriplEx) vector by Clontech laboratories Inc. U.S.A. using oligo (dT) and random priming. cDNA fragments between 0.5 and 3.6 kb in length are ligated into the Eco RI site of the vector. The number of primary clones in the library is 3.3×10⁶ plaque forming units/mL. The library is amplified to a titre of >10⁹ pfu/mL before use.

### Example 2

### mRNA Expression Profiling Using Quantitative PCR

Adult female H. contortus, obtained as stated above, are manually dissected to obtain the intestine (“gut”), reproductive (“ovary”) and muscle (“body”) tissues, which are immediately placed in aliquots of TriPure™ Isolation Reagent (Roche Molecular Biochemicals). Total RNA is extracted from these TriPure™ samples according to the manufacturer's instructions, including the optional step to remove the extracellular material. mRNA is then extracted using the Poly(A)Pure™ kit (Ambion), as per manufacturer's instructions. The mRNA is resuspended in DEPC water with RNasin® (Promega) to prevent degradation.

cDNA is synthesised as described in Nikolaou et al., 2004, and diluted from 1:10 up to 1:50, depending on the concentration of the sample, for real-time PCR. Real time PCR is carried out as described in Nikolaou et al., 2004, looking only at total expression (not at isoforms), and using 2 μl of the FastStart™ DNA Master SYBR Green I mix. Cycling is carried out in the same manner, with annealing temperatures (60-68C) and extension times being molecule dependent.

### Example 3

### cDNA Library Screening or PCR Amplification of Full Length cDNA Clones

cDNA library screening is undertaken to isolate full length cDNA clones for the corresponding target molecules. In general, H. contortus cDNA libraries are probed using ³²P labelled cDNA fragments. The probes are labelled using α-³²P dCTP by random priming using Klenow under conditions specified by the manufacturers (Promega). Generally 1×10⁵ plaques are hybridised to the probe in the hybridisation buffer (7% (w/v) SDS, 1 mM EDTA, and 0.25 M sodium phosphate pH6.5) overnight at 65° C. Filters are washed in copious amounts of wash solution (2×SSC, 0.1% SDS (w/v)) at 65° C. This is generally done in 3 washes or until no radioactivity could be detected in the washings. Washed filters are exposed to x-ray film, and positive clones are excised according to the method recommended for the particularly cDNA library. Excised cDNA clones are sequenced using Dye Terminator DNA sequencing, using fluorescent dyes. This is done using a Perkin Elmer/ABI cycle sequencing kit as per the manufacturer's instructions. Sequencing primers used in the sequencing reactions are vector specific.

Primers (typically 25mers) are designed to amplify the ORF, minus any signal sequence or transmembrane domain, from gut cDNA using standard PCR techniques. The PCR product of the correct size is cut from a 1% agarose gel (TAE buffer), and the DNA extracted using the Promega Wizard® SV Gel and PCR Clean-up System, according to the manufacturer's instructions. The PCR fragments are poly-A-tailed and ligated into pGEM T Easy, as per the manufacturer's instructions. This clone is then used as template for generation of the attB PCR fragment for Gateway cloning.

### Example 4

### Preparation of Recombinant Proteins

Expression and Purification of Recombinant Proteins in E. coli

Recombinant proteins are produced in E. coli BL21 (DE3) pLysS cells (Stratagene) (subsequently referred to as BL21 cells) using the Gateway vector pDEST17. cDNA fragments are cloned into the pDEST17 Destination expression vector using Gateway™ Technology (Invitrogen) as per the manufacturer's instructions. Target specific primers including attB recognition sequences are used to generate a PCR product for cloning into the pDONR207 Gateway vector. The resulting entry clone is recombined with pDEST-17 Destination vector to give rise to an expression clone for E. colt 10-50 ng of purified pDEST17-target plasmid is transformed into 25 μL of chemically competent BL21 E. coli cells. The reaction is incubated on ice for 20 min, heat shocked at 42° C. for 45-90 seconds and immediately placed back on ice. The reaction is incubated for a further 45 min at 37° C. after the addition of 300 μL LB broth. Cells are recovered by centrifugation and the supernatant removed. The cells are resuspended in 200 μL of LB broth prior to plating on LB agar supplemented with ampicillin (100 μg/mL) and chloramphenicol (30 μg/mL) to select for positive transformants. Plates are incubated overnight at 37° C. to allow for bacterial growth.

Large-scale production of recombinant protein is performed in shaker flasks. 500 mL overnight cultures are grown from single colonies of pDEST17-target plasmid in BL21 cells in LB broth supplemented with ampicillin (100 μg/mL) and chloramphenicol (30 μg/mL). Fresh 500 mL broths are inoculated with 50 mL of the overnight cultures and grown at 37° C. with shaking until the optical density at 600 nm (OD₆₀₀) reached 0.6-0.8. IPTG (iso-propyl β-D-thiogalactopyranoside) is added to a final concentration of 1 μM to induce expression of the recombinant protein and the cultures grown for a further 3 hrs. The cultures are harvested by centrifugation at 4° C. at 7,000 rpm for 20 min. The cell pellets are resuspended in 60 mL cold binding buffer (500 mM NaCl, 20 mM Tris, pH 8.0, 5 mM imidazole) and the cells disrupted by sonication (8 bursts of 10 sec on ice). The sonicate is centrifuged at 10,000 rpm for 15 min at 4° C. and supernatant (aqueous fraction) removed. The pellet is resuspended in 60 mL binding buffer plus 8 M urea and rotated at 4° C. for 1 hr. The solution is centrifuged at 10,000 rpm for 15 min at 4° C. and the supernatant (urea soluble fraction) harvested and filtered through a 0.8 μm filter. To the filtrate is added a 1 mL bed volume of nickel-NTA beads (QIAGEN). The filtrate and beads are rotated at 4° C. for a minimum of 1 hr. The beads are washed with a minimum of 25 volumes of binding buffer plus 8M urea. The bound recombinant protein is eluted in 100-400 mM imidazole in binding buffer plus 8M urea, and samples of the eluted fractions analysed by SDS-PAGE using a 12.5% (w/v) resolving gel. Fractions, which contained the highest purity of recombinant protein (approximately 95% pure), are pooled and total protein estimated using the Micro BCA Protein Assay Reagent Kit (Pierce). The concentration of the recombinant protein is adjusted for vaccine formulation using binding buffer plus urea and the antigen frozen in aliquots at −70° C. Recombinant proteins are transported on dry ice for use in vaccine trials. Aliquots of the protein are also retained for use in the assessment of antibody responses.

### Expression and Purification of Recombinant Protein in Baculovirus Infected Insect Cells

Recombinant proteins are produced using the recombinant baculovirus/insect cell system. The FastBac system (Invitrogen) is used. Unless otherwise specified, all vectors, cells, reagents and media are from Invitrogen.

### Subcloning, Transposition and Transfection

The pHeskVecB and pHeskVec-Destination vectors are constructed using the vector pGp67/pFastBac provided by Heska Corporation Pty Ltd (this vector contained a signal peptide from the baculovirus acidic glycoprotein gp67). pGp67/pFastBac did not contain a His-tag, therefore, pHeskVecB is engineered to include a 6×His-tag located downstream from the Gp67 signal sequence and upstream of the multiple cloning site. pGp67/pFastBac is digested with EcoRI and RsrII, and gel purified before ligating to linkers containing a 6×His-tag, the linker is generated using the oligonucleotides His-linker Forward (SEQ ID 1,5′-GTCCCACCATCACCATCACCATACCATGGG-3′) and His-linker Reverse (SEQ ID 2,5′-AATTCCCATGGTATGGTGATGGTGATGGTGG-3′), giving rise to pHeskVecB. pHeskVec-Destination is constructed using pHeskvecB and the rfA Gateway cassette available from Invitrogen. The multiple cloning cassette of pHeskVecB is removed by digestion with NcoI and HindIII, and the overhangs are filled to generate blunt ends. The rfA Gateway cassette is cloned into the blunt ends of the NcoI-HincII-pHeskVecB to create the pHeskVec-Destination expression vector.

Target specific primers including attB recognition sequences are used to generate a PCR product for cloning into the pDONR207 Gateway vector. The resulting entry clone is recombined with pHeskVecB Destination to give rise to an expression clone. The clone is transposed into DH10BAc cells according to the manufacturer's instructions and bacmid DNA purified using the Jetstar kit (GENOMED) according to instructions. The bacmid DNAs are transfected into Spodoptera frugiperda (S19) cells using Cellfectin according to the manufacturer's instructions. S19 cells are maintained in Grace's complete medium containing 10% (v/v) Foetal Calf Serum (FCS) (CSL Ltd). Recombinant virus from the transfection is harvested and amplified. Supernatant from the amplification is used to infect a 100 mL spinner culture of High Five cells (BTI-TN-5B1-4), derived from Trichoplusia ni egg cell homogenates to generate a viral stock. Hi5 cells are maintained in Express five SFM (serum free medium). The viral stock is harvested by centrifuging the cell suspension for 5 minutes at 1,500 rpm.

### Scale-Up Production and Purification of Recombinant Protein

The viral stock is used to infect 250 mL Hi5 cell cultures in 1 L shaker flasks supplemented with 1 μg/mL gentamycin (CSL Ltd) and 10% Glutamax-1 (Gibco) at a multiplicity of infection (MOI) of 0.1. Cultures are harvested 48-72 hours post-infection. Cells are pelleted by centrifugation at 2,500 rpm for 10 minutes. The cell pellets are resuspended in 20 mL cold binding buffer (500 mM NaCl, 20 mM Tris, pH 8.0, 5 mM imidazole) for each original 250 ml cell culture. The cells are disrupted using a Parr Cell Disruption Bomb (Parr) according to the manufacturers instructions. The disrupted cells are centrifuged at 10,000 rpm for 15 min at 4° C. and the supernatant (aqueous fraction) removed. The pellet is resuspended in cold binding buffer plus 1% Triton X-100 (same volume as above) by passing the pellet/buffer mix through a 18 gauge needle 2-3 times and rotating at 4° C. for 1 hr. The solution is centrifuged at 10,000 rpm for 15 min at 4° C. and the supernatant (triton soluble fraction) harvested and removed. The remaining insoluble pellet is resuspended in 15-20 ml of cold binding buffer by passing the pellet through an 18 G needle.

Antigen S2-259MF is present within the insoluble pellet. The final protein pool is analyzed by SDS-PAGE. The samples are diluted in 2× sample buffer, heated to 90° C. for 5 min and 2-10 μl samples electrophoresed on SDS-PAGE gels using a 12.5% (w/v) resolving gel. The concentration of the antigen is determined by comparison with standards of known protein content and adjusted for the vaccine formulation using binding buffer. The recombinant protein is stored at −70° C. and transported on dry ice for use in vaccine trials. Aliquots of the protein are retained for use in the assessment of antibody responses.

The predicted and observed molecular mass of proteins expressed in either E. coli or Baculovirus are listed in Table 2.

### Example 5

### Vaccine Trials In Sheep

Vaccination trials are conducted in sheep. The studies utilized Merino lambs (6 months old at the start of the trial) born and raised under helminth-free conditions. Five lambs are allocated to each of the vaccine groups and five to the control group by restricted randomization on a liveweight basis. For the duration of the trial, lambs are housed on wire mesh flooring. The control groups receive phosphate buffered saline (PBS) (0.17M NaCl, 0.003M KCl, 0.01M Na₂HPO₄, 0.002M KH₂PO₄ pH7.4 (HCl)) and the vaccine groups receive the appropriated recombinant protein antigen. The vaccine or PBS is formulated with Aluminium Hydroxide (5 mg/ml) (Sigma) and Quil-A solution (1 mg/ml) (HCl Biosector) by adding 3.5 ml of PBS or recombinant protein antigen to 1.85 ml of Alum (19 mg/ml stock solution) and mixing with a rotary mixer at RT for 25 minutes. 1.65 ml of Quil A solution (4.2 mg/ml) is added to the mixture and gently inverted. For each lamb, a total vaccine volume of 1 mL is given, generally containing a vaccine dose of 100 μg of the recombinant protein antigen. Lambs are injected on days −70, −42 and −14 via the subcutaneous route into the area above the shoulder.

TABLE 2
Molecular Mass of Proteins

| | | Predicted | Observed |
|---|---|---|---|
| | | Molecular | Molecular |
| | Protein Expression | Mass | Mass |
| Clone | Construct | (kDa)^{a} | (kDa)^{b} |
| S7T104D12 | pDEST17-S7T104D12 | 17.99 | 16, 17 |
| S4T58F9 | pDEST17-S4T58F9 | 32.32 | 32 |
| S1-381 | pDEST17-S1-381#2 | 19.06 | 17, 33 |
| G0142 | pDEST17-G0142-1 | 33.9 | 34 |
| S1T1F1 | pDEST17-S1T1F1#9 | 24.57 | 24, 30 |
| S2-259MF | S2-259MF-Clone10.1 | 57.53 | 65 |
| G1083P | pDEST17-G1083P#1 | 29.44 | 28, 60 |
| dd165-2NTC#1 | pDEST17-dd165-2NTC#1 | 15.96 | 15 |
| S4T69C3 | pDEST17-C69C3-5.5 | 50.12 | 51 |
| YAd189 | pDEST17-YAd189#7.2 | 51.53 | 52 |
| YAd219 | pDEST17-YAd219 | 11.42 | 12 |
| S4T55C6 | pDEST17-S4T55C6 | 35.42 | 32, 34, 36, 70 |

^{a}denotes: predicted molecular mass (kDa) of the recombinant protein as calculated from the amino acid sequence of the expression constructs that includes 2.7 kDa - corresponding to the initiation Methionine, His tag, and additional linker residues from pDEST17 vector sequence.
^{b}denotes: observed molecular mass (kDa) of the purified recombinant protein as determined by comparison to molecular mass standards on SDS-PAGE.

### Example 6

### Assessment of Antibody Reponses

Blood samples (10 mL) are collected from the jugular vein of each lamb on days −70 (prebleed, 1^{st} vaccination), −42 (2^{d} vaccination), −14 (3^{rd} vaccination), 0 (challenge, 2 weeks post 3^{rd} vaccination) and +35 (necropsy). The blood is allowed to coagulate and the serum separated by centrifugation and stored at −20° C. Antibody responses are quantitated by ELISA. 96-well plates are coated with 50 μL/well purified recombinant protein (1 μg/mL) overnight at 4° C. The plates are washed in PBST (PBS containing 0.05% (v/v) Tween 20) then blocked with 5% (w/v) skim milk powder in PBST (PBSTB) (150 μL/well) at 37° C. for a minimum of 30 minutes. Test serum samples are diluted in PBSTB, added to the plates at 100 μL/well and incubated for 1 hour at 37° C. on a rocking table, followed by washing in PBST. Secondary antibody is horse radish peroxidase conjugated donkey anti-sheep IgG (Sigma) used at a dilution of 1/1000 in PBSTB, added at 50 μL/well, and incubated for 1 hour at 37° C. Following further washing with PBST, colour development is achieved with the addition of 100 μL/well of substrate (TMB single solution (ZYMED), TMB: 3,3′,5,5′-tetramethyl-benzidine dihydrochloride) for 20 min. Colour development is stopped by adding 100 μL/well of Stopping solution (0.5M H₂SO₄). Plates are read on a Molecular Devices Vmax kinetic plate reader at a wavelength of 450 nm. Samples are assayed in triplicate and over 2 separate assays. Antibody titres are calculated on the linear portion of the titration curve where the optical density is equal to 1.0. Vaccination with the recombinant protein antigen on days −70, −42 and −14 stimulated a statistically significant higher antibody titre in the day 0 bleed of individual animals relative to the day −70 pre-bleed.

### LEGEND TO FIGURES

FIG. 1—Clone S7T104D12

FIG. 1-1—SEQ ID No.5

mRNA is isolated from eL4 (tester) and xL3 (driver) parasitic preparations. The mRNA preparations are subjected to the subtractive suppressive hybridisation method. Unhybridised cDNA molecules are cloned into pGEMT-Easy (Promega). This is termed the S7 subtracted cDNA library (eL4 minus xL3). The S7 subtracted library is propagated using JM109 Escherichia coli cells. Individual colonies are selected, grown in LB-broth under the selection of ampicillin, and plasmid DNA extracted using plasmid DNA prep columns (Qiagen). The plasmid DNA is checked for purity and the presence of a cDNA insert using a spectrophotometer and restriction endonuclease digestion, respectively. The vector sequencing primer sites are used to sequence the pGEMT-easy subtracted library clone. The flanking vector primers 17 (SEQ ID NO.3, GTAATACGACTCACTATAG) and SP6 (SEQ ID NO.4, ATTTAGGTGACACTATAG) are used. Clone S7T104D12 is found to contain: a 573 base pairs (bp) cDNA insert, including a stop codon (shown in bold) but no start codon, with the following nucleotide sequence of SEQ ID NO. 5 (5′ to 3′).

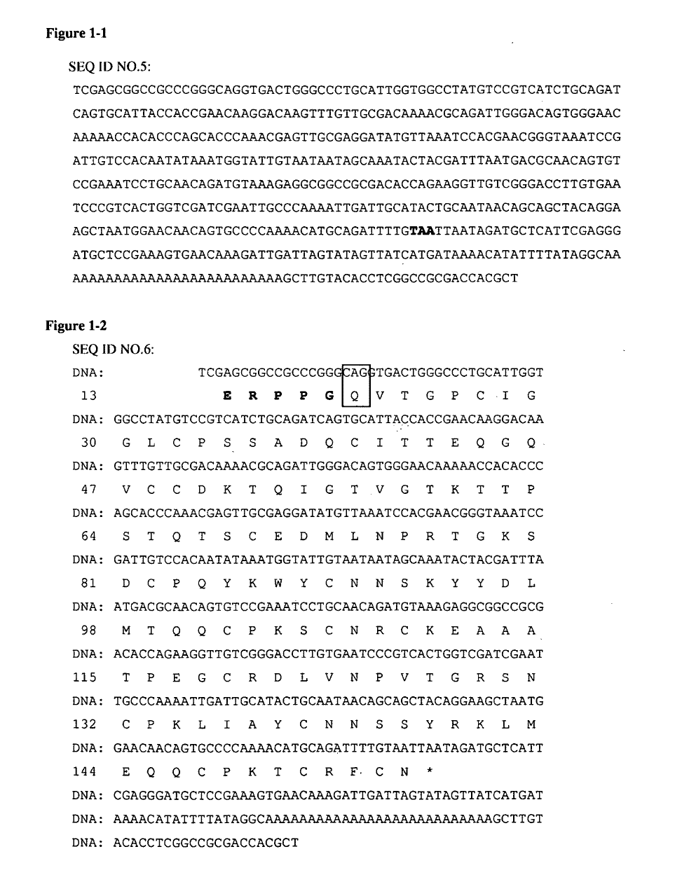

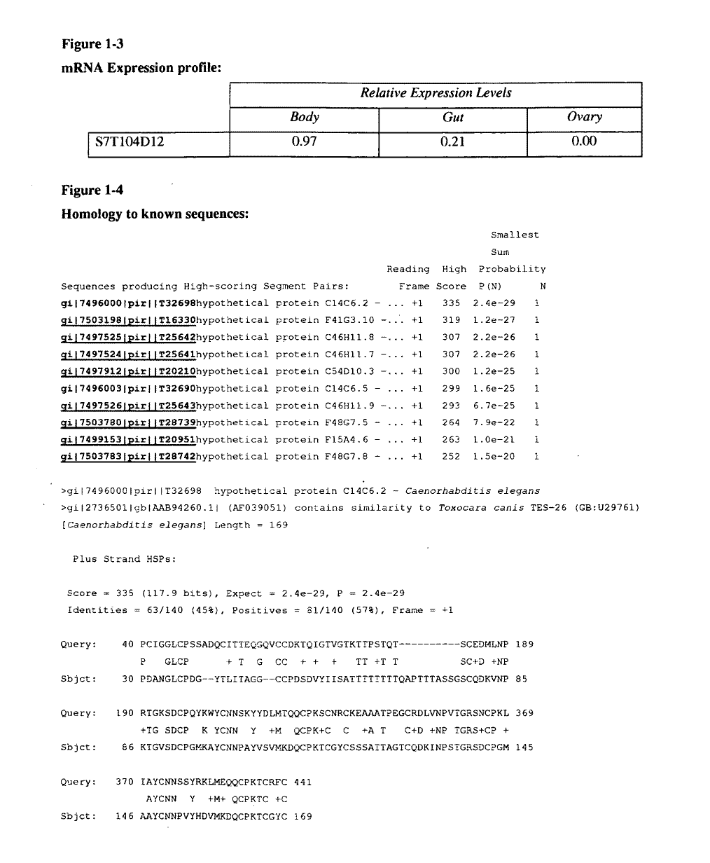

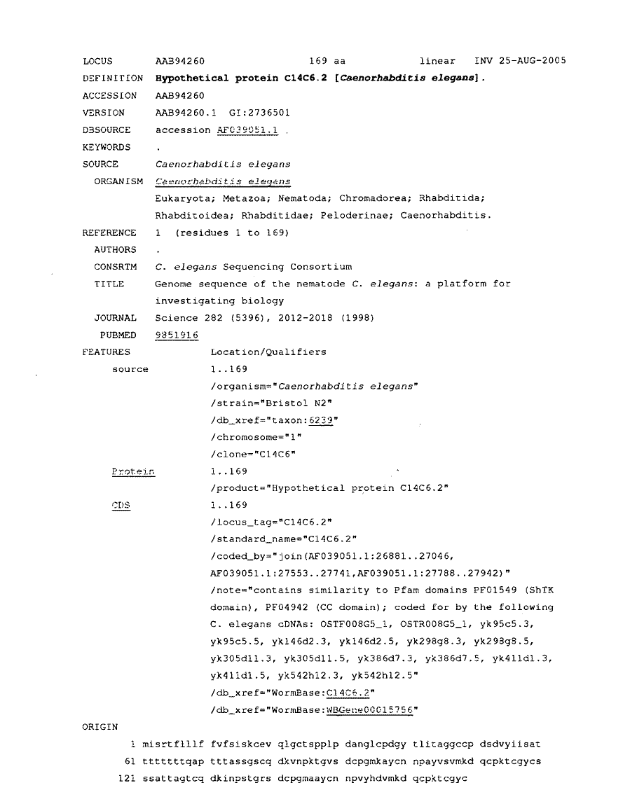

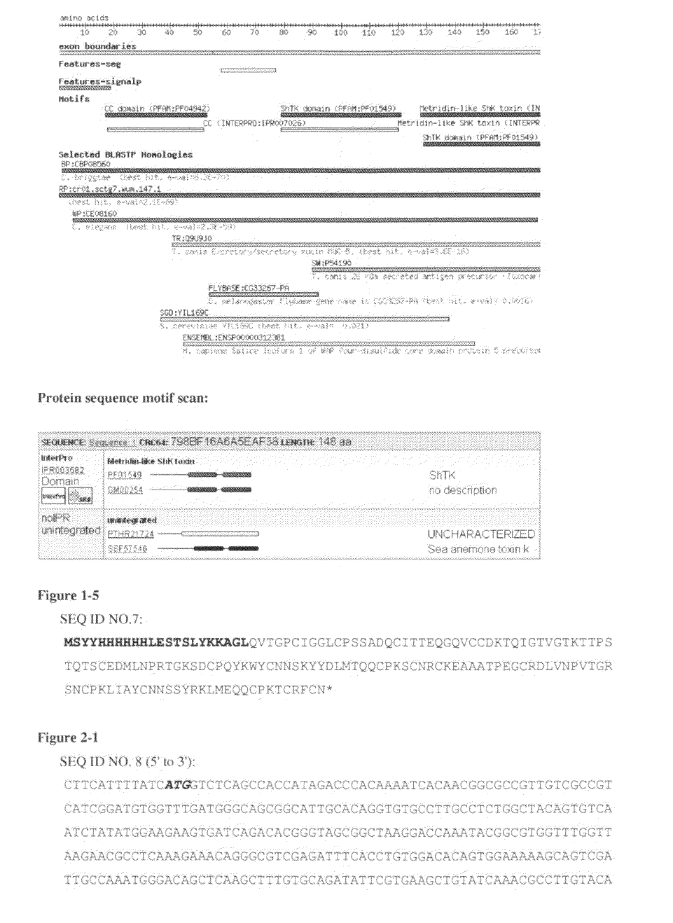

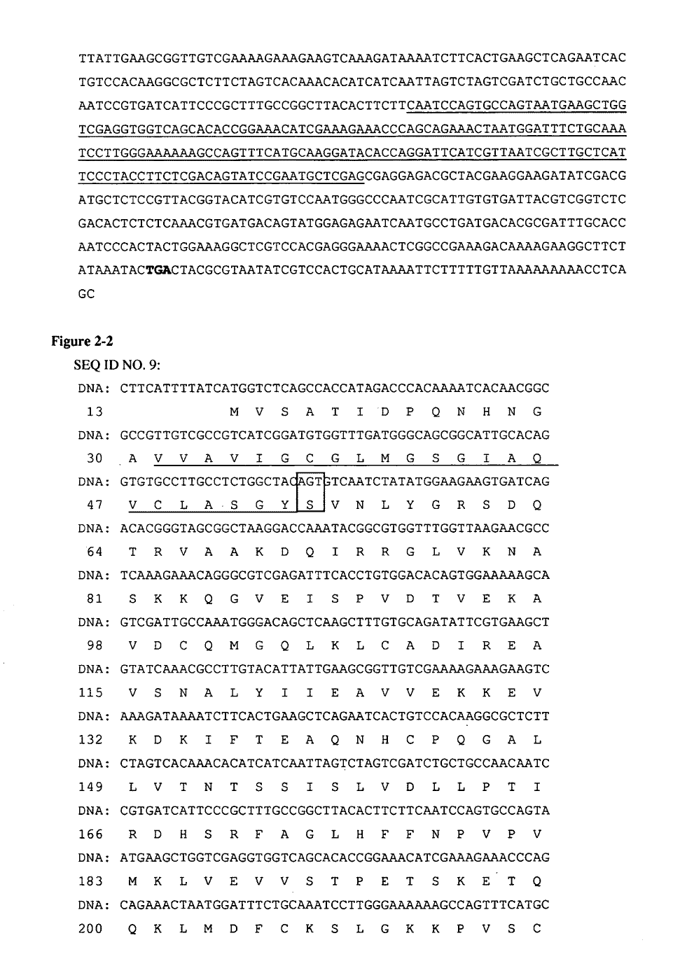

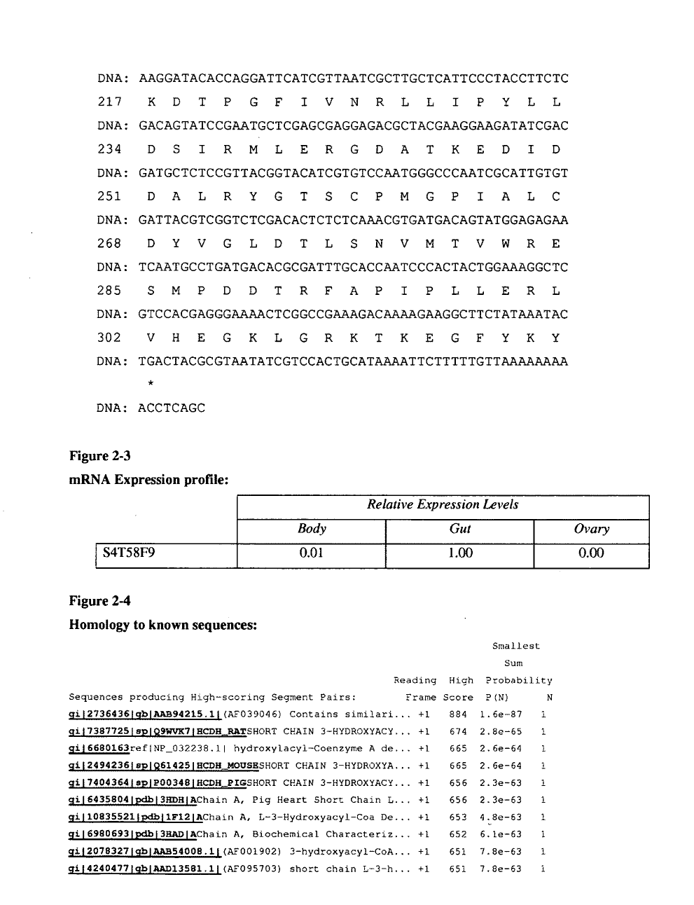

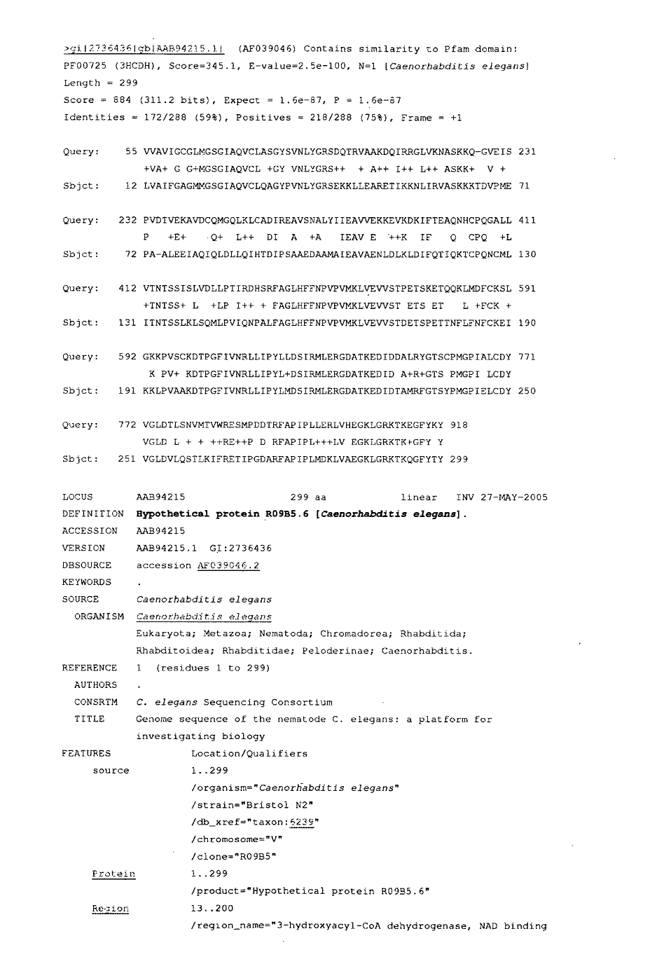

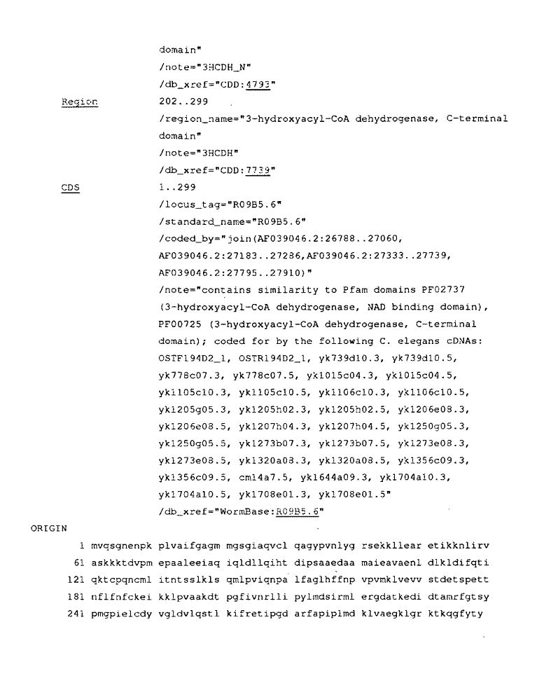

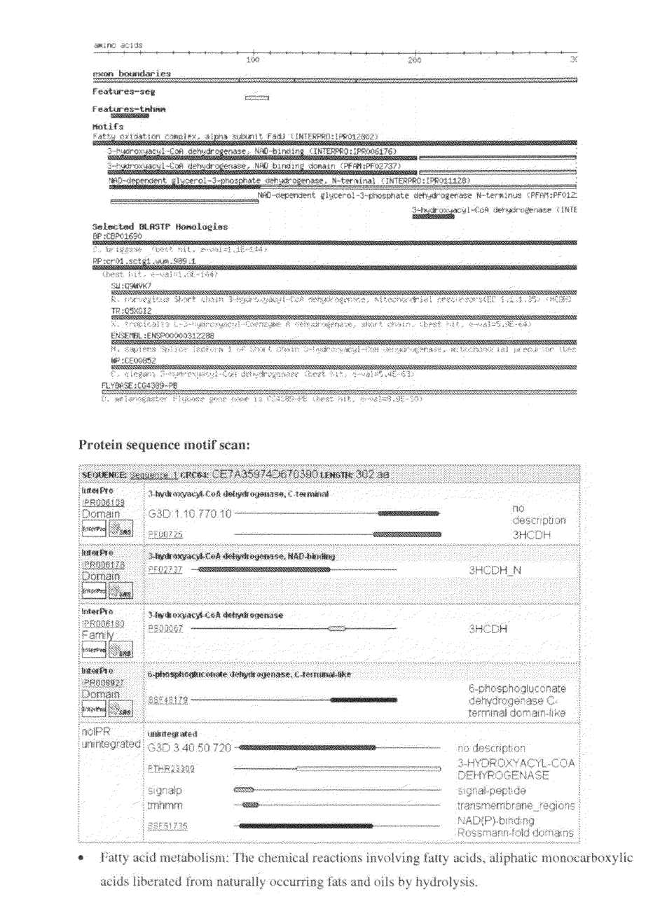

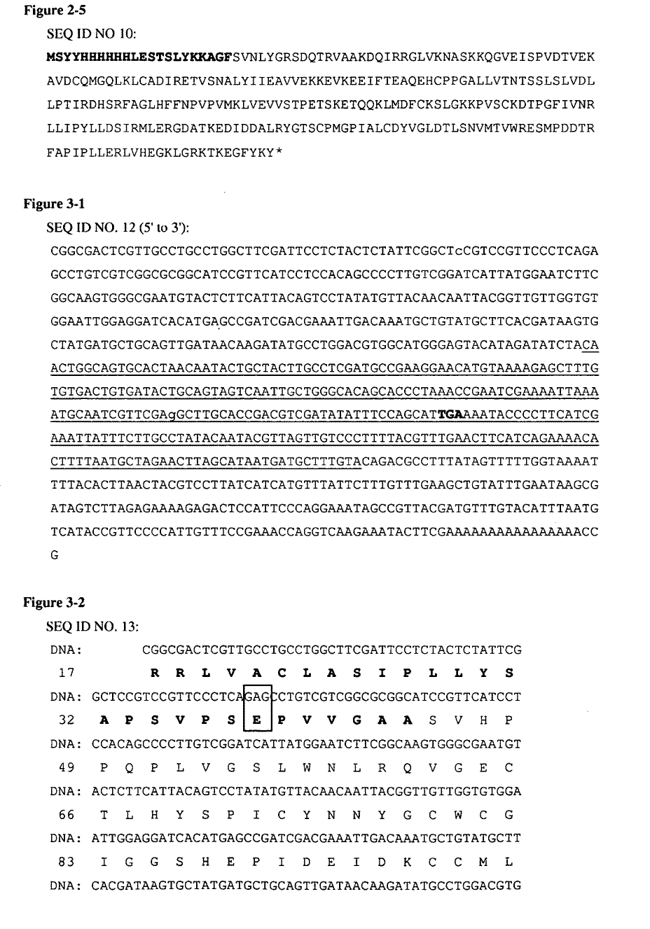

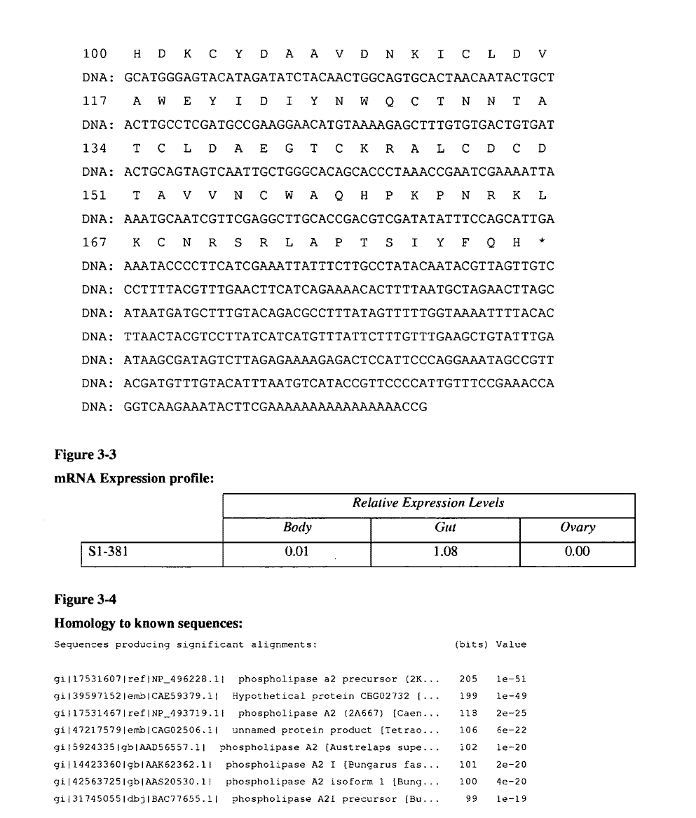

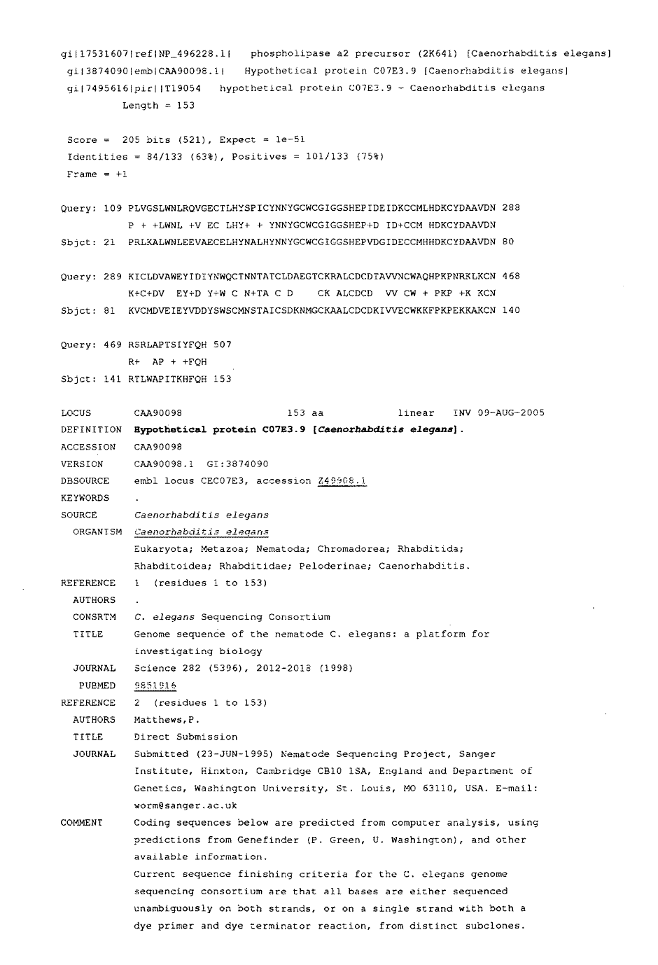

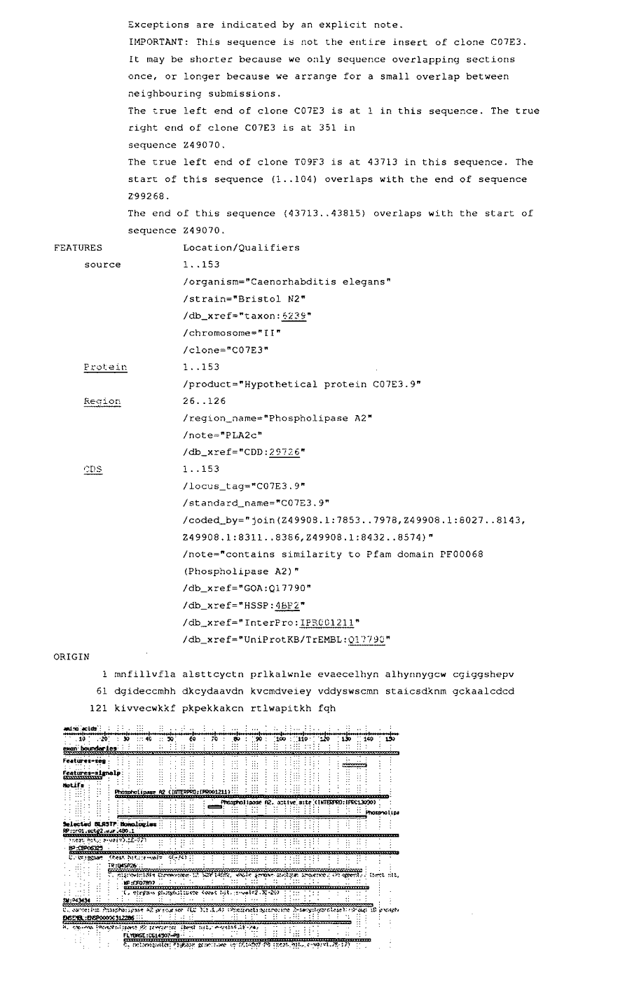

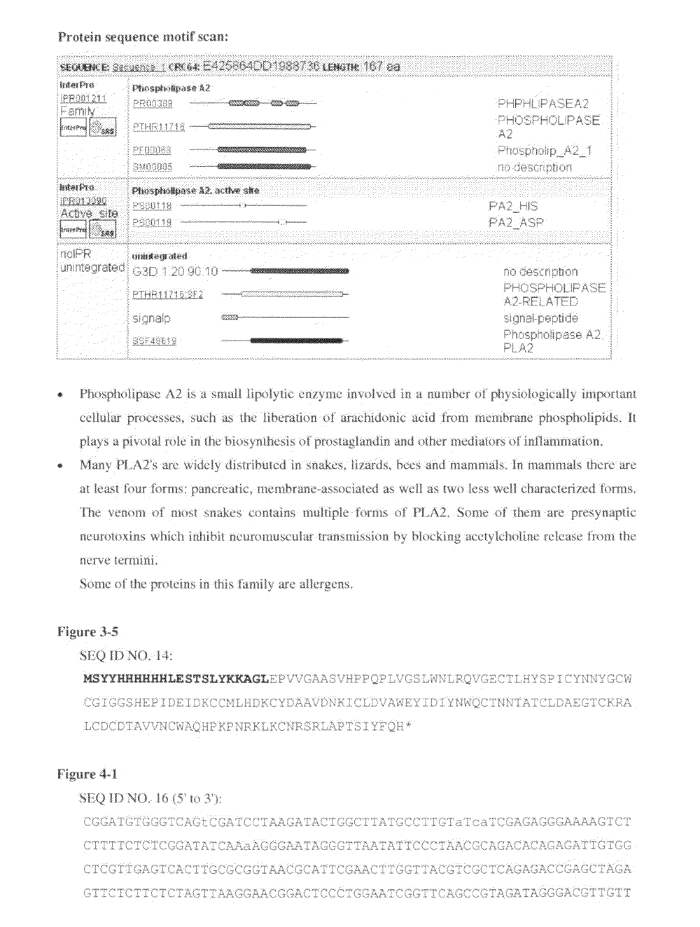

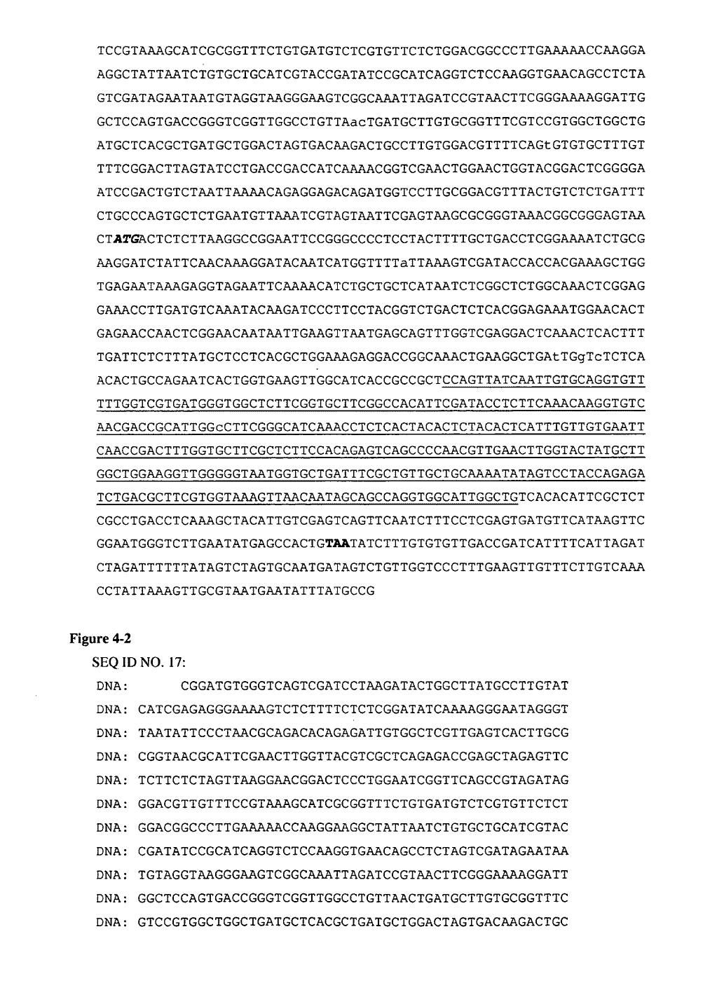

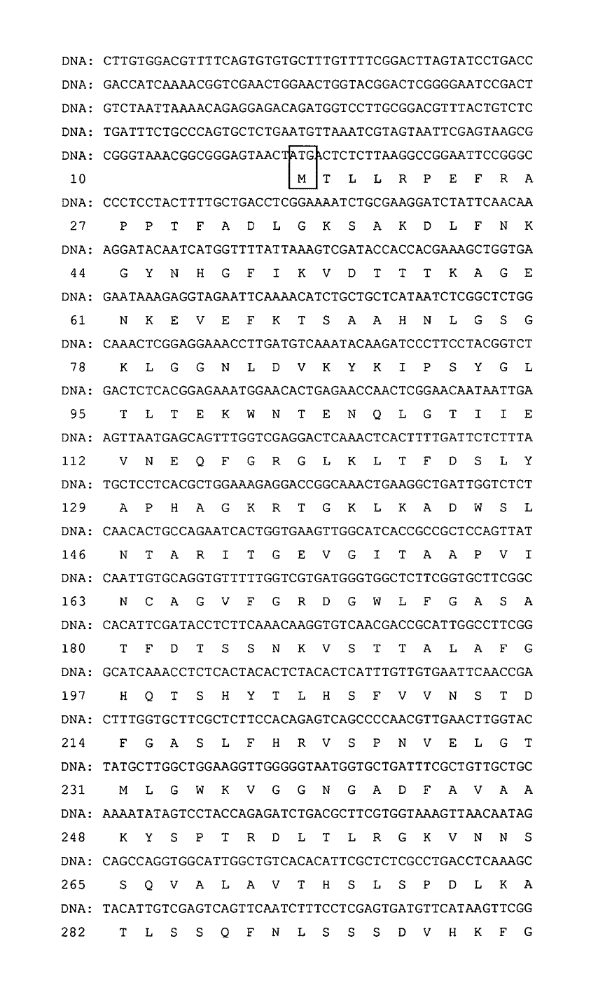

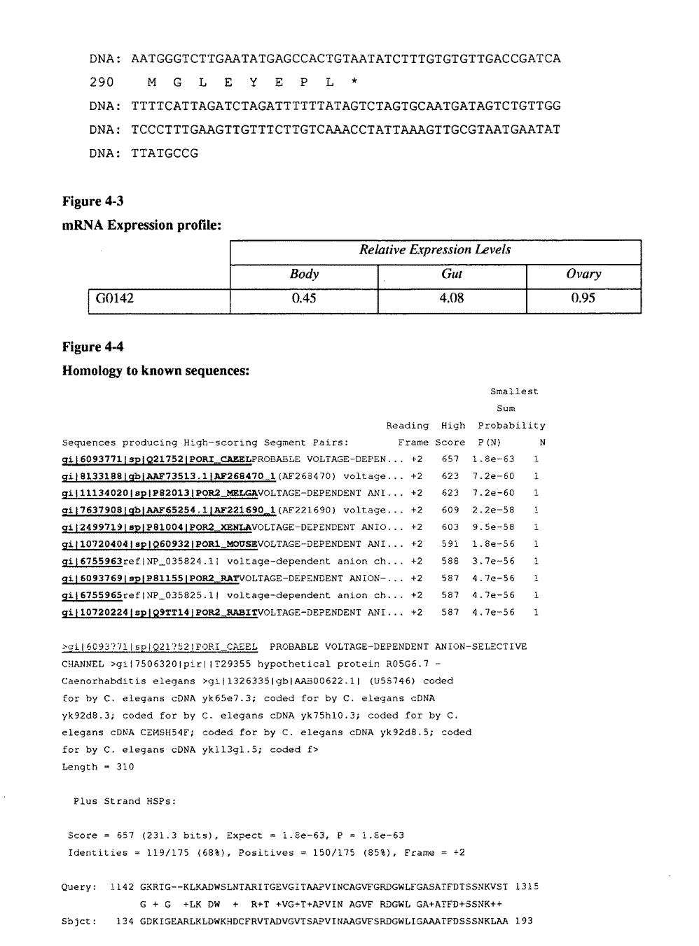

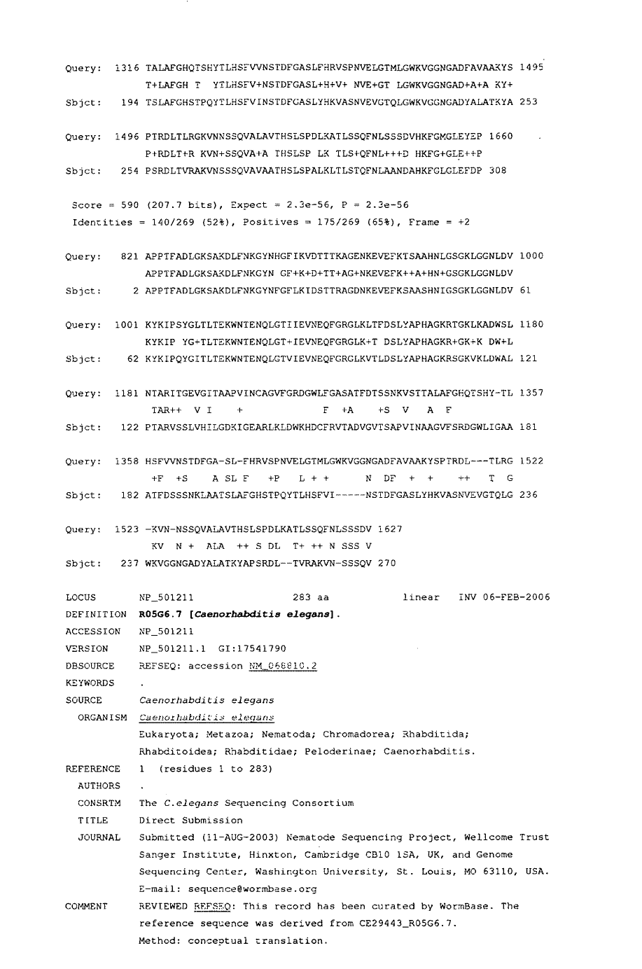

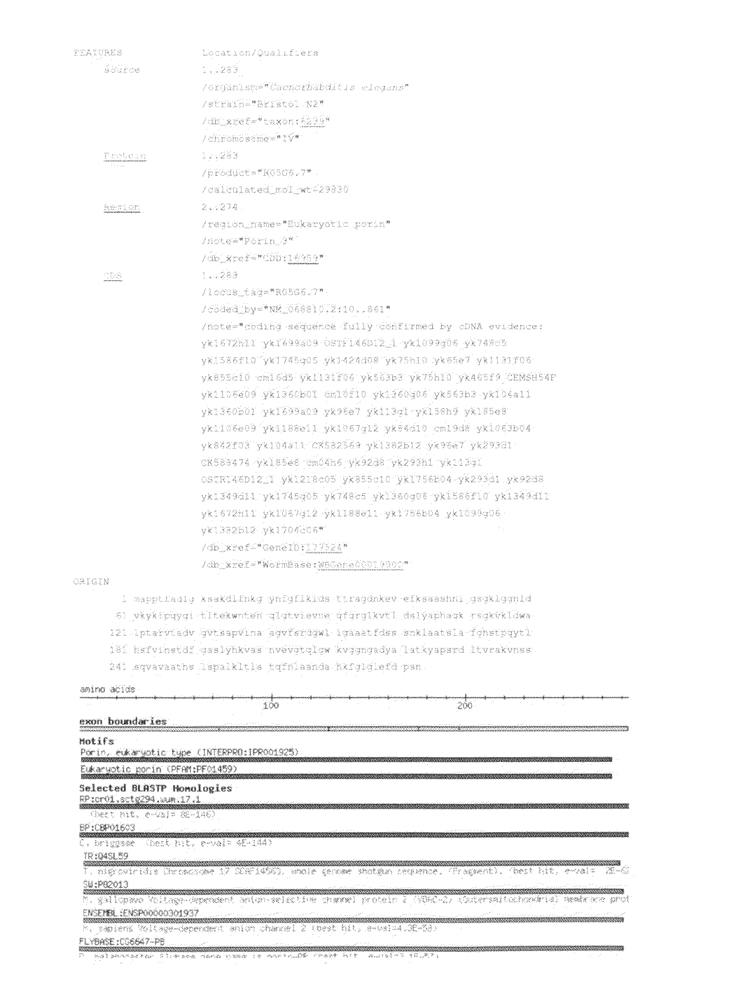

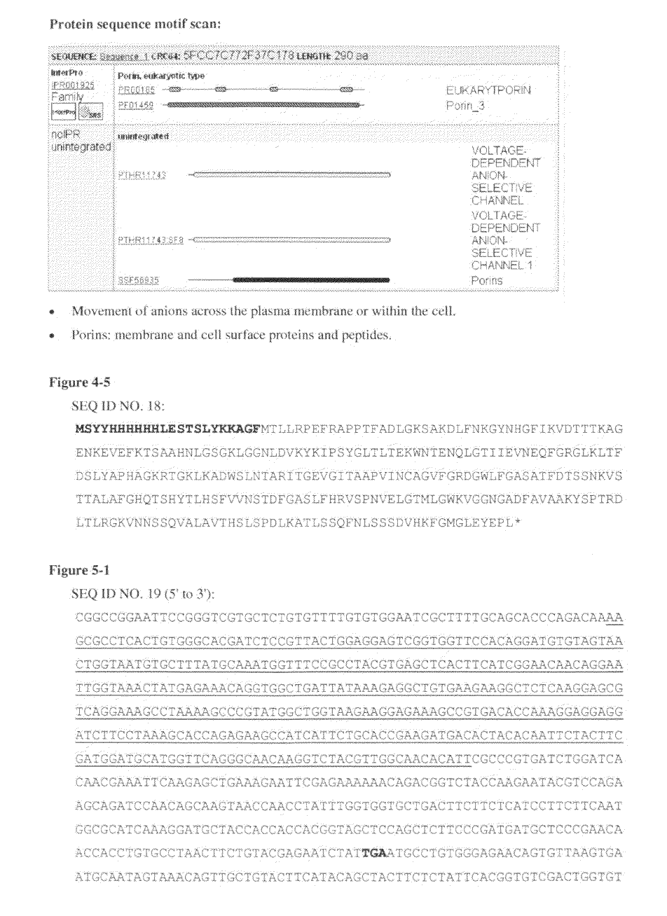

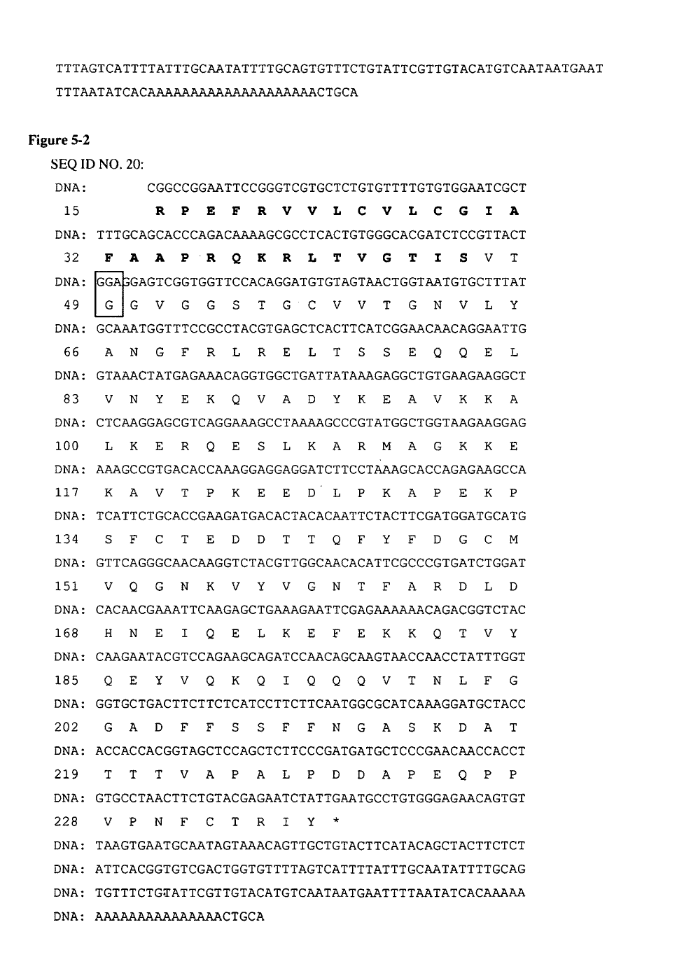

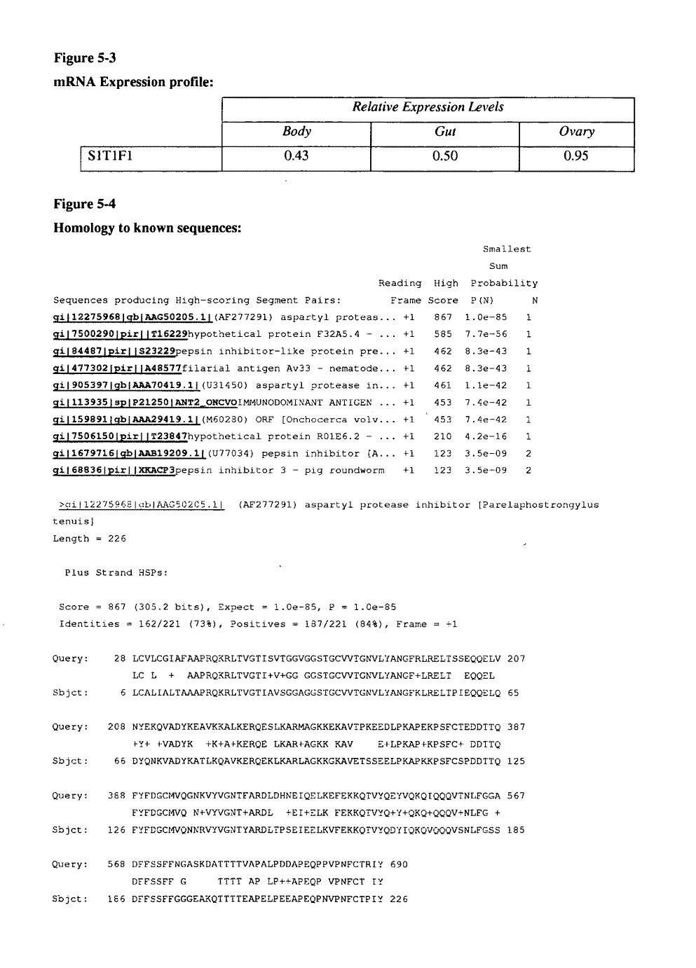

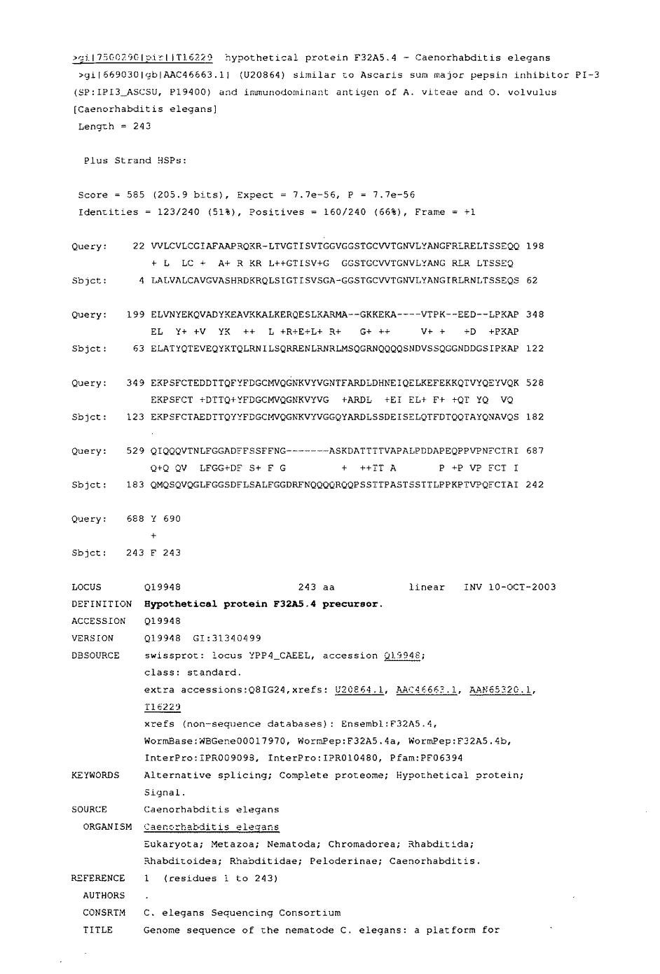

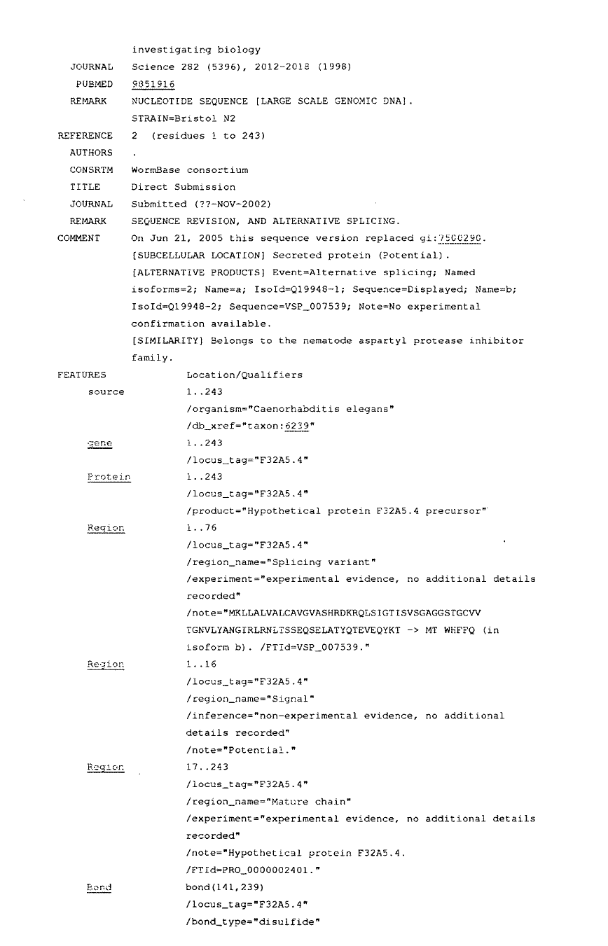

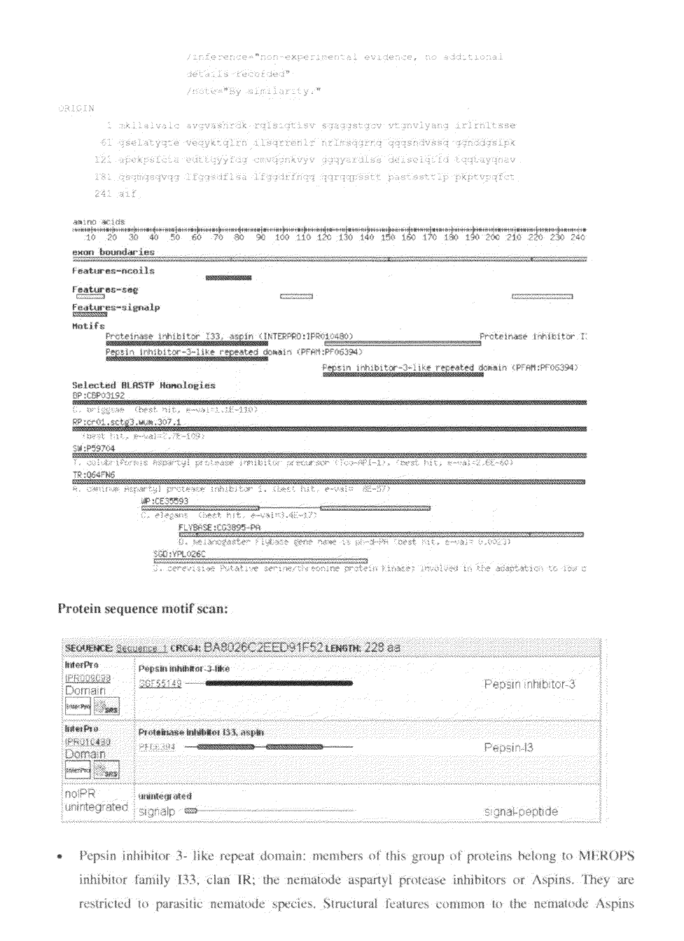

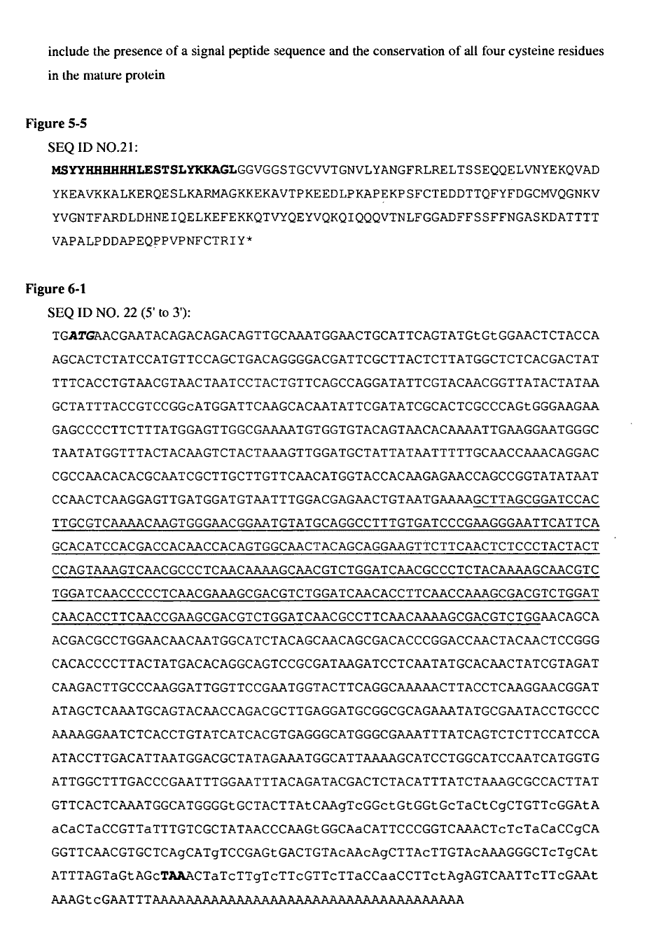

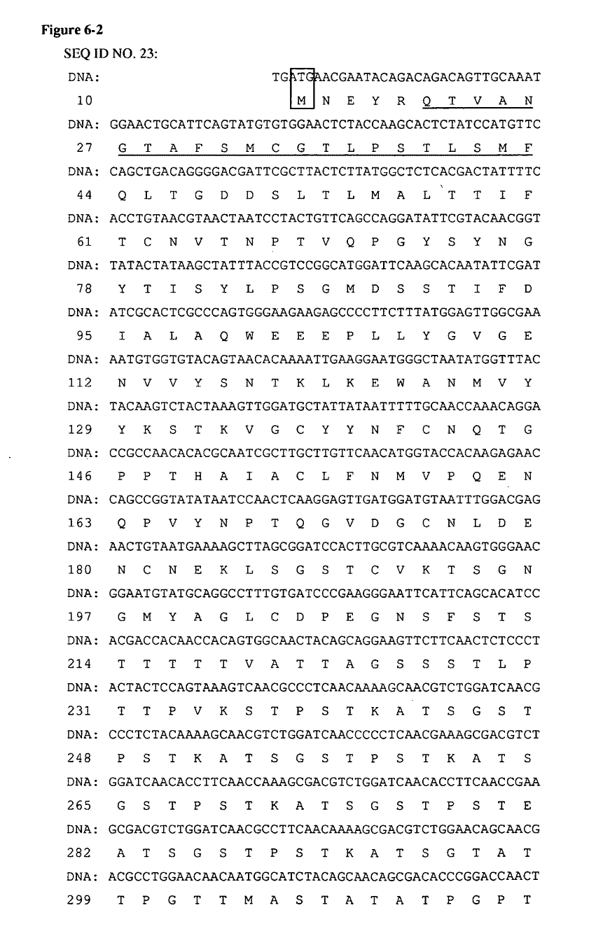

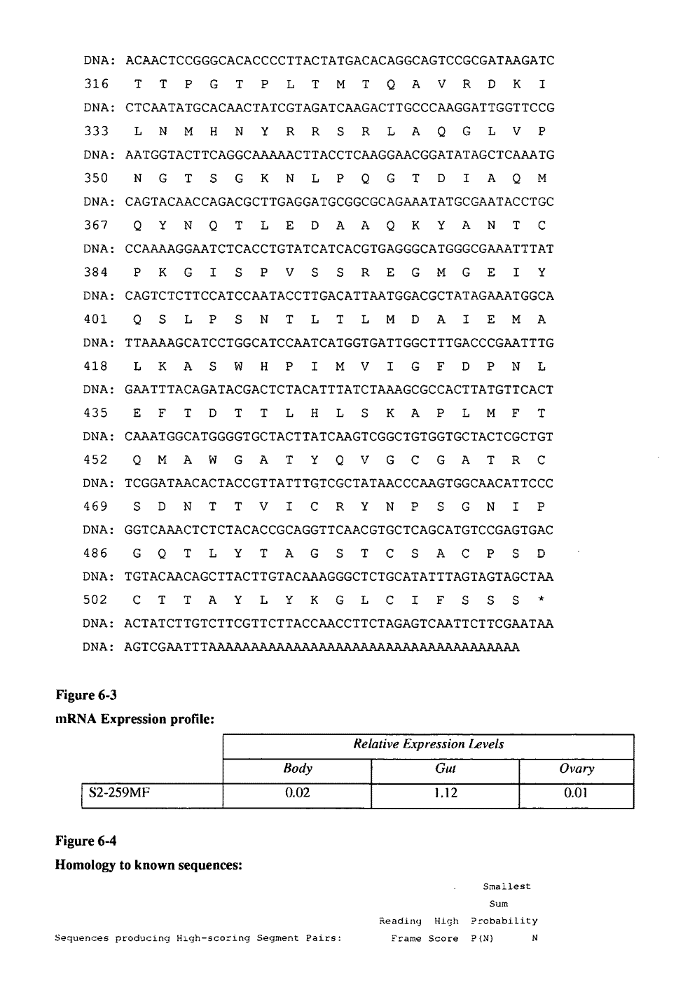

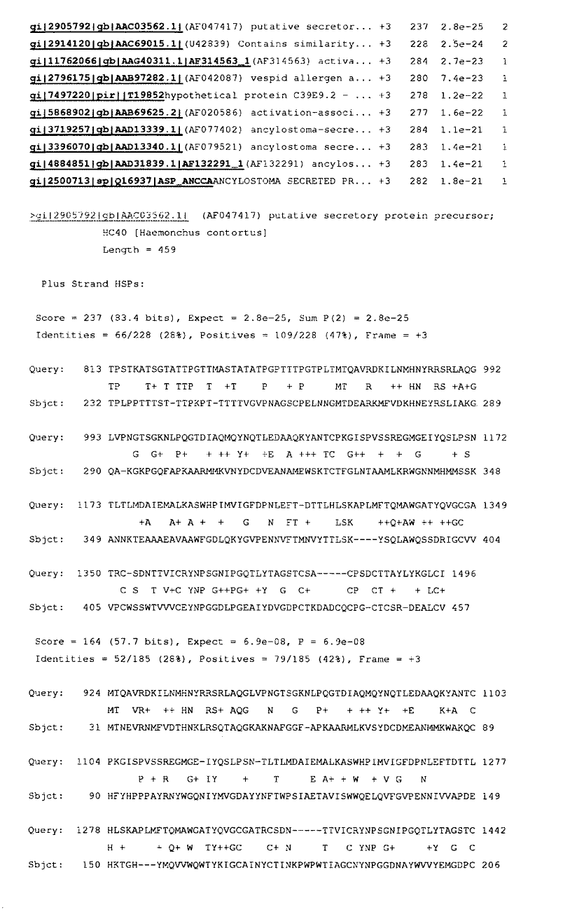

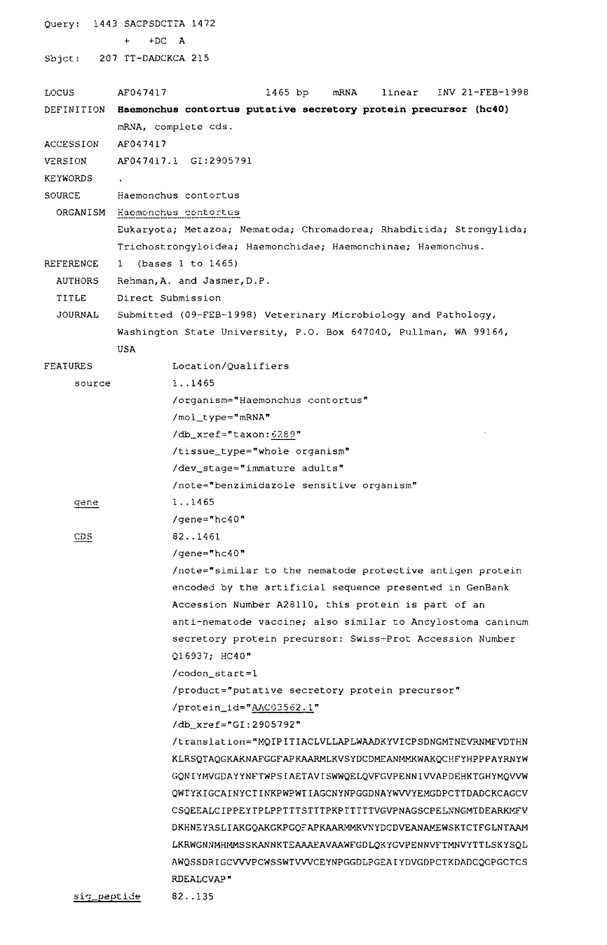

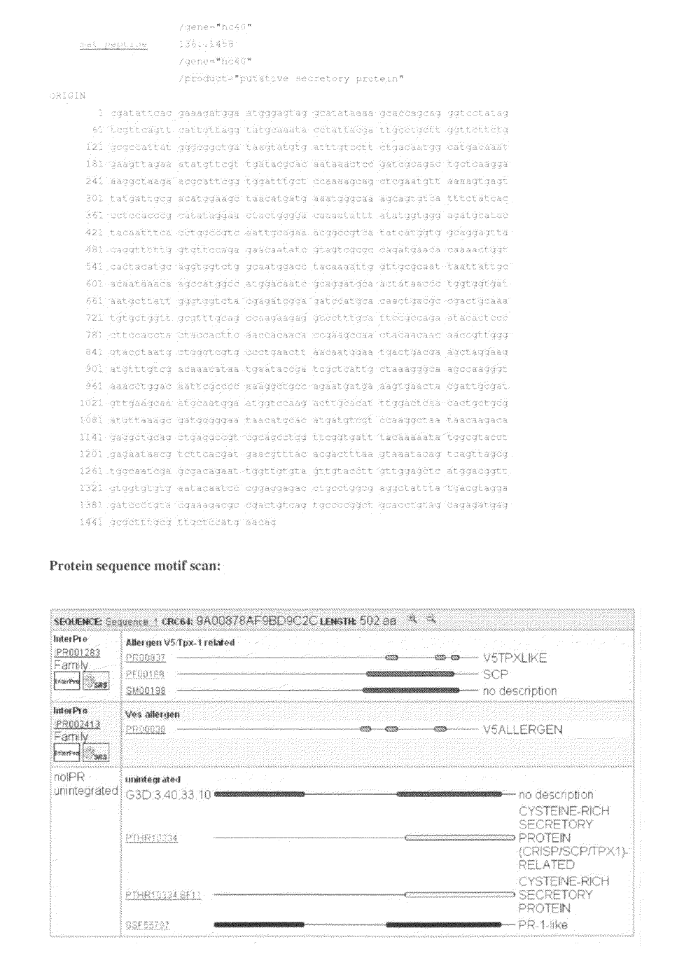

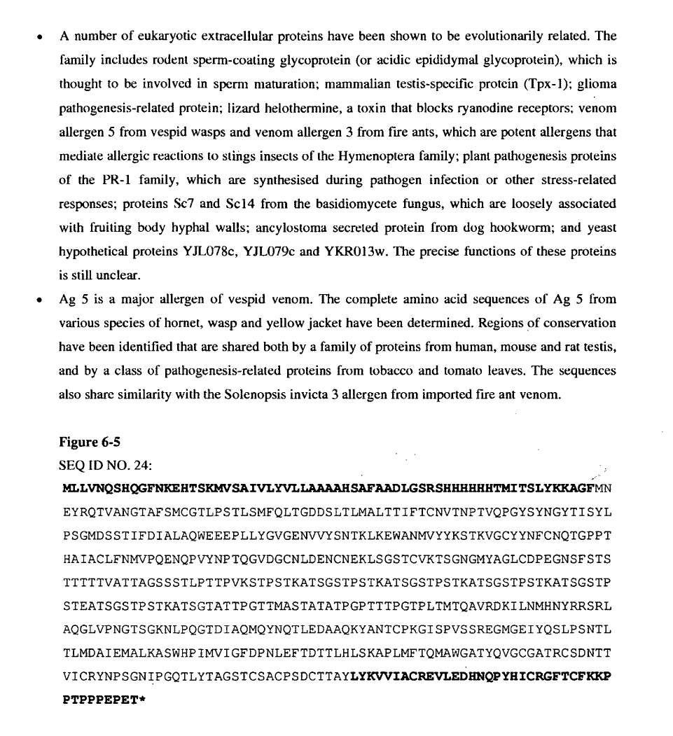

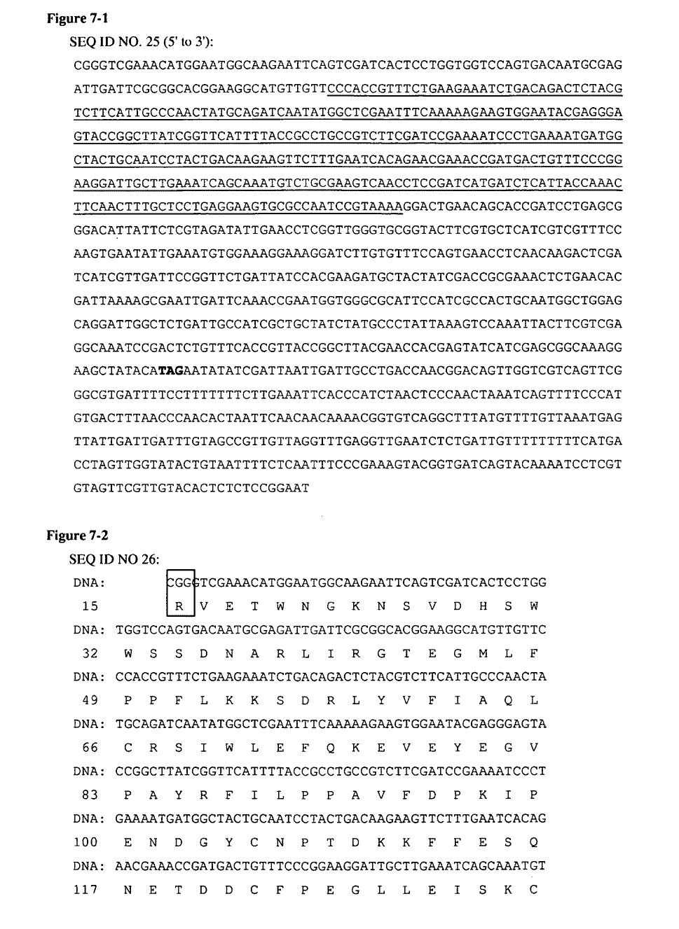

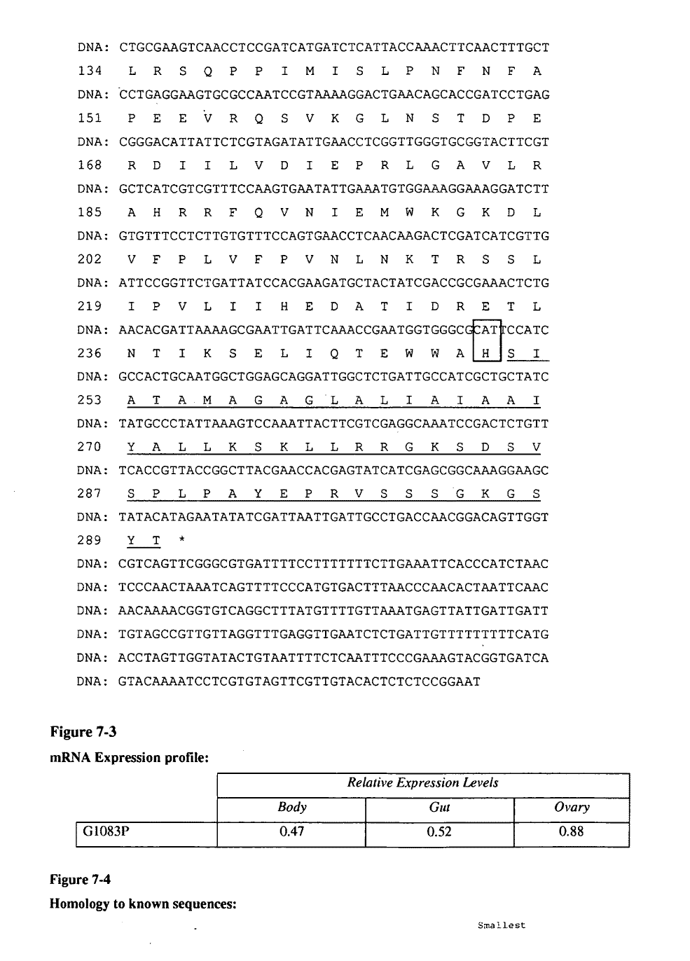

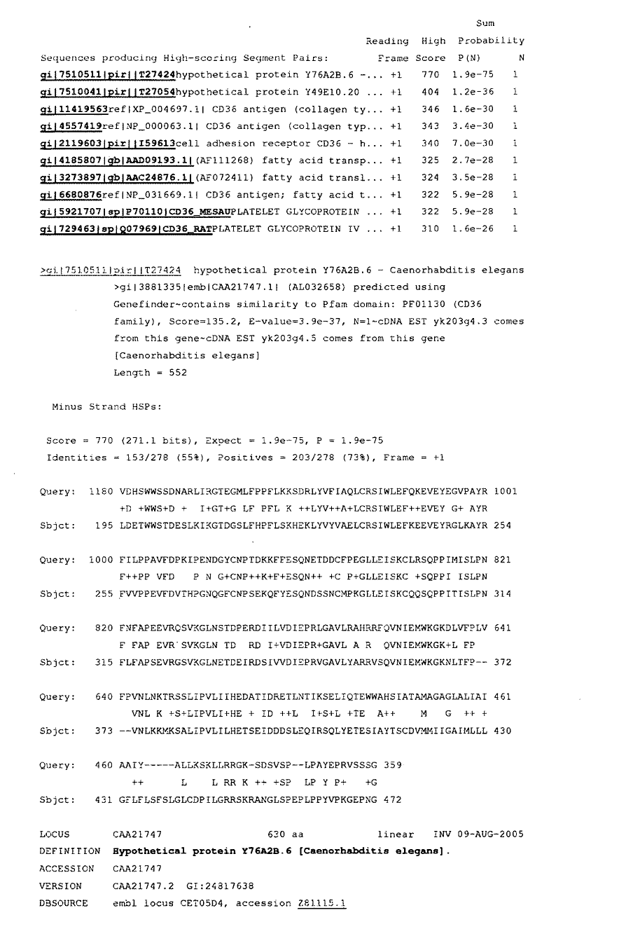

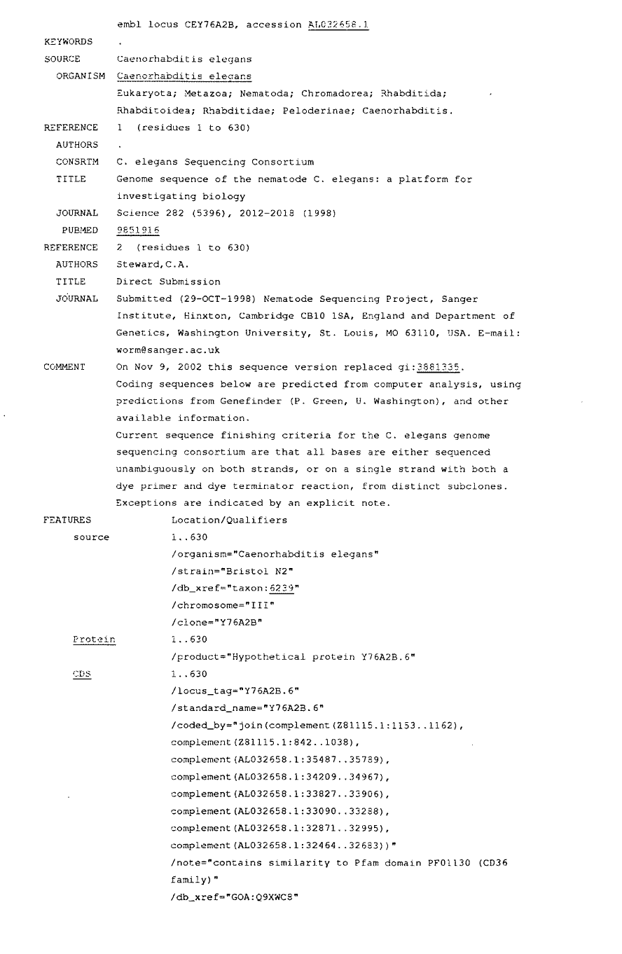

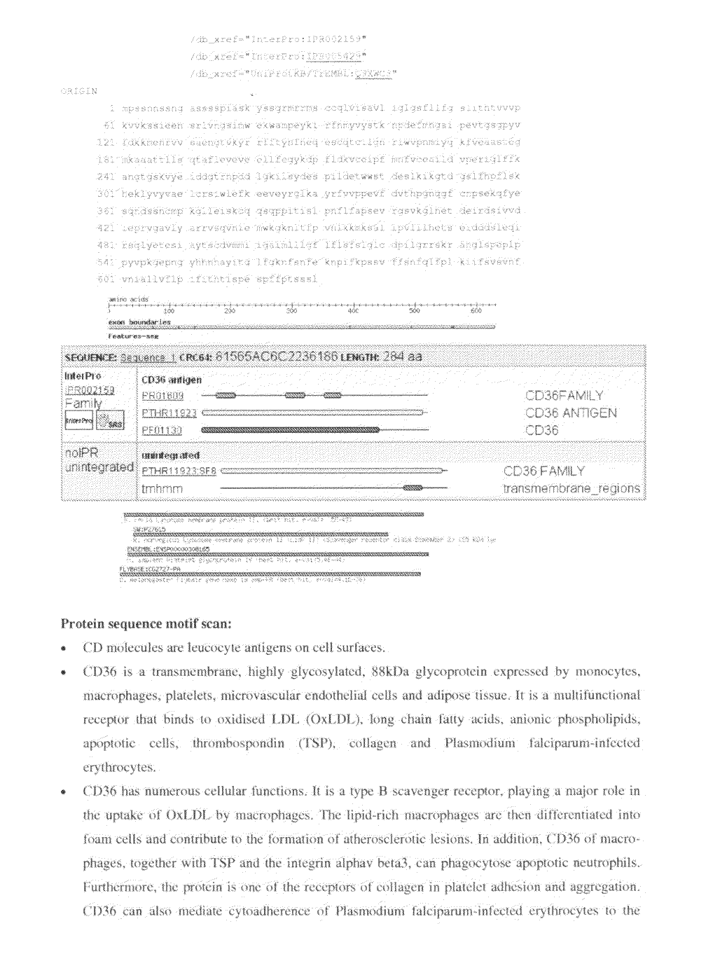

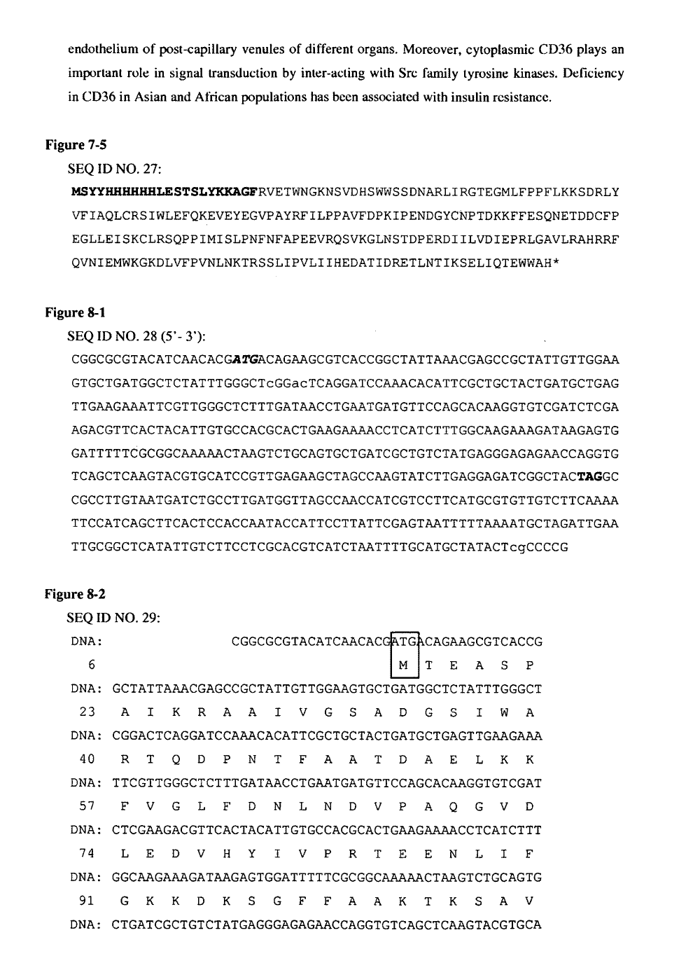

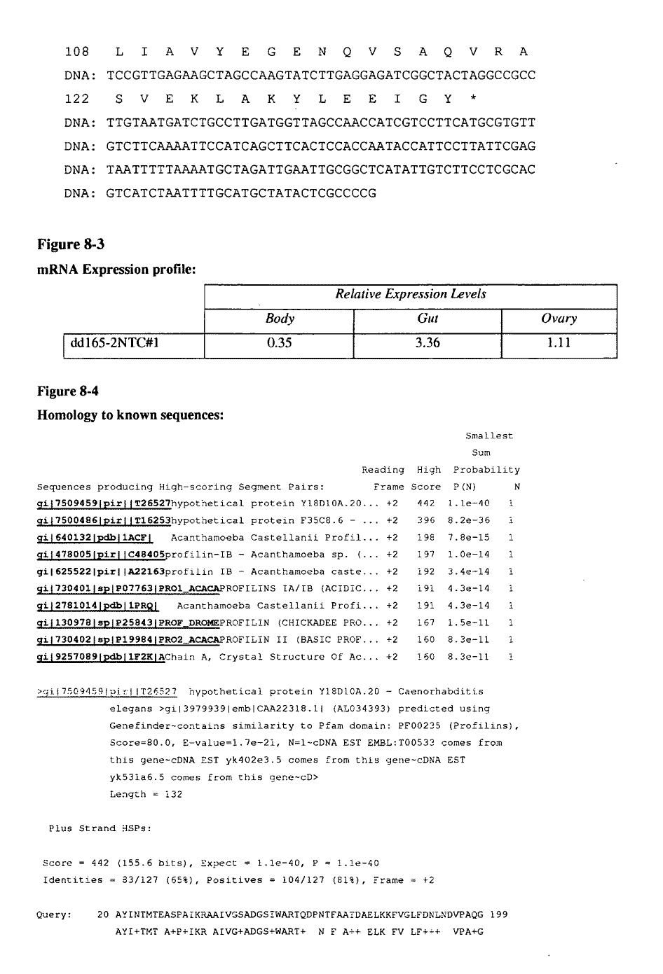

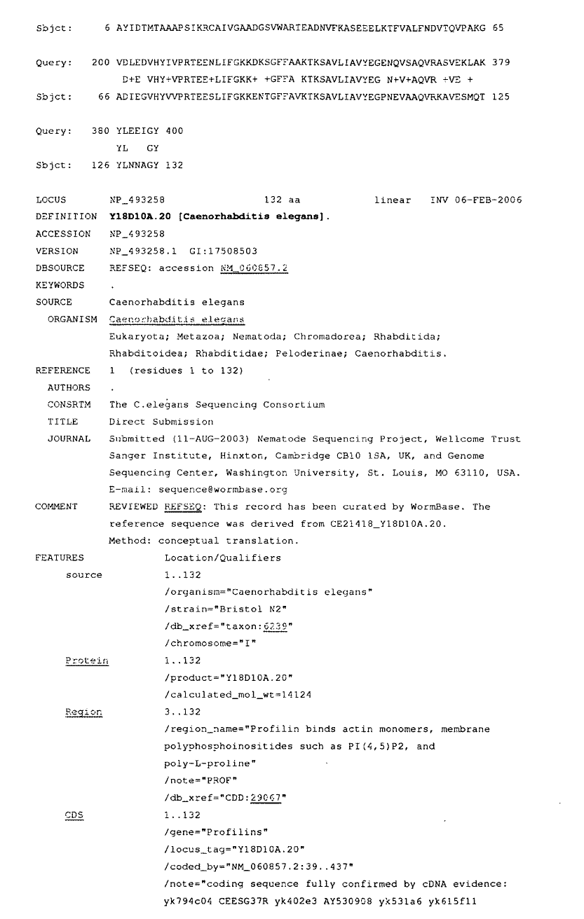

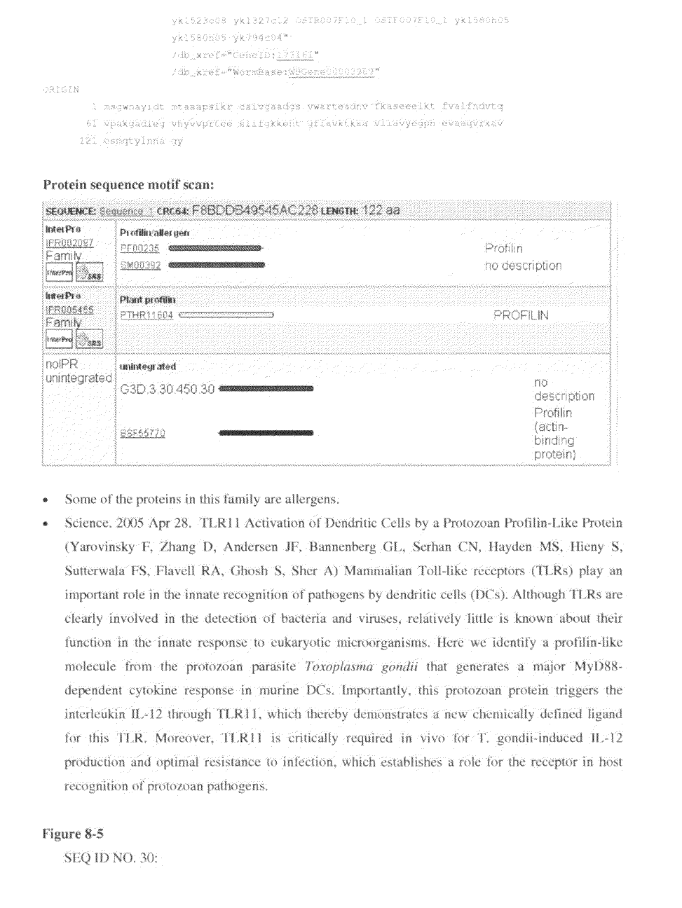

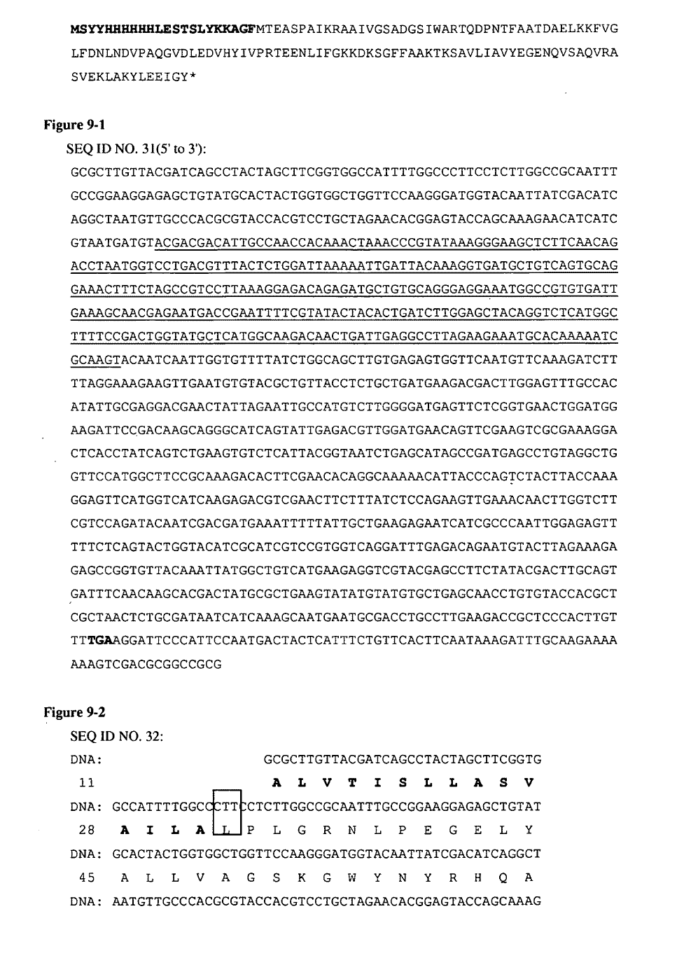

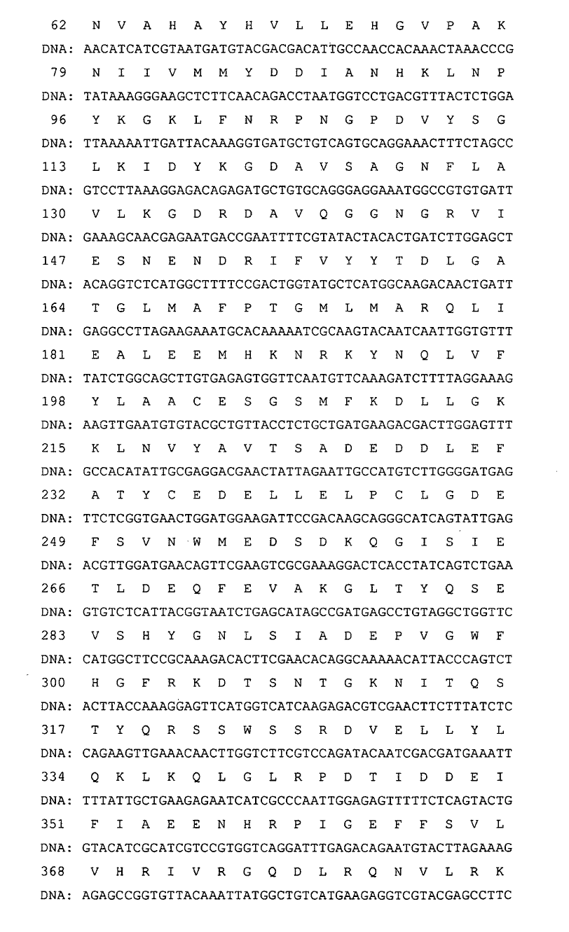

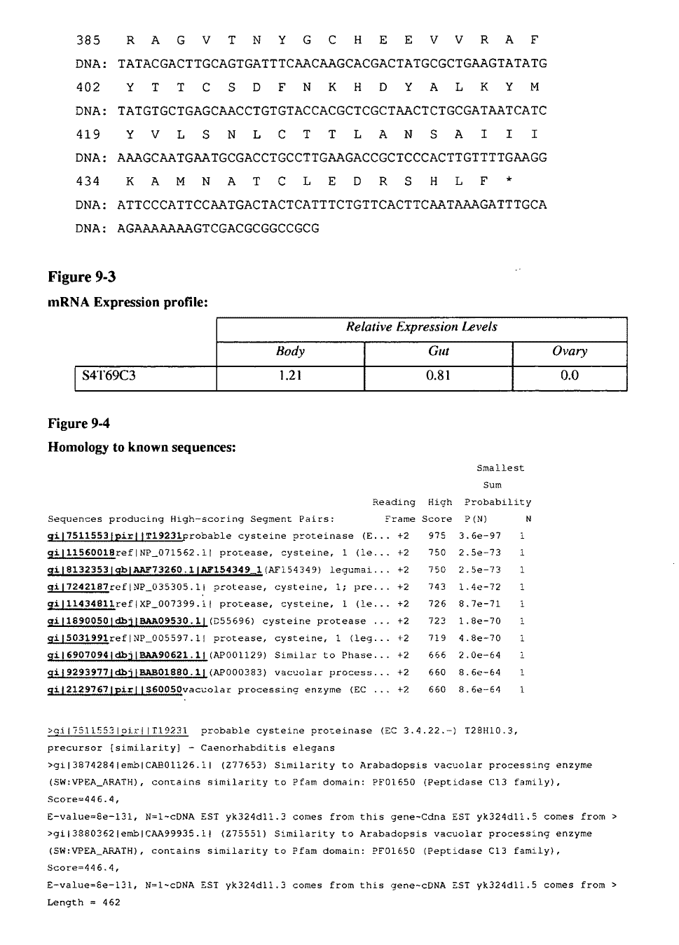

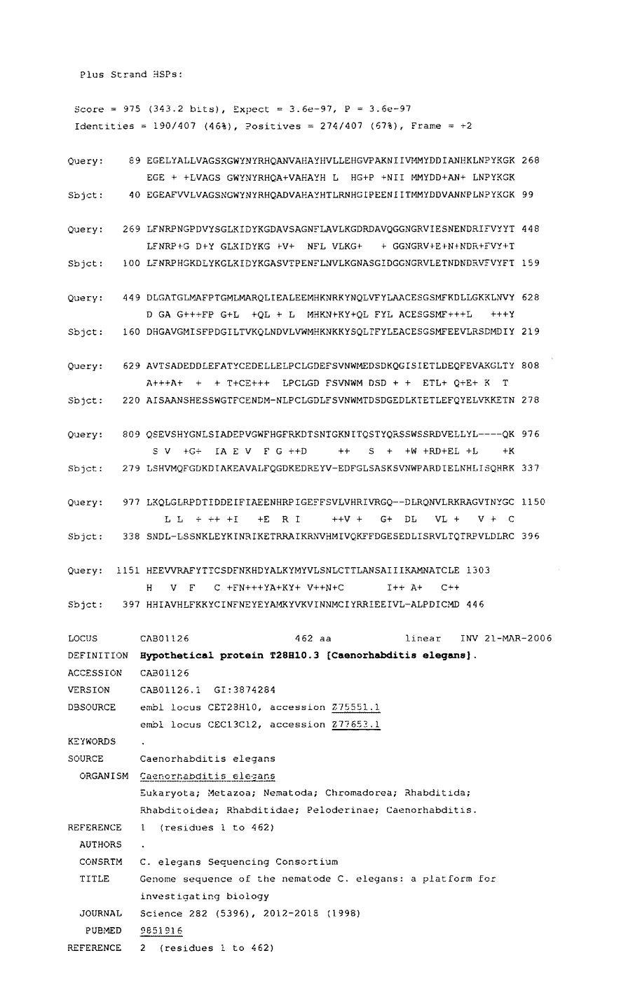

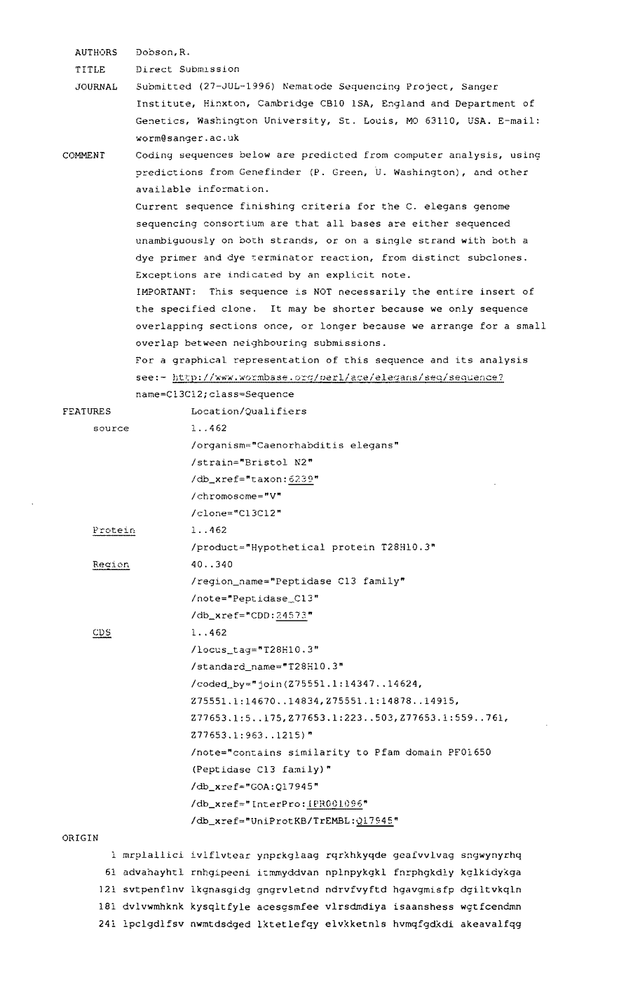

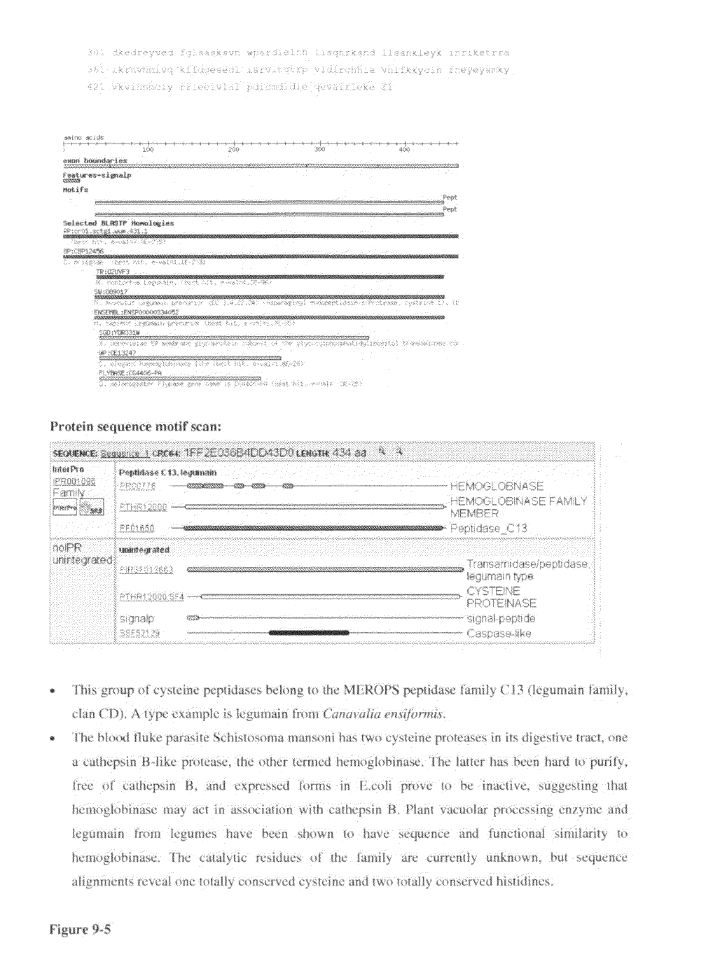

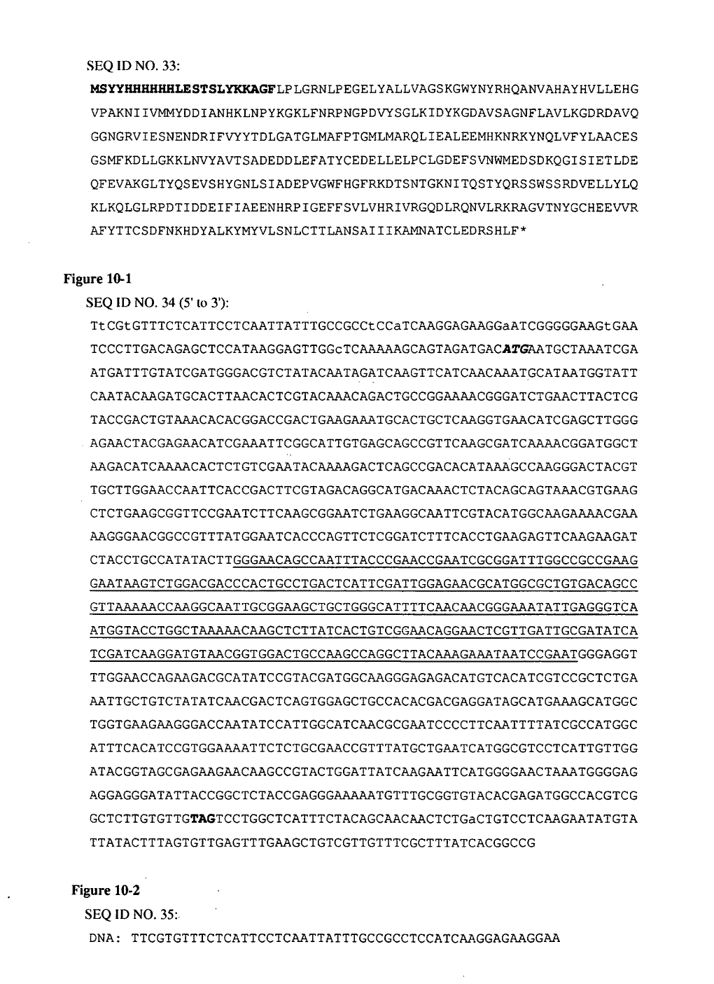

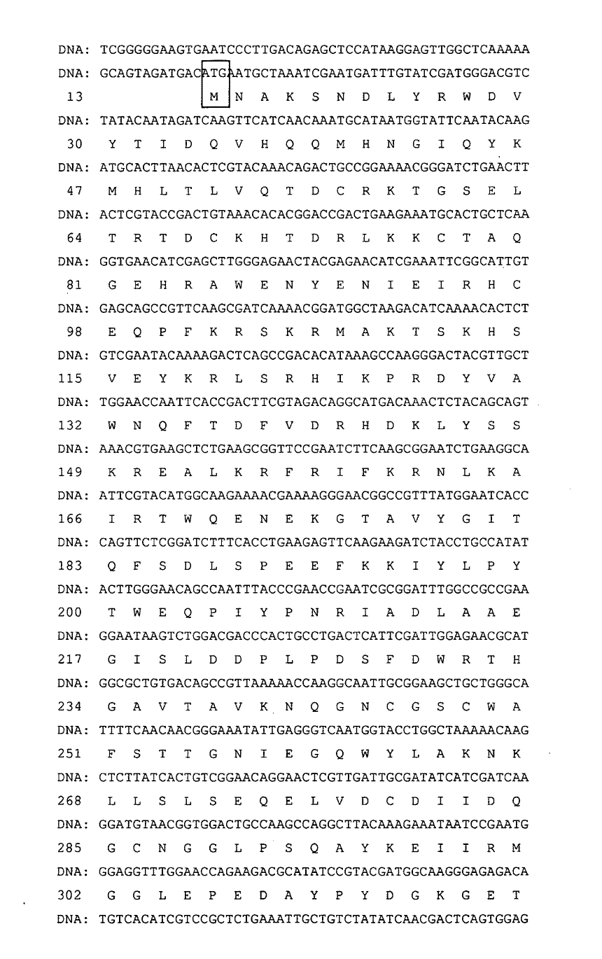

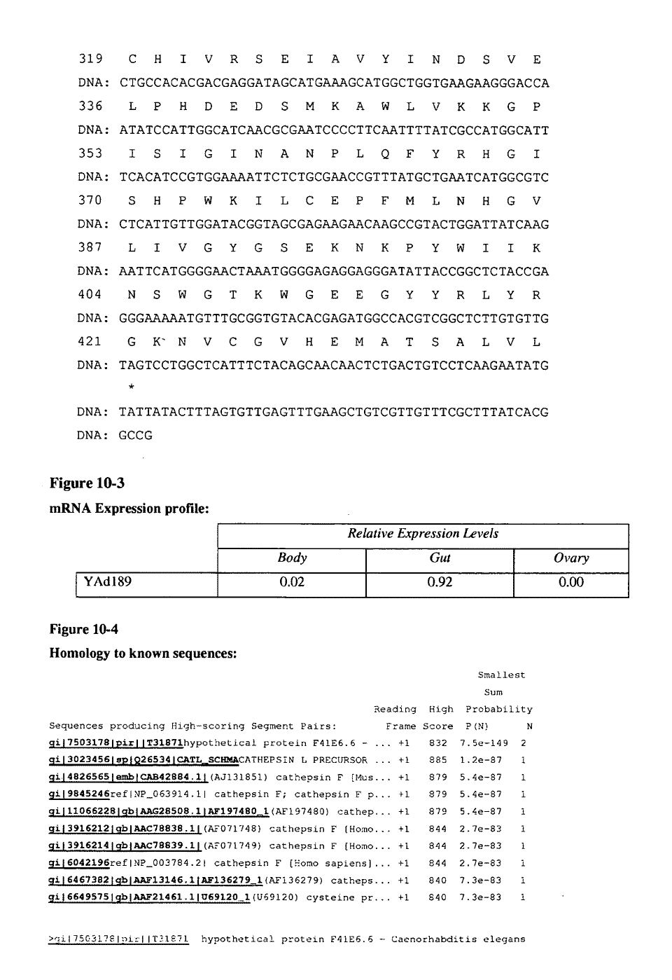

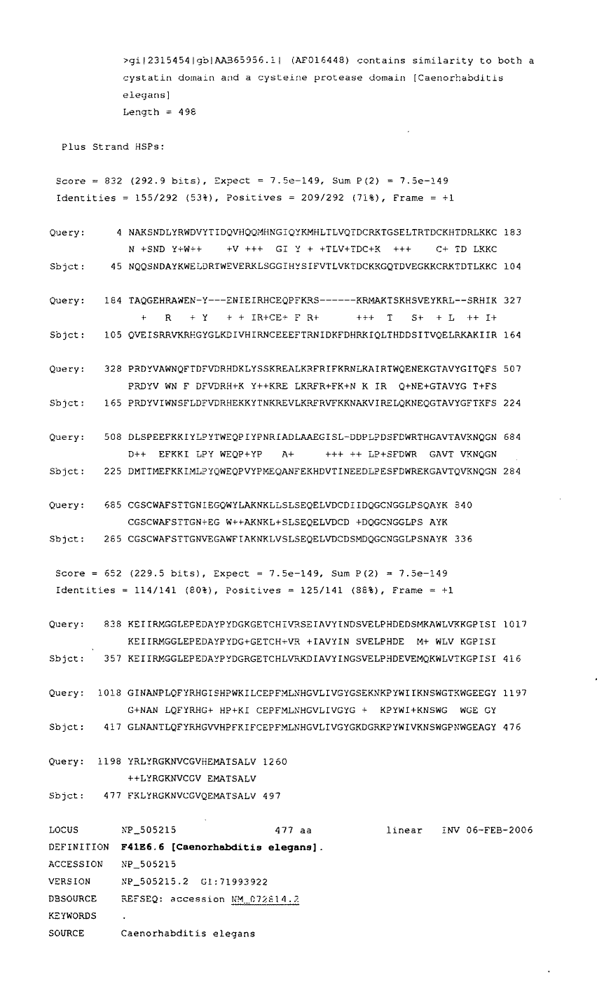

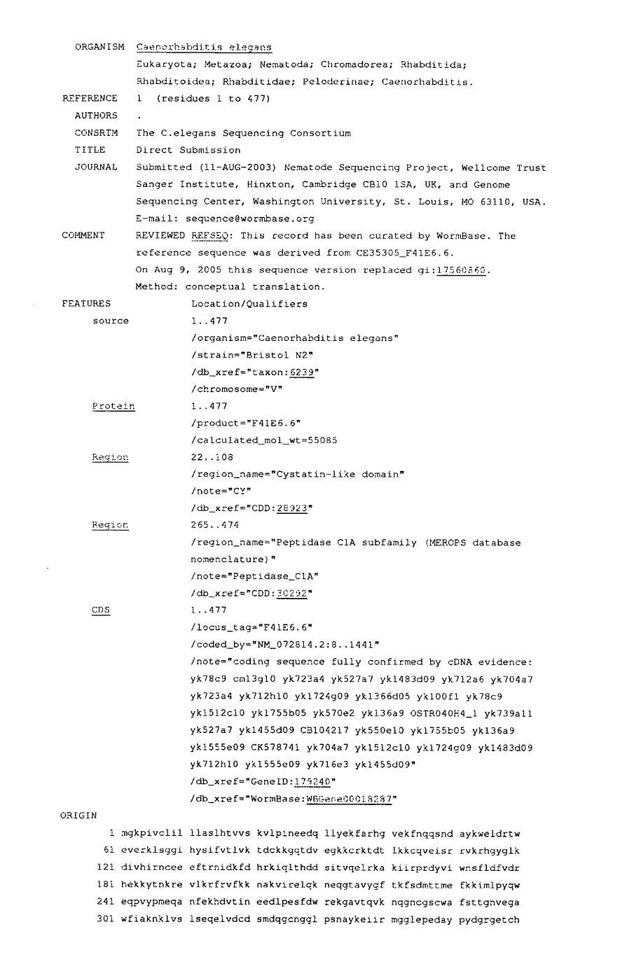

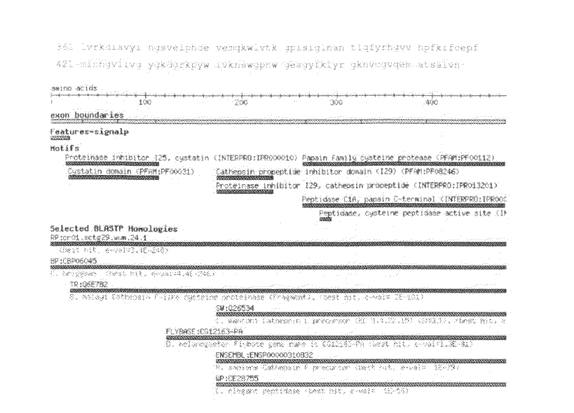

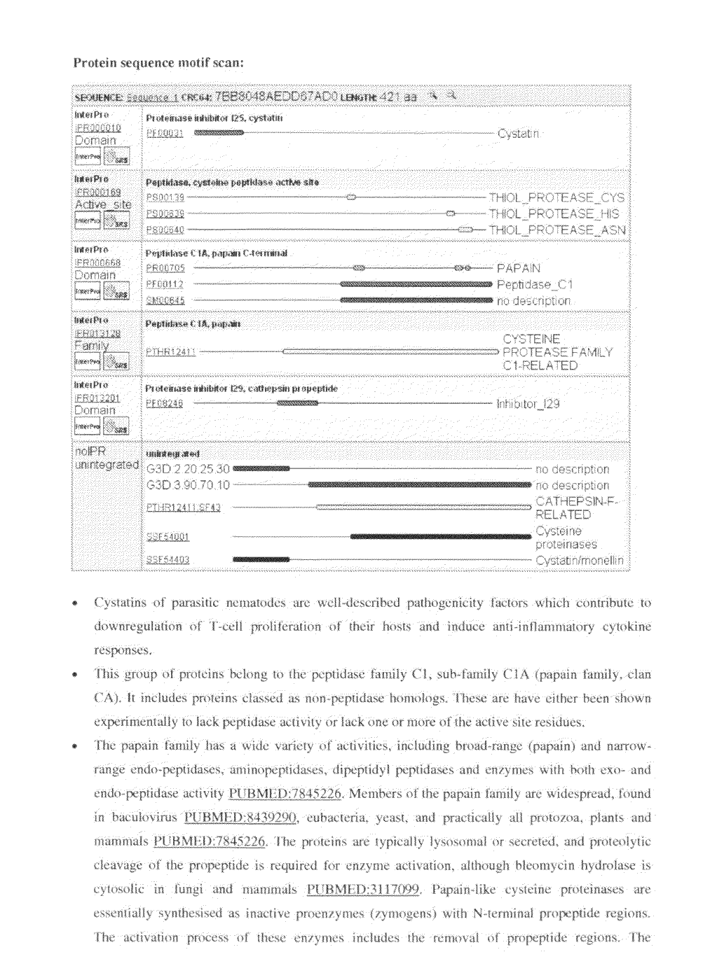

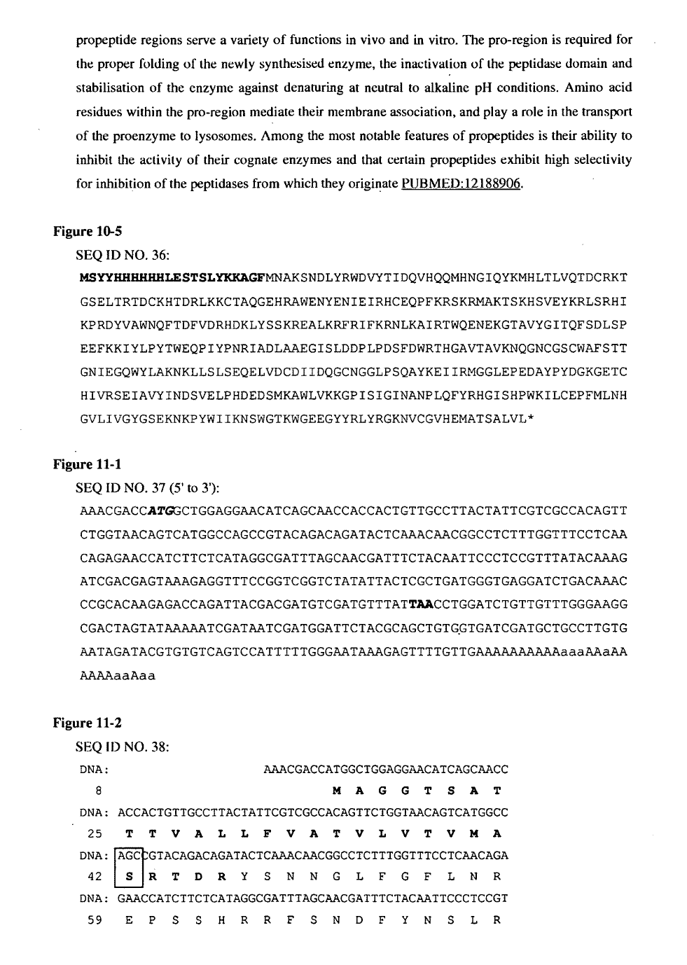

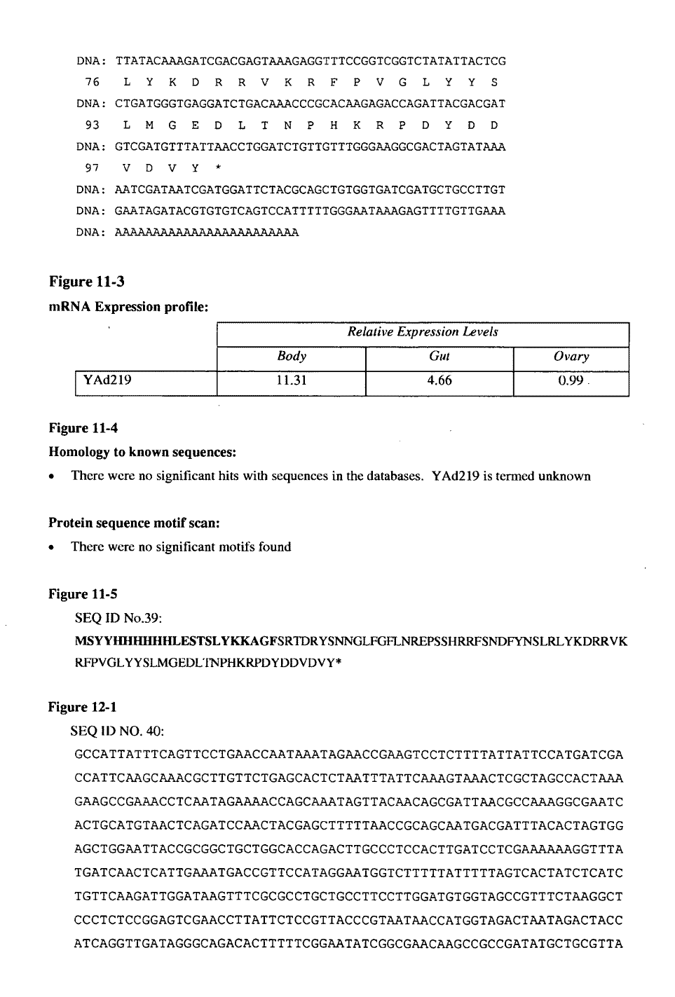

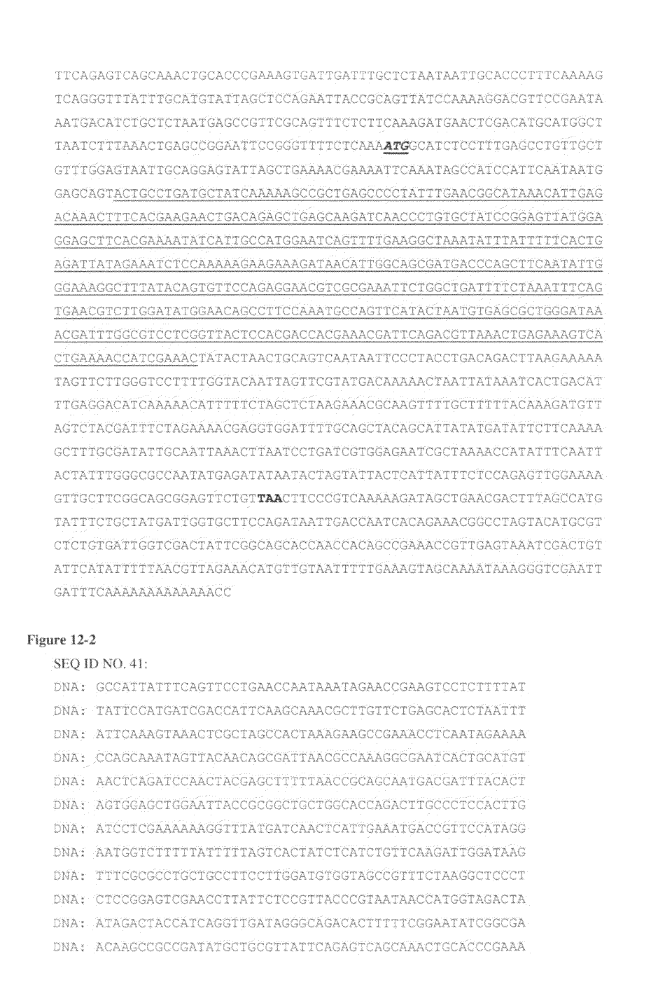

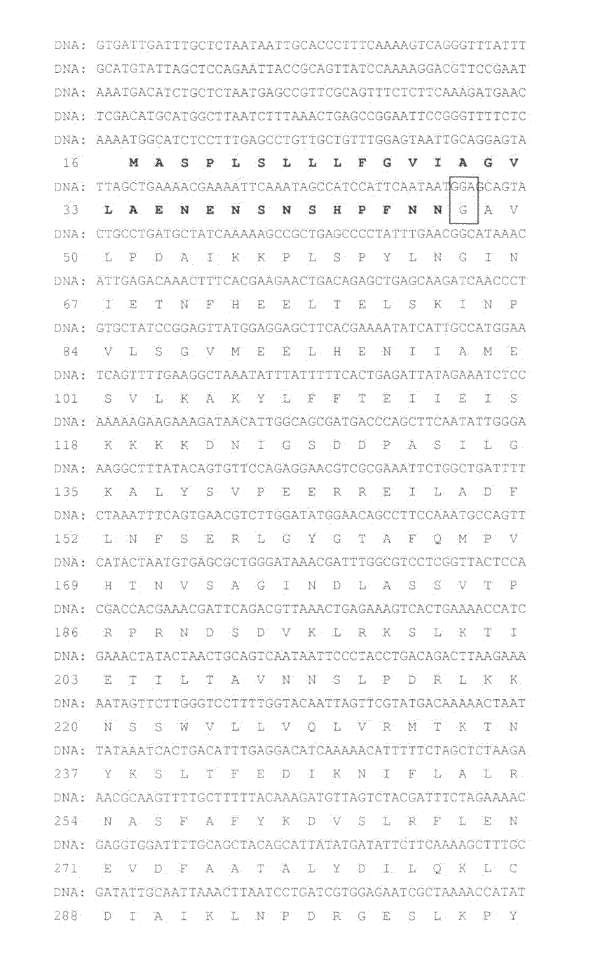

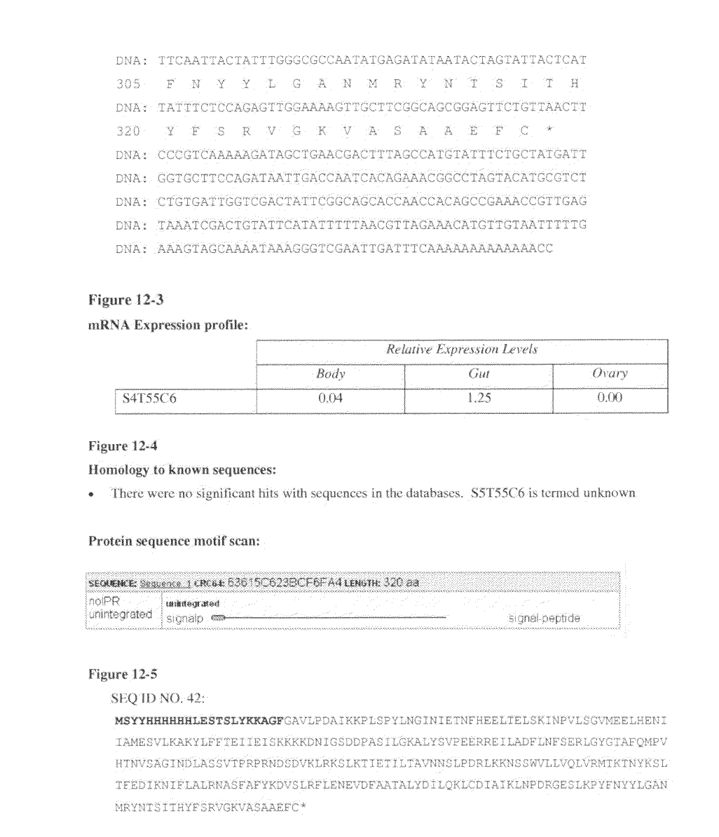

FIG. 1-2: SEQ ID No.6

An open reading frame (ORF) of 144 amino acids (aa), missing the first few residues at the N-terminus based on homology to the C. elegans hypothetical protein C14C6.2. Predicted to include some residues of a signal sequence based on homology to the C. elegans hypothetical protein C14C6.2 (shown in bold). First codon in expression clone is boxed. The nucleotide and corresponding amino acid sequence-SEQ ID No.6.

FIG. 1-3: mRNA Expression Profile

Quantitative PCR was used to determine relative expression levels of S7T104D12 in mRNA preparation from the body, gut and ovary of adult H. contortus

FIG. 1-4: Homology to known sequences and protein motif scan

FIG. 1-5: SEQ ID No.7

Heterologous expression of S7T104D12 is undertaken in E. coli using pDEST17 (Invitrogen) as the vector. The predicted recombinant protein is 161 amino acids including the initiation Methionine, His tag, and additional linker residues from pDEST17 vector sequence (shown in bold). The S7T104D12 sequence is 139 amino acids minus N-terminal residues predicted to be part of a signal sequence based on homology to the C. elegans hypothetical protein C14C6.2. The corresponding amino acid sequence is SEQ ID NO.7.

FIG. 2: Clone S4T58F9

FIG. 2-1: SEQ ID No. 8

mRNA is isolated from eL4bf (tester) and xL3 (driver) parasitic preparations. The mRNA preparations are subjected to the subtractive suppressive hybridisation method. Unhybridised cDNA molecules are cloned into pGEMT-Easy (Promega). This is termed the S4 subtracted cDNA library (eL4bf minus xL3). The S4 subtracted library is propagated using JM109 E. coli cells. Individual colonies are selected, grown in LB-broth under the selection of ampicillin, and plasmid DNA extracted using plasmid DNA prep columns (Qiagen). The plasmid DNA is checked for purity and the presence of a cDNA insert using a spectrophotometer and restriction endonuclease digestion, respectively. A contig, cID0162, is assembled using ClustalW (http://www.ch.embnet.org/software/ClustalW.html) using the sequence obtained for the subtracted library clone S4T58F9 and Genbank sequences (GenBG734183, GenBM139343, GenCA869500, GenCA870167, GenCA957981, and GenCA958005) from H. contortus. The contig sequence contains 6 GenBank sequences and 21 subtracted library clone sequences including S4T58F9 (shown underlined). 977 by cDNA insert, including a start and stop codons (shown in bold/italics and bold respectively), with the following nucleotide sequence SEQ ID NO. 8 (5′ to 3′).

FIG. 2-2: SEQ ID No.9

An ORF of 302 aa. Predicted transmembrane domain shown underlined. First codon in expression clone is boxed. The nucleotide and corresponding aa sequence is SEQ ID NO. 9.

FIG. 2-3: mRNA Expression Profile

Quantitative PCR was used to determine relative expression levels of S4T58F9 in mRNA preparation from the body, gut and ovary of adult H. contortus

FIG. 2-4: Homology to known sequences and protein motif scan

FIG. 2-5: SEQ ID No.10

Heterologous expression of the contig sequence (cID0162) is undertaken in E. coli using pDEST17 (Invitrogen) as the vector. The predicted recombinant protein is 287 aa, including the initiation Methionine, His tag, and additional linker residues from pDEST17 vector sequence (shown in bold). The contig sequence (cID0162) sequence is 265 aa, minus N-terminal residues predicted to be part of a transmembrane domain. The corresponding amino acid sequence is SEQ ID NO 10.

FIG. 3—Clone S1-381

FIG. 3-1: SEQ ID No.12

mRNA is isolated from eL4 (tester) and xL3 (driver) parasitic preparations. The mRNA preparations are subjected to the subtractive suppressive hybridisation method. Unhybridised cDNA molecules are cloned into pGEMT-Easy (Promega). This is termed the S1 subtracted cDNA library (eL4 minus xL3). The S1 subtracted library is propagated using JM109 E. coli cells. Individual colonies are selected, grown in LB-broth under the selection of ampicillin, and plasmid DNA extracted using plasmid DNA prep columns (Qiagen). The plasmid DNA is checked for purity and the presence of a cDNA insert using a spectrophotometer and restriction endonuclease digestion, respectively. A H. contortus cDNA library is screened using S1-381 as a probe. The screening procedure is repeated to obtain well-isolated positive plaques (ie primary and secondary screens). A number of positive cDNA clones hybridised to the probe; these are excised into pBluescript (Stratagene) for sequence verification.

The vector sequencing primer sites are used to sequence the pBluescript cDNA clone. The flanking vector primers 17 (SEQ ID NO.3, GTAATACGACTCACTATAG) and T3 (SEQ ID NO.11, CAATTAACCCTCACTAAAG) are used. Clone S1-381#2 is found to contain a 846 by cDNA insert, including a stop codon (shown in bold) but no start codon, the sequence for S1-381 subtracted library probe is shown underlined, with the nucleotide sequence: SEQ ID NO. 12 (5′ to 3′).

FIG. 3-2: SEQ ID No.13

An ORF of 167 aa, missing the first few residues at the N-terminus based on homology to the C. elegans hypothetical protein C07E3.9. Predicted to include some residues of a signal sequence based on homology to the C. elegans hypothetical protein C07E3.9 (shown in bold). First codon in expression clone is boxed. The nucleotide and corresponding amino acid sequence SEQ ID No. 13.

FIG. 3-3: mRNA Expression Profile

Quantitative PCR was used to determine relative expression levels of S1-381 in mRNA preparation from the body, gut and ovary of adult H. contortus

FIG. 3-4: Homology to known sequences and protein motif scan

FIG. 3-5: SEQ ID No. 14

Heterologous expression of the cDNA clone S1-381 is undertaken in E. coli using pDEST17 (Invitrogen) as the vector. The predicted recombinant protein is 168 aa, including the initiation Methionine, His tag, and additional linker residues from pDEST17 vector sequence (shown in bold). The S1-381 sequence is 146 aa, minus minus N-terminal residues predicted to be part of a signal sequence based on homology to the C. elegans hypothetical protein C07E3.9. The corresponding amino acid sequence is SEQ ID NO. 14.

FIG. 4—Clone G0142

FIG. 4-1: SEQ ID No.16

From an adult gut H. contortus cDNA. An adult gut H. contortus cDNA library is screened using G0142 as a probe. The screening procedure is repeated to obtain well-isolated positive plaques (ie primary and secondary screens). A number of positive cDNA clones hybridised to the probe; these are excised into pTriplEx (Clonetech) for sequence verification. The vector sequencing primer sites are used to sequence the pTriplEx cDNA library clone. The flanking vector primers T7 (SEQ ID NO.3, GTAATACGACTCACTATAG) and λTriplEx5′ (SEQ ID NO.15, TCCGAGATCTGGACGAGC) are used. Clone G0142-1 is found to contain: a 1788 by cDNA insert, including a start and stop codons (shown in bold/italics and bold respectively), sequence for the G0142 probe is shown underlined, with the following nucleotide sequence: SEQ ID NO. 16 (5′ to 3′).

FIG. 4-2: SEQ ID No.17

An ORF of 290 amino acids. First codon in expression clone is boxed. The nucleotide and corresponding amino acid sequence: SEQ ID NO. 17.

FIG. 4-3: mRNA Expression Profile

Quantitative PCR was used to determine relative expression levels of G0142 in mRNA preparation from the body, gut and ovary of adult H. contortus

FIG. 4-4: Homology to known sequences and protein motif scan

FIG. 4-5: SEC) ID No. 18

Heterologous expression of G0412 is undertaken in E. coli using pDEST17 (Invitrogen) as the vector. The predicted recombinant protein is 312 aa, including the initiation Methionine, His tag, and additional linker residues from pDEST17 vector sequence (shown in bold). The G0142-1 sequence is 290 aa. The corresponding amino acid sequence is SEQ ID NO. 18.

FIG. 5: Clone S1T1 F1

FIG. 5-1: SEQ ID No. 19

mRNA is isolated from eL4 (tester) and xL3 (driver) parasitic preparations. The mRNA preparations are subjected to the subtractive suppressive hybridisation method. Unhybridised cDNA molecules are cloned into pGEMT-Easy (Promega). This is termed the S1 subtracted cDNA library (eL4 minus xL3). The S1 subtracted library is propagated using JM109 E. coli cells. Individual colonies are selected, grown in LB-broth under the selection of ampicillin, and plasmid DNA extracted using plasmid DNA prep columns (Qiagen). The plasmid DNA is checked for purity and the presence of a cDNA insert using a spectrophotometer and restriction endonuclease digestion, respectively. A What library is this?H. contortus cDNA library is screened using S1T1F1 as a probe. The screening procedure is repeated to obtain well-isolated positive plaques (ie primary and secondary screens). A number of positive cDNA clones hybridised to the probe; these are excised into pBluescript (Stratagene) for sequence verification.

The vector sequencing primer sites are used to sequence the pBluescript cDNA library clone. The flanking vector primers T7 (SEQ ID NO.3, GTAATACGACTCACTATAG) and T3 (SEQ ID NO.11, CAATTAACCCTCACTAAAG) are used. Clone S1T1F1#9 is found to contain:A 881 by cDNA insert, including a stop codon (shown in bold) but no start codon, sequence for the S1T1F1 probe is shown underlined, with the following nucleotide sequence: SEQ ID NO. 19 (5′ to 3′).

FIG. 5-2: SEQ ID No. 20

An ORF of 228 aa, missing the first few residues at the N-terminus based on homology to the C. elegans hypothetical protein F32A5.4. Predicted to include some residues of a signal sequence (shown in bold). First codon in expression clone is boxed. The nucleotide and corresponding amino acid sequence: SEQ ID NO. 20

FIG. 5-3: mRNA Expression Profile

Quantitative PCR was used to determine relative expression levels of S1T1F1 in mRNA preparation from the body, gut and ovary of adult H. contortus

FIG. 5-4: Homology to known sequences and protein motif scan

FIG. 5-5: SEQ ID No. 21

Heterologous expression of S1T1F1 is undertaken in E. coli using pDEST17 (Invitrogen) as the vector. The predicted recombinant protein is 218 aa, including the initiation Methionine, His tag, and additional linker residues from pDEST17 vector sequence (shown in bold). The S1T1F1#9 sequence is 196 aa minus N-terminal residues predicted to be part of a signal sequence based on homology to the C. elegans hypothetical protein F32A5.4. The corresponding amino acid sequence is SEQ ID NO.21.

FIG. 6: Clone S2-259MF

FIG. 6-1: SEQ ID No. 22

mRNA is isolated from the gut of adult females (tester) and xL3 (driver) parasitic preparations. The mRNA preparations are subjected to the subtractive suppressive hybridisation method. Unhybridised cDNA molecules are cloned into pGEMT-Easy (Promega). This is termed the S2 subtracted cDNA library (eL4 minus xL3). The S2 subtracted library is propagated using JM109 E. coli cells. Individual colonies are selected, grown in LB-broth under the selection of ampicillin, and plasmid DNA extracted using plasmid DNA prep columns (Qiagen). The plasmid DNA is checked for purity and the presence of a cDNA insert using a spectrophotometer and restriction endonuclease digestion, respectively. An adult H. contortus cDNA library is screened using S2-259MF as a probe. The screening procedure is repeated to obtain well-isolated positive plaques (ie primary and secondary screens). A number of positive cDNA clones hybridised to the probe; these are excised into pBluescript (Stratagene) for sequence verification. The vector sequencing primer sites are used to sequence the pBluescript cDNA library clone. The flanking vector primers T7 (SEQ ID NO.3, GTAATACGACTCACTATAG) and T3 (SEQ ID NO.11, CAATTAACCCTCACTAAAG) are used. Clone S2-259MF-Clone10.1 is found to contain a 1609 by cDNA insert, including the start and stop codons (bold/italics and bold respectively), sequence for the S2-259MF probe is shown underlined, with the following nucleotide sequence:

SEQ ID NO. 22 (5′ to 3′).

FIG. 6-2: SEQ ID No. 23

An ORF of 502 amino acid. TMprep predicted two possible transmembrane domains (shown underlined) the second is not as strong. TMHMM only predicted a very weak TM at the N-terminus. First codon in expression clone is boxed. The nucleotide and corresponding amino acid sequence is SEQ ID NO. 23.

FIG. 6-3: mRNA Expression Profile

Quantitative PCR was used to determine relative expression levels of S2-259MF in mRNA preparation from the body, gut and ovary of adult H. contortus

FIG. 6-4: Homology to known sequences and protein motif scan

FIG. 6-5: SEQ ID No.24

Heterologous expression of S2-259MF is undertaken in Baculovirus using pHeskVec-Destination as the vector. The predicted recombinant protein is 540 aa, including initiation Methionine, secretion signal, and His tag from pHeskVec-Destination vector sequence (shown in bold). Have an additional 40 aa at the C-terminus from the vector sequence due to an unusual recombination event during the cloning (also shown in bold). The S2-259MF-Clone10.1 sequence is 491 aa, missing 11 aa from the C-terminus due to an unusual recombination event during the cloning. The corresponding amino acid sequence is SEQ ID NO. 24.

FIG. 7: Clone G1083P

FIG. 7-1: SEQ ID No.25

cDNA clone from an adult gut H. contortus cDNA library. An adult gut H. contortus cDNA library is screened using G1083P as a probe. The screening procedure is repeated to obtain well-isolated positive plaques (ie primary and secondary screens). A number of positive cDNA clones hybridise to the probe; these are excised into pTriplEx (Clonetech) for sequence verification. The vector sequencing primer sites are used to sequence the pTriplEx cDNA library clone. The flanking vector primers T7 (SEQ ID NO.3, GTAATACGACTCACTATAG) and λTriplEx5′ (SEQ ID NO.15, TCCGAGATCTGGACGAGC) are used. Clone G1083P is found to contain:

a ˜1200 by cDNA insert, including a stop codon (shown in bold) but no start codon. The sequencing primer binds close to the cloning site so there is no complete sequence of this clone (missing ˜20-40 nucleotides at the 3′ end). The sequence for the G1083P probe is shown underlined. The corresponding nucleotide sequence is SEQ ID NO. 25 (5′ to 3′).

FIG. 7-2: SEQ ID No.26

An ORF of 289 amino acids, including a strong transmembrane domains (shown underline) predicted at the C-terminus. First and last codons in the expression clone, respectively, are shown boxed. The nucleotide and corresponding amino acid sequence is SEQ ID NO 26.

FIG. 7-3: mRNA Expression Profile

Quantitative PCR was used to determine relative expression levels of G1083P in mRNA preparation from the body, gut and ovary of adult H. contortus

FIG. 7-4: Homology to known sequences and protein motif scan

FIG. 7-5: SEQ ID No.27

Heterologous expression of G1083P is undertaken in E. coli using pDEST17 (Invitrogen) as the vector. The predicted recombinant protein is 252 amino acids, including the initiation Methionine, His tag, and additional linker residues from pDEST17 vector sequence (shown in bold). The G1083P1 sequence is 230 amino acids, minus the C-terminal predicted transmembrane domain. The corresponding amino acid sequence is SEQ ID NO. 27.

FIG. 8-Clone dd165-2NTC#1

FIG. 8-1: SEQ ID No.28

From an adult gut H. contortus cDNA library. The vector sequencing primer sites are used to sequence the pTrilplEx cDNA library clone. The flanking vector primers 17 (SEQ ID NO. 3 GTAATACGACTCACTATAG) and λTriplEx5′ (SEQ ID NO.15, TCCGAGATCTGGACGAGC) were used. Clone dd165-2NTC#1 is found to contain: a 579 by cDNA insert, including start and stop codons (shown in bold/italics and bold respectively), with the following nucleotide sequence:SEQ ID NO. 28 (5′-3′).

FIG. 8-2: SEQ ID No.29

An open reading frame (ORF) of 122 amino acids. First codon in expression clone is shown boxed. The nucleotide and corresponding amino acid sequence: SEQ ID NO. 29.

FIG. 8-3: mRNA Expression Profile

Quantitative PCR was used to determine relative expression levels of dd165-2NTC#1 in mRNA preparation from the body, gut and ovary of adult H. contortus

FIG. 8-4: Homology to known sequences and protein motif scan

FIG. 8-5: SEQ ID No.30

Heterologous expression of dd165-2NTC#1 is undertaken in E. coli using pDEST17 (Invitrogen) as the vector. The predicted recombinant protein is 144 amino acids, including the initiation Methionine, His tag, and additional linker residues from pDEST17 vector sequence (shown in bold). The dd165-2NTC#1 sequence is 122 amino acids. The corresponding amino acid sequence is SEQ ID NO. 30:

FIG. 9: Clone S4T69C3

FIG. 9-1: SEQ ID No.31

mRNA is isolated from eL4bf (tester) and xL3 (driver) parasitic preparations. The mRNA preparations are subjected to the subtractive suppressive hybridisation method. Unhybridised cDNA molecules are cloned into pGEMT-Easy (Promega). This is termed the S4 subtracted cDNA library (eL4bf minus xL3). The S4 subtracted library is propagated using JM109 E. coli cells. Individual colonies are selected, grown in LB-broth under the selection of ampicillin, and plasmid DNA extracted using plasmid DNA prep columns (Qiagen). The plasmid DNA is checked for purity and the presence of a cDNA insert using a spectrophotometer and restriction endonuclease digestion, respectively. An eL4bf H. contortus cDNA library is screened using a radiolabelled S4T69C3 fragment. The screening procedure is repeated to obtain well-isolated positive plaques (ie primary and secondary screens). A number of positive cDNA clones hybridised to the probe; these are excised into pBluescript (Stratagene) for sequence verification. The vector sequencing primer sites are used to sequence the pBluescript cDNA library clone. The flanking vector primers T7 (SEQ ID NO.3, GTAATACGACTCACTATAG) and T3 (SEQ ID NO.11, CAATTAACCCTCACTAAAG) are used. Clone C69C3-5.5 is found to contain: a 1383 by cDNA insert, including a stop codon (shown in bold) but no start codon, sequence for the S4T69C3 probe is shown underlined, with the following nucleotide sequence:SEQ ID NO. 31 (5′ to 3′).

FIG. 9-2: SEQ ID No.32

An ORF of 434 amino acids missing the first few amino acid residues at the N-terminus based on homology to C. elegans hypothetical protein T28H10.3. Predicted to include some residues of a signal sequence based on homology to C. elegans hypothetical protein T28H10.3 (in bold) and SignalP prediction. First codon in expression clone is boxed. The corresponding amino acid sequence is SEQ ID NO. 32:

FIG. 9-3: mRNA Expression Profile

Quantitative PCR was used to determine relative expression levels of S4T69C3 in mRNA preparation from the body, gut and ovary of adult H. contortus

FIG. 9-4: Homology to known sequences and protein motif scan

FIG. 9-5: SEQ ID No.33

Heterologous expression of S4T69C3 is undertaken in E. coli using pDEST17 (Invitrogen) as the vector. The predicted recombinant protein is 441 amino acids, including the initiation Methionine, His tag, and additional linker residues from pDEST17 vector sequence (shown in bold). The C69C3-5.5 sequence is 419 amino acids, minus predicted signal sequence. The corresponding amino acid sequence is SEQ ID NO. 33.

FIG. 10: Clone YAd189

FIG. 10-1: SEQ ID No.34

From a young adult H. contortus cDNA. An adult gut H. contortus cDNA library is screened using a radiolabelled YAd189 fragment. The screening procedure is repeated to obtain well-isolated positive plaques (ie primary and secondary screens). A number of positive cDNA clones hybridised to the probe; these are excised into pTriplEx (Stratagene) for sequence verification. The vector sequencing primer sites are used to sequence the pTriplEx cDNA library clone. The flanking vector primers T7 (SEQ ID NO.3, GTAATACGACTCACTATAG) and ΔTriplEx5′ (SEQ ID NO.15, TCCGAGATCTGGACGAGC) are used. Clone YAd189#7.2 is found to contain: a ˜1500 by cDNA insert, including start and stop codons (shown in bold/italics and bold respectively). The sequencing primer binds close to the cloning site so the complete sequence of this clone is not available (missing ˜20-40 nucleotides at the 5′ end). Sequence for the YAd189 probe is shown underlined. The corresponding nucleotide sequence is SEQ ID NO. 34 (5′ to 3′).

FIG. 10-2: SEQ ID No.35

An open reading frame (ORF) of 421 aa. First codon in expression clone is boxed. The nucleotide and corresponding amino acid sequence is SEQ ID NO. 35:

FIG. 10-3: mRNA Expression Profile

Quantitative PCR was used to determine relative expression levels of YAd189 in mRNA preparation from the body, gut and ovary of adult H. contortus

FIG. 10-4: Homology to known sequences and protein motif scan

FIG. 10-5: SEQ ID No.36

Heterologous expression of YAd189 is undertaken in E. coli using pDEST17 (Invitrogen) as the vector. The predicted recombinant protein is 443 amino acids, including the initiation Methionine, His tag, and additional linker residues from pDEST17 vector sequence (shown in bold). The YAd189#7.2 sequence is 421 aa. The corresponding amino acid sequence is SEQ ID NO. 36.

FIG. 11: Clone YAd219

FIG. 11-1: SEQ ID No.37

From a young adult H. contortus cDNA library. The vector sequencing primer sites are used to sequence the pBluescript cDNA library clone. The flanking vector primers 17 (SEQ ID NO.3, GTAATACGACTCACTATAG) and T3 (SEQ ID NO.11, CAATTAACCCTCACTAAAG) are used. Clone YAd189 is found to contain: a 464 by cDNA insert, including start and stop codons (shown in bold/italics and bold respectively), with the following nucleotide sequence:SEQ ID NO. 37 (5′ to 3′):

FIG. 11-2: SEQ ID No. 38

An open reading frame (ORF) of 97 amino acids, including a predicted signal sequence (shown in bold). First codon in expression clone is boxed. The nucleotide and corresponding amino acid sequence:SEQ ID NO. 38. There is no homology to known sequences.

FIG. 11-3: mRNA Expression Profile

Quantitative PCR was used to determine relative expression levels of YAd219 in mRNA preparation from the body, gut and ovary of adult H. contortus

FIG. 11-4: Homology to known sequences and protein motif scan

FIG. 11-5: SEQ ID No.39

Heterologous expression of YAd219 was undertaken in E. coli using pDEST17 (Invitrogen) as the vector. The predicted recombinant protein is 94 amino acids, including the initiation Methionine, His tag, and additional linker residues from pDEST17 vector sequence (shown in bold). The YAd219 sequence is 72 amino acids, minus predicted signal sequence. Predicted recombinant protein sequence is SEQ ID No.39.

FIG. 12: Clone S4T55C6

FIG. 12-1: SEQ ID No. 40

mRNA is isolated from eL4bf (tester) and xL3 (driver) parasitic preparations. The mRNA preparations are subjected to the subtractive suppressive hybridisation method. Unhybridised cDNA molecules are cloned into pGEMT-Easy (Promega). This is termed the S4 subtracted cDNA library (eL4bf minus xL3). The S4 subtracted library is propagated using JM109 E. coli cells. Individual colonies are selected, grown in LB-broth under the selection of ampicillin, and plasmid DNA extracted using plasmid DNA prep columns (Qiagen). The plasmid DNA is checked for purity and the presence of a cDNA insert using a spectrophotometer and restriction endonuclease digestion, respectively. A H. contortus cDNA library is screened using S4T55C6 as a probe. The screening procedure is repeated to obtain well-isolated positive plaques (ie primary and secondary screens). A number of positive cDNA clones hybridise to the probe; these are excised into pBluescript (Stratagene) for sequence verification.A contig is assembled using ClustalW (http://www.ch.embnet.org/software/ClustalW.html) from the sequence obtained from the cDNA clones isolated from the library screen. The vector sequencing primer sites are used to sequence the pBluescript cDNA library clones. The flanking vector primers T7 (SEQ ID NO.3, GTAATACGACTCACTATAG) and T3 (SEQ ID NO.11, CAATTAACCCTCACTAAAG) are used. The contig sequence for S4T55C6 cDNA clones is found to contain a 2036 by cDNA insert, including start and stop codon (shown in bold/italics and bold respectively), sequence for the S4T55C6 probe is shown underlined, with the following nucleotide sequence: is SEQ ID NO. 40 (5′ to 3′).

FIG. 12-2: SEQ ID No. 41

An ORF of 320 aa, including a predicted signal sequence (shown in bold). First codon in expression clone is boxed. The nucleotide and corresponding aa sequence is SEQ ID NO. 41 There is no significant homology with known sequences.

FIG. 12-3: mRNA Expression Profile

Quantitative PCR was used to determine relative expression levels of S4T55C6 in mRNA preparation from the body, gut and ovary of adult H. contortus

FIG. 12-4: Homology to known sequences and protein motif scan

FIG. 12-5: SEQ ID No.42

Heterologous expression of S4T55C6 is undertaken in E. coli using pDEST17 (Invitrogen) as the vector. The predicted recombinant protein is 312 aa, including the initiation Methionine, His tag, and additional linker residues from pDEST17 vector sequence (shown in bold). The S4T55C6 sequence is 290 aa, minus predicted signal sequence. The corresponding amino acid sequence is SEQ ID NO. 42.

### BIBLIOGRAPHY

Ausubel et al. In Current Protocols in Molecular Biology. Wiley Interscience (15 BN 047150338), 1987.
Nikolaou et al, 2002. HcSTK, a Caenorhabditis elegans PAR-1 homologue from the parasitic nematode, H. contortus. Int. J. Parasitol. 32, 749-758.
Nikolaou et al, 2004. Genomic organization and expression analysis for hcstk, a serine/threonine protein kinase gene of H. contortus, and comparison with Caenorhabditis elegans par-1. Gene 343, 313-322.
Sambrook et al., Molecular Cloning: A Laboratory Manual. Second Edition. Cold Spring Harbor Laboratories, Cold Spring Harbor, N.Y., 1989.
Sambrook and Russell, Molecular Cloning: A Laboratory Manual. Third Edition. Cold Spring Harbor Laboratories, Cold Spring Harbor, N.Y., 2001.

1. An isolated polynucleotide sequence encoding a H. contortus polypeptide selected from the group consisting of:
(a) a nucleotide sequence of SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. 25 (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2);
(b) a fragment of a nucleotide sequence of SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. 25 (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2); or
(c) a substantially homologous sequence to a nucleotide sequence of SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. 25 (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2).

2. An isolation polynucleotide sequence according to claim 1 wherein said sequence is selected from the group consisting of: the open reading frame (ORF) of nucleotide sequence of SEQ ID No. 5 (FIG. 1-1, FIG. 1-2), SEQ ID No. 8 (FIG. 2-1, FIG. 2-2), SEQ ID No. 12 (FIG. 3-1, FIG. 3-2), SEQ ID No. 16 (FIG. 4-1, FIG. 4-2), SEQ ID No. 19 (FIG. 5-1, FIG. 5-2), SEQ ID No. 22 (FIG. 6-1, FIG. 6-2), SEQ ID No. 25 (FIG. 7-1, FIG. 7-2), SEQ ID No. 28 (FIG. 8-1, FIG. 8-2), SEQ ID No. 31 (FIG. 9-1, FIG. 9-2), SEQ ID No. 34 (FIG. 10-1, FIG. 10-2), SEQ ID No. 37 (FIG. 11-1, FIG. 11-2), SEQ ID No. 40 (FIG. 12-1, FIG. 12-2).

3. An isolated polynucleotide sequence according to claim 1 or claim 2 wherein said sequence at least 80% homologous, preferably 90%, more preferably 95% or most preferably 99% homology to the sequences of claim 1 or claim 2.

4. An isolated polynucleotide sequence which hybridizes under high stringency conditions to an isolated sequence of any one of claims 1-3.

5. An isolated polypeptide sequence of H. contortus comprising the amino acid sequence selected from the group of:
(a) SEQ ID No. 6 (FIG. 1-2), SEQ ID No. 7 (FIG. 1-5), SEQ ID No. 9 (FIG. 2-2), SEQ ID No. 10 (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. 30 (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5);
(b) a fragment of SEQ ID No. 6 (FIG. 1-2), SEQ ID No. 7 (FIG. 1-5), SEQ ID No. 9 (FIG. 2-2), SEQ ID No. (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5); or
(c) a substantially homologous sequence to SEQ ID No. 6 (FIG. 1-2), SEQ ID No. 7 (FIG. 1-5), SEQ ID No. 9 (FIG. 2-2), SEQ ID No. 10 (FIG. 2-5), SEQ ID No. 13 (FIG. 3-2), SEQ ID No. 14 (FIG. 3-5), SEQ ID No. 17 (FIG. 4-2), SEQ ID No. 18 (FIG. 4-5), SEQ ID No. 20 (FIG. 5-2), SEQ ID No. 21 (FIG. 5-5), SEQ ID No. 23 (FIG. 6-2), SEQ ID No. 24 (FIG. 6-5), SEQ ID No. 26 (FIG. 7-2), SEQ ID No. 27 (FIG. 7-5), SEQ ID No. 29 (FIG. 8-2), SEQ ID No. 30 (FIG. 8-5), SEQ ID No. 32 (FIG. 9-2), SEQ ID No. 33 (FIG. 9-5), SEQ ID No. 35 (FIG. 10-2), SEQ ID No. 36 (FIG. 10-5), SEQ ID No. 38 (FIG. 11-2), SEQ ID No. 39 (FIG. 11-5), SEQ ID No. 41 (FIG. 12-2), SEQ ID No. 42 (FIG. 12-5).

6. An isolated polypeptide sequence according to claim 5 wherein said sequence is selected from the group consisting of: SEQ ID No. 7 (FIG. 1-5), SEQ ID No. 10 (FIG. 2-5), SEQ ID No. 14 (FIG. 3-5), SEQ ID No. 18 (FIG. 4-5), SEQ ID No. 21 (FIG. 5-5), SEQ ID No. 24 (FIG. 6-5), SEQ ID No. 27 (FIG. 7-5), SEQ ID No. 30 (FIG. 8-5), SEQ ID No. 33 (FIG. 9-5), SEQ ID No. 36 (FIG. 10-5), SEQ ID No. 39 (FIG. 11-5), SEQ ID No. 42 (FIG. 12-5).

7. An isolated polypeptide sequence according to claim 5 or claim 6 wherein said sequence is at least 80% homologous, preferably 90%, more preferably 95% or most preferably 99% homology to the polypeptide sequences of claim 5 or claim 6.

8. An expression vector comprising a polynucleotide sequence according to any one of claims 1-4 operably linked to a control sequence which is capable of providing for the expression of the polynucleotide sequence by a host cell.

9. A host cell comprising an isolated polypeptide sequence according to any one of claims 5-7.

10. An antibody which binds to a polypeptide sequence according to any one of claims 5-7 encoding a H. contortus protein which is an immunogenic protein.

11. Use of a polypeptide sequence according to any one of claims 5-7 encoding a H. contortus protein for the manufacturing of an immunogenic composition for prophylaxis or treatment of H. contortus infection.

12. An immunogenic composition for the prophylaxis or treatment of H. contortus infection comprising a polypeptide sequence according to any one of claims 5-7 and a pharmaceutically acceptable carrier.

13. An immunogenic composition according to claim 12 further comprising an adjuvant.

14. An immunogenic composition according to claim 12 or claim 13 comprising at least one additional immunogenic sequence derived from another trichostronglyid other than H. contortus.

15. An immunogenic composition according to any one of claims 12-14 comprising antibodies against a protein with a polypeptide sequence according to any one of claims 5-7.

16. Method for the preparation of an immunogenic composition according to any one of claims 12-15 said method comprising the admixing of a protein with a polypeptide sequence according to any one of claims 5-7 and a pharmaceutically acceptable carrier.

17. Method for the preparation of an immunogenic composition according to any one of claims 12-15 said method comprising the admixing of antibodies against a protein with a polypeptide sequence according to any one of claims 6-9 and a pharmaceutically acceptable carrier.

18. Diagnostic kit for the detection of H. contortus disease characterised in that said kit comprises an antibody which bind to a H. contortus protein with a polypeptide sequence according to any one of claims 5-7.

19. Use of a polynucleotide sequence according to any one of claims 1—for the manufacturing of an immunogenic composition for prophylaxis or treatment of H. contortus infection.

20. An immunogenic composition for the prophylaxis or treatment of H. contortus infection comprising a polynucleotide sequence according to any one of claims 1-4 and a pharmaceutically acceptable carrier.

21. An immunogenic composition according to claim 20 further comprising an adjuvant.

22. An immunogenic composition according to claim 20 or claim 21 comprising at least one additional polynucleotide sequence derived from another trichostronglyid other than H. contortus.

23. Method for the preparation of an immunogenic composition according to any one of claims 20-22 said method comprising the admixing a polynucleotide sequence according to any one of claims 1-4 and a pharmaceutically acceptable carrier.

24. Method of prophylaxis or treatment of an animal against H. contortus infection comprising administering a therapeutically effective amount of the immunogenic composition of any one of claims 12-15 or claims 21-23 to said animal.

25. Use of a polypeptide according to any one of claims 5-7 in a method of screening to identify analogous proteins from other helminths, including trematodes, cestodes, nematodes and acathocephala.


SEQUENCE LISTING (42 sequences)

SEQ ID NO: 1 - DNA, 30 bp, Artificial
  His-linker forward
gtcccaccatcaccatcaccataccatggg

SEQ ID NO: 2 - DNA, 31 bp, Artificial
  His-linker reverse
aattcccatggtatggtgatggtgatggtgg

SEQ ID NO: 3 - DNA, 19 bp, Artificial
  T7 Primer
gtaatacgactcactatag

SEQ ID NO: 4 - DNA, 18 bp, Artificial
  SP6 Primer
atttaggtgacactatag

SEQ ID NO: 5 - DNA, 573 bp, Haemonchus contortus
tcgagcggccgcccgggcaggtgactgggccctgcattggtggcctatgtccgtcatctgcagatcagtgcattaccaccgaacaaggacaagtttgttgcgacaaaacgcagattgggacagtgggaacaaaaaccacacccagcacccaaacgagttgcgaggatatgttaaatccacgaacgggtaaatccgattgtccacaatataaatggtattgtaataatagcaaatactacgatttaatgacgcaacagtgtccgaaatcctgcaacagatgtaaagaggcggccgcgacaccagaaggttgtcgggaccttgtgaatcccgtcactggtcgatcgaattgcccaaaattgattgcatactgcaataacagcagctacaggaagctaatggaacaacagtgccccaaaacatgcagattttgtaattaatagatgctcattcgagggatgctccgaaagtgaacaaagattgattagtatagttatcatgataaaacatattttataggcaaaaaaaaaaaaaaaaaaaaaaaaaaagcttgtacacctcggccgcgaccacgct

SEQ ID NO: 6 - protein, 64 aa, Haemonchus contortus
ERPPGQVTGPCIGGLCPSSADQCITTEQGQVCCDKTQIGTVGTKTTPSTQTSCEDMLNPRTGKS

SEQ ID NO: 7 - protein, 161 aa, Haemonchus contortus
MSYYHHHHHHLESTSLYKKAGLQVTGPCIGGLCPSSADQCITTEQGQVCCDKTQIGTVGTKTTPSTQTSCEDMLNPRTGKSDCPQYKWYCNNSKYYDLMTQQCPKSCNRCKEAAATPEGCRDLVNPVTGRSNCPKLIAYCNNSSYRKLMEQQCPKTCRFCN

SEQ ID NO: 8 - DNA, 977 bp, Haemonchus contortus
cttcattttatcatggtctcagccaccatagacccacaaaatcacaacggcgccgttgtcgccgtcatcggatgtggtttgatgggcagcggcattgcacaggtgtgccttgcctctggctacagtgtcaatctatatggaagaagtgatcagacacgggtagcggctaaggaccaaatacggcgtggtttggttaagaacgcctcaaagaaacagggcgtcgagatttcacctgtggacacagtggaaaaagcagtcgattgccaaatgggacagctcaagctttgtgcagatattcgtgaagctgtatcaaacgccttgtacattattgaagcggttgtcgaaaagaaagaagtcaaagataaaatcttcactgaagctcagaatcactgtccacaaggcgctcttctagtcacaaacacatcatcaattagtctagtcgatctgctgccaacaatccgtgatcattcccgctttgccggcttacacttcttcaatccagtgccagtaatgaagctggtcgaggtggtcagcacaccggaaacatcgaaagaaacccagcagaaactaatggatttctgcaaatccttgggaaaaaagccagtttcatgcaaggatacaccaggattcatcgttaatcgcttgctcattccctaccttctcgacagtatccgaatgctcgagcgaggagacgctacgaaggaagatatcgacgatgctctccgttacggtacatcgtgtccaatgggcccaatcgcattgtgtgattacgtcggtctcgacactctctcaaacgtgatgacagtatggagagaatcaatgcctgatgacacgcgatttgcaccaatcccactactggaaaggctcgtccacgagggaaaactcggccgaaagacaaaagaaggcttctataaatactgactacgcgtaatatcgtccactgcataaaattctttttgttaaaaaaaaacctcagc

SEQ ID NO: 9 - protein, 302 aa, Haemonchus contortus
MVSATIDPQNHNGAVVAVIGCGLMGSGIAQVCLASGYSVNLYGRSDQTRVAAKDQIRRGLVKNASKKQGVEISPVDTVEKAVDCQMGQLKLCADIREAVSNALYIIEAVVEKKEVKDKIFTEAQNHCPQGALLVTNTSSISLVDLLPTIRDHSRFAGLHFFNPVPVMKLVEVVSTPETSKETQQKLMDFCKSLGKKPVSCKDTPGFIVNRLLIPYLLDSIRMLERGDATKEDIDDALRYGTSCPMGPIALCDYVGLDTLSNVMTVWRESMPDDTRFAPIPLLERLVHEGKLGRKTKEGFYKY

SEQ ID NO: 10 - protein, 287 aa, Haemonchus contortus
MSYYHHHHHHLESTSLYKKAGFSVNLYGRSDQTRVAAKDQIRRGLVKNASKKQGVEISPVDTVEKAVDCQMGQLKLCADIRETVSNALYIIEAVVEKKEVKEEIFTEAQEHCPPGALLVTNTSSLSLVDLLPTIRDHSRFAGLHFFNPVPVMKLVEVVSTPETSKETQQKLMDFCKSLGKKPVSCKDTPGFIVNRLLIPYLLDSIRMLERGDATKEDIDDALRYGTSCPMGPIALCDYVGLDTLSNVMTVWRESMPDDTRFAPIPLLERLVHEGKLGRKTKEGFYKY

SEQ ID NO: 11 - DNA, 19 bp, Artificial
  T3 Primer
caattaaccctcactaaag

SEQ ID NO: 12 - DNA, 846 bp, Haemonchus contortus
cggcgactcgttgcctgcctggcttcgattcctctactctattcggctccgtccgttccctcagagcctgtcgtcggcgcggcatccgttcatcctccacagccccttgtcggatcattatggaatcttcggcaagtgggcgaatgtactcttcattacagtcctatatgttacaacaattacggttgttggtgtggaattggaggatcacatgagccgatcgacgaaattgacaaatgctgtatgcttcacgataagtgctatgatgctgcagttgataacaagatatgcctggacgtggcatgggagtacatagatatctacaactggcagtgcactaacaatactgctacttgcctcgatgccgaaggaacatgtaaaagagctttgtgtgactgtgatactgcagtagtcaattgctgggcacagcaccctaaaccgaatcgaaaattaaaatgcaatcgttcgaggcttgcaccgacgtcgatatatttccagcattgaaaataccccttcatcgaaattatttcttgcctatacaatacgttagttgtcccttttacgtttgaacttcatcagaaaacacttttaatgctagaacttagcataatgatgctttgtacagacgcctttatagtttttggtaaaattttacacttaactacgtccttatcatcatgtttattctttgtttgaagctgtatttgaataagcgatagtcttagagaaaagagactccattcccaggaaatagccgttacgatgtttgtacatttaatgtcataccgttccccattgtttccgaaaccaggtcaagaaatacttcgaaaaaaaaaaaaaaaaccg

SEQ ID NO: 13 - protein, 167 aa, Haemonchus contortus
RRLVACLASIPLLYSAPSVPSEPVVGAASVHPPQPLVGSLWNLRQVGECTLHYSPICYNNYGCWCGIGGSHEPIDEIDKCCMLHDKCYDAAVDNKICLDVAWEYIDIYNWQCTNNTATCLDAEGTCKRALCDCDTAVVNCWAQHPKPNRKLKCNRSRLAPTSIYFQH

SEQ ID NO: 14 - protein, 168 aa, Haemonchus contortus
MSYYHHHHHHLESTSLYKKAGLEPVVGAASVHPPQPLVGSLWNLRQVGECTLHYSPICYNNYGCWCGIGGSHEPIDEIDKCCMLHDKCYDAAVDNKICLDVAWEYIDIYNWQCTNNTATCLDAEGTCKRALCDCDTAVVNCWAQHPKPNRKLKCNRSRLAPTSIYFQH

SEQ ID NO: 15 - DNA, 18 bp, Artificial
  Lambda Triplex 5′ Primer
tccgagatctggacgagc

SEQ ID NO: 16 - DNA, 1788 bp, Haemonchus contortus
cggatgtgggtcagtcgatcctaagatactggcttatgccttgtatcatcgagagggaaaagtctcttttctctcggatatcaaaagggaatagggttaatattccctaacgcagacacagagattgtggctcgttgagtcacttgcgcggtaacgcattcgaacttggttacgtcgctcagagaccgagctagagttctcttctctagttaaggaacggactccctggaatcggttcagccgtagatagggacgttgtttccgtaaagcatcgcggtttctgtgatgtctcgtgttctctggacggcccttgaaaaaccaaggaaggctattaatctgtgctgcatcgtaccgatatccgcatcaggtctccaaggtgaacagcctctagtcgatagaataatgtaggtaagggaagtcggcaaattagatccgtaacttcgggaaaaggattggctccagtgaccgggtcggttggcctgttaactgatgcttgtgcggtttcgtccgtggctggctgatgctcacgctgatgctggactagtgacaagactgccttgtggacgttttcagtgtgtgctttgttttcggacttagtatcctgaccgaccatcaaaacggtcgaactggaactggtacggactcggggaatccgactgtctaattaaaacagaggagacagatggtccttgcggacgtttactgtctctgatttctgcccagtgctctgaatgttaaatcgtagtaattcgagtaagcgcgggtaaacggcgggagtaactatgactctcttaaggccggaattccgggcccctcctacttttgctgacctcggaaaatctgcgaaggatctattcaacaaaggatacaatcatggttttattaaagtcgataccaccacgaaagctggtgagaataaagaggtagaattcaaaacatctgctgctcataatctcggctctggcaaactcggaggaaaccttgatgtcaaatacaagatcccttcctacggtctgactctcacggagaaatggaacactgagaaccaactcggaacaataattgaagttaatgagcagtttggtcgaggactcaaactcacttttgattctctttatgctcctcacgctggaaagaggaccggcaaactgaaggctgattggtctctcaacactgccagaatcactggtgaagttggcatcaccgccgctccagttatcaattgtgcaggtgtttttggtcgtgatgggtggctcttcggtgcttcggccacattcgatacctcttcaaacaaggtgtcaacgaccgcattggccttcgggcatcaaacctctcactacactctacactcatttgttgtgaattcaaccgactttggtgcttcgctcttccacagagtcagccccaacgttgaacttggtactatgcttggctggaaggttgggggtaatggtgctgatttcgctgttgctgcaaaatatagtcctaccagagatctgacgcttcgtggtaaagttaacaatagcagccaggtggcattggctgtcacacattcgctctcgcctgacctcaaagctacattgtcgagtcagttcaatctttcctcgagtgatgttcataagttcggaatgggtcttgaatatgagccactgtaatatctttgtgtgttgaccgatcattttcattagatctagattttttatagtctagtgcaatgatagtctgttggtccctttgaagttgtttcttgtcaaacctattaaagttgcgtaatgaatatttatgccg

SEQ ID NO: 17 - protein, 290 aa, Haemonchus contortus
MTLLRPEFRAPPTFADLGKSAKDLFNKGYNHGFIKVDTTTKAGENKEVEFKTSAAHNLGSGKLGGNLDVKYKIPSYGLTLTEKWNTENQLGTIIEVNEQFGRGLKLTFDSLYAPHAGKRTGKLKADWSLNTARITGEVGITAAPVINCAGVFGRDGWLFGASATFDTSSNKVSTTALAFGHQTSHYTLHSFVVNSTDFGASLFHRVSPNVELGTMLGWKVGGNGADFAVAAKYSPTRDLTLRGKVNNSSQVALAVTHSLSPDLKATLSSQFNLSSSDVHKFGMGLEYEPL

SEQ ID NO: 18 - protein, 312 aa, Haemonchus contortus
MSYYHHHHHHLESTSLYKKAGFMTLLRPEFRAPPTFADLGKSAKDLFNKGYNHGFIKVDTTTKAGENKEVEFKTSAAHNLGSGKLGGNLDVKYKIPSYGLTLTEKWNTENQLGTIIEVNEQFGRGLKLTFDSLYAPHAGKRTGKLKADWSLNTARITGEVGITAAPVINCAGVFGRDGWLFGASATFDTSSNKVSTTALAFGHQTSHYTLHSFVVNSTDFGASLFHRVSPNVELGTMLGWKVGGNGADFAVAAKYSPTRDLTLRGKVNNSSQVALAVTHSLSPDLKATLSSQFNLSSSDVHKFGMGLEYEPL

SEQ ID NO: 19 - DNA, 881 bp, Haemonchus contortus
cggccggaattccgggtcgtgctctgtgttttgtgtggaatcgcttttgcagcacccagacaaaagcgcctcactgtgggcacgatctccgttactggaggagtcggtggttccacaggatgtgtagtaactggtaatgtgctttatgcaaatggtttccgcctacgtgagctcacttcatcggaacaacaggaattggtaaactatgagaaacaggtggctgattataaagaggctgtgaagaaggctctcaaggagcgtcaggaaagcctaaaagcccgtatggctggtaagaaggagaaagccgtgacaccaaaggaggaggatcttcctaaagcaccagagaagccatcattctgcaccgaagatgacactacacaattctacttcgatggatgcatggttcagggcaacaaggtctacgttggcaacacattcgcccgtgatctggatcacaacgaaattcaagagctgaaagaattcgagaaaaaacagacggtctaccaagaatacgtccagaagcagatccaacagcaagtaaccaacctatttggtggtgctgacttcttctcatccttcttcaatggcgcatcaaaggatgctaccaccaccacggtagctccagctcttcccgatgatgctcccgaacaaccacctgtgcctaacttctgtacgagaatctattgaatgcctgtgggagaacagtgttaagtgaatgcaatagtaaacagttgctgtacttcatacagctacttctctattcacggtgtcgactggtgttttagtcattttatttgcaatattttgcagtgtttctgtattcgttgtacatgtcaataatgaattttaatatcacaaaaaaaaaaaaaaaaaaaactgca

SEQ ID NO: 20 - protein, 228 aa, Haemonchus contortus
RPEFRVVLCVLCGIAFAAPRQKRLTVGTISVTGGVGGSTGCVVTGNVLYANGFRLRELTSSEQQELVNYEKQVADYKEAVKKALKERQESLKARMAGKKEKAVTPKEEDLPKAPEKPSFCTEDDTTQFYFDGCMVQGNKVYVGNTFARDLDHNEIQELKEFEKKQTVYQEYVQKQIQQQVTNLFGGADFFSSFFNGASKDATTTTVAPALPDDAPEQPPVPNFCTRIY

SEQ ID NO: 21 - protein, 218 aa, Haemonchus contortus
MSYYHHHHHHLESTSLYKKAGLGGVGGSTGCVVTGNVLYANGFRLRELTSSEQQELVNYEKQVADYKEAVKKALKERQESLKARMAGKKEKAVTPKEEDLPKAPEKPSFCTEDDTTQFYFDGCMVQGNKVYVGNTFARDLDHNEIQELKEFEKKQTVYQEYVQKQIQQQVTNLFGGADFFSSFFNGASKDATTTTVAPALPDDAPEQPPVPNFCTRIY

SEQ ID NO: 22 - DNA, 1609 bp, Haemonchus contortus
tgatgaacgaatacagacagacagttgcaaatggaactgcattcagtatgtgtggaactctaccaagcactctatccatgttccagctgacaggggacgattcgcttactcttatggctctcacgactattttcacctgtaacgtaactaatcctactgttcagccaggatattcgtacaacggttatactataagctatttaccgtccggcatggattcaagcacaatattcgatatcgcactcgcccagtgggaagaagagccccttctttatggagttggcgaaaatgtggtgtacagtaacacaaaattgaaggaatgggctaatatggtttactacaagtctactaaagttggatgctattataatttttgcaaccaaacaggaccgccaacacacgcaatcgcttgcttgttcaacatggtaccacaagagaaccagccggtatataatccaactcaaggagttgatggatgtaatttggacgagaactgtaatgaaaagcttagcggatccacttgcgtcaaaacaagtgggaacggaatgtatgcaggcctttgtgatcccgaagggaattcattcagcacatccacgaccacaaccacagtggcaactacagcaggaagttcttcaactctccctactactccagtaaagtcaacgccctcaacaaaagcaacgtctggatcaacgccctctacaaaagcaacgtctggatcaaccccctcaacgaaagcgacgtctggatcaacaccttcaaccaaagcgacgtctggatcaacaccttcaaccgaagcgacgtctggatcaacgccttcaacaaaagcgacgtctggaacagcaacgacgcctggaacaacaatggcatctacagcaacagcgacacccggaccaactacaactccgggcacaccccttactatgacacaggcagtccgcgataagatcctcaatatgcacaactatcgtagatcaagacttgcccaaggattggttccgaatggtacttcaggcaaaaacttacctcaaggaacggatatagctcaaatgcagtacaaccagacgcttgaggatgcggcgcagaaatatgcgaatacctgcccaaaaggaatctcacctgtatcatcacgtgagggcatgggcgaaatttatcagtctcttccatccaataccttgacattaatggacgctatagaaatggcattaaaagcatcctggcatccaatcatggtgattggctttgacccgaatttggaatttacagatacgactctacatttatctaaagcgccacttatgttcactcaaatggcatggggtgctacttatcaagtcggctgtggtgctactcgctgttcggataacactaccgttatttgtcgctataacccaagtggcaacattcccggtcaaactctctacaccgcaggttcaacgtgctcagcatgtccgagtgactgtacaacagcttacttgtacaaagggctctgcatatttagtagtagctaaactatcttgtcttcgttcttaccaaccttctagagtcaattcttcgaataaagtcgaatttaaaaaaaaaaaaaaaaaaaaaaaaaaaaaaaaaaaaa

SEQ ID NO: 23 - protein, 502 aa, Haemonchus contortus
MNEYRQTVANGTAFSMCGTLPSTLSMFQLTGDDSLTLMALTTIFTCNVTNPTVQPGYSYNGYTISYLPSGMDSSTIFDIALAQWEEEPLLYGVGENVVYSNTKLKEWANMVYYKSTKVGCYYNFCNQTGPPTHAIACLFNMVPQENQPVYNPTQGVDGCNLDENCNEKLSGSTCVKTSGNGMYAGLCDPEGNSFSTSTTTTTVATTAGSSSTLPTTPVKSTPSTKATSGSTPSTKATSGSTPSTKATSGSTPSTKATSGSTPSTEATSGSTPSTKATSGTATTPGTTMASTATATPGPTTTPGTPLTMTQAVRDKILNMHNYRRSRLAQGLVPNGTSGKNLPQGTDIAQMQYNQTLEDAAQKYANTCPKGISPVSSREGMGEIYQSLPSNTLTLMDAIEMALKASWHPIMVIGFDPNLEFTDTTLHLSKAPLMFTQMAWGATYQVGCGATRCSDNTTVICRYNPSGNIPGQTLYTAGSTCSACPSDCTTAYLYKGLCIFSSS

SEQ ID NO: 24 - protein, 594 aa, Haemonchus contortus
MLLVNQSHQGFNKEHTSKMVSAIVLYVLLAAAAHSAFAADLGSRSHHHHHHTMITSLYKKAGFMNEYRQTVANGTAFSMCGTLPSTLSMFQLTGDDSLTLMALTTIFTCNVTNPTVQPGYSYNGYTISYLPSGMDSSTIFDIALAQWEEEPLLYGVGENVVYSNTKLKEWANMVYYKSTKVGCYYNFCNQTGPPTHAIACLFNMVPQENQPVYNPTQGVDGCNLDENCNEKLSGSTCVKTSGNGMYAGLCDPEGNSFSTSTTTTTVATTAGSSSTLPTTPVKSTPSTKATSGSTPSTKATSGSTPSTKATSGSTPSTKATSGSTPSTEATSGSTPSTKATSGTATTPGTTMASTATATPGPTTTPGTPLTMTQAVRDKILNMHNYRRSRLAQGLVPNGTSGKNLPQGTDIAQMQYNQTLEDAAQKYANTCPKGISPVSSREGMGEIYQSLPSNTLTLMDAIEMALKASWHPIMVIGFDPNLEFTDTTLHLSKAPLMFTQMAWGATYQVGCGATRCSDNTTVICRYNPSGNIPGQTLYTAGSTCSACPSDCTTAYLYKVVIACREVLEDHNQPYHICRGFTCFKKPPTPPPEPET

SEQ ID NO: 25 - DNA, 1198 bp, Haemonchus contortus
cgggtcgaaacatggaatggcaagaattcagtcgatcactcctggtggtccagtgacaatgcgagattgattcgcggcacggaaggcatgttgttcccaccgtttctgaagaaatctgacagactctacgtcttcattgcccaactatgcagatcaatatggctcgaatttcaaaaagaagtggaatacgagggagtaccggcttatcggttcattttaccgcctgccgtcttcgatccgaaaatccctgaaaatgatggctactgcaatcctactgacaagaagttctttgaatcacagaacgaaaccgatgactgtttcccggaaggattgcttgaaatcagcaaatgtctgcgaagtcaacctccgatcatgatctcattaccaaacttcaactttgctcctgaggaagtgcgccaatccgtaaaaggactgaacagcaccgatcctgagcgggacattattctcgtagatattgaacctcggttgggtgcggtacttcgtgctcatcgtcgtttccaagtgaatattgaaatgtggaaaggaaaggatcttgtgtttccagtgaacctcaacaagactcgatcatcgttgattccggttctgattatccacgaagatgctactatcgaccgcgaaactctgaacacgattaaaagcgaattgattcaaaccgaatggtgggcgcattccatcgccactgcaatggctggagcaggattggctctgattgccatcgctgctatctatgccctattaaagtccaaattacttcgtcgaggcaaatccgactctgtttcaccgttaccggcttacgaaccacgagtatcatcgagcggcaaaggaagctatacatagaatatatcgattaattgattgcctgaccaacggacagttggtcgtcagttcgggcgtgattttcctttttttcttgaaattcacccatctaactcccaactaaatcagttttcccatgtgactttaacccaacactaattcaacaacaaaacggtgtcaggctttatgttttgttaaatgagttattgattgatttgtagccgttgttaggtttgaggttgaatctctgattgtttttttttcatgacctagttggtatactgtaattttctcaatttcccgaaagtacggtgatcagtacaaaatcctcgtgtagttcgttgtacactctctccggaat

SEQ ID NO: 26 - protein, 289 aa, Haemonchus contortus
RVETWNGKNSVDHSWWSSDNARLIRGTEGMLFPPFLKKSDRLYVFIAQLCRSIWLEFQKEVEYEGVPAYRFILPPAVFDPKIPENDGYCNPTDKKFFESQNETDDCFPEGLLEISKCLRSQPPIMISLPNFNFAPEEVRQSVKGLNSTDPERDIILVDIEPRLGAVLRAHRRFQVNIEMWKGKDLVFPLVFPVNLNKTRSSLIPVLIIHEDATIDRETLNTIKSELIQTEWWAHSIATAMAGAGLALIAIAAIYALLKSKLLRRGKSDSVSPLPAYEPRVSSSGKGSYT

SEQ ID NO: 27 - protein, 252 aa, Haemonchus contortus
MSYYHHHHHHLESTSLYKKAGFRVETWNGKNSVDHSWWSSDNARLIRGTEGMLFPPFLKKSDRLYVFIAQLCRSIWLEFQKEVEYEGVPAYRFILPPAVFDPKIPENDGYCNPTDKKFFESQNETDDCFPEGLLEISKCLRSQPPIMISLPNFNFAPEEVRQSVKGLNSTDPERDIILVDIEPRLGAVLRAHRRFQVNIEMWKGKDLVFPVNLNKTRSSLIPVLIIHEDATIDRETLNTIKSELIQTEWWAH

SEQ ID NO: 28 - DNA, 579 bp, Haemonchus contortus
cggcgcgtacatcaacacgatgacagaagcgtcaccggctattaaacgagccgctattgttggaagtgctgatggctctatttgggctcggactcaggatccaaacacattcgctgctactgatgctgagttgaagaaattcgttgggctctttgataacctgaatgatgttccagcacaaggtgtcgatctcgaagacgttcactacattgtgccacgcactgaagaaaacctcatctttggcaagaaagataagagtggatttttcgcggcaaaaactaagtctgcagtgctgatcgctgtctatgagggagagaaccaggtgtcagctcaagtacgtgcatccgttgagaagctagccaagtatcttgaggagatcggctactaggccgccttgtaatgatctgccttgatggttagccaaccatcgtccttcatgcgtgttgtcttcaaaattccatcagcttcactccaccaataccattccttattcgagtaatttttaaaatgctagattgaattgcggctcatattgtcttcctcgcacgtcatctaattttgcatgctatactcgccccg

SEQ ID NO: 29 - protein, 122 aa, Haemonchus contortus
MTEASPAIKRAAIVGSADGSIWARTQDPNTFAATDAELKKFVGLFDNLNDVPAQGVDLEDVHYIVPRTEENLIFGKKDKSGFFAAKTKSAVLIAVYEGENQVSAQVRASVEKLAKYLEEIGY

SEQ ID NO: 30 - protein, 144 aa, Haemonchus contortus
MSYYHHHHHHLESTSLYKKAGFMTEASPAIKRAAIVGSADGSIWARTQDPNTFAATDAELKKFVGLFDNLNDVPAQGVDLEDVHYIVPRTEENLIFGKKDKSGFFAAKTKSAVLIAVYEGENQVSAQVRASVEKLAKYLEEIGY

SEQ ID NO: 31 - DNA, 1383 bp, Haemonchus contortus
gcgcttgttacgatcagcctactagcttcggtggccattttggcccttcctcttggccgcaatttgccggaaggagagctgtatgcactactggtggctggttccaagggatggtacaattatcgacatcaggctaatgttgcccacgcgtaccacgtcctgctagaacacggagtaccagcaaagaacatcatcgtaatgatgtacgacgacattgccaaccacaaactaaacccgtataaagggaagctcttcaacagacctaatggtcctgacgtttactctggattaaaaattgattacaaaggtgatgctgtcagtgcaggaaactttctagccgtccttaaaggagacagagatgctgtgcagggaggaaatggccgtgtgattgaaagcaacgagaatgaccgaattttcgtatactacactgatcttggagctacaggtctcatggcttttccgactggtatgctcatggcaagacaactgattgaggccttagaagaaatgcacaaaaatcgcaagtacaatcaattggtgttttatctggcagcttgtgagagtggttcaatgttcaaagatcttttaggaaagaagttgaatgtgtacgctgttacctctgctgatgaagacgacttggagtttgccacatattgcgaggacgaactattagaattgccatgtcttggggatgagttctcggtgaactggatggaagattccgacaagcagggcatcagtattgagacgttggatgaacagttcgaagtcgcgaaaggactcacctatcagtctgaagtgtctcattacggtaatctgagcatagccgatgagcctgtaggctggttccatggcttccgcaaagacacttcgaacacaggcaaaaacattacccagtctacttaccaaaggagttcatggtcatcaagagacgtcgaacttctttatctccagaagttgaaacaacttggtcttcgtccagatacaatcgacgatgaaatttttattgctgaagagaatcatcgcccaattggagagtttttctcagtactggtacatcgcatcgtccgtggtcaggatttgagacagaatgtacttagaaagagagccggtgttacaaattatggctgtcatgaagaggtcgtacgagccttctatacgacttgcagtgatttcaacaagcacgactatgcgctgaagtatatgtatgtgctgagcaacctgtgtaccacgctcgctaactctgcgataatcatcaaagcaatgaatgcgacctgccttgaagaccgctcccacttgttttgaaggattcccattccaatgactactcatttctgttcacttcaataaagatttgcaagaaaaaaagtcgacgcggccgcg

SEQ ID NO: 32 - protein, 434 aa, Haemonchus contortus
ALVTISLLASVAILALPLGRNLPEGELYALLVAGSKGWYNYRHQANVAHAYHVLLEHGVPAKNIIVMMYDDIANHKLNPYKGKLFNRPNGPDVYSGLKIDYKGDAVSAGNFLAVLKGDRDAVQGGNGRVIESNENDRIFVYYTDLGATGLMAFPTGMLMARQLIEALEEMHKNRKYNQLVFYLAACESGSMFKDLLGKKLNVYAVTSADEDDLEFATYCEDELLELPCLGDEFSVNWMEDSDKQGISIETLDEQFEVAKGLTYQSEVSHYGNLSIADEPVGWFHGFRKDTSNTGKNITQSTYQRSSWSSRDVELLYLQKLKQLGLRPDTIDDEIFIAEENHRPIGEFFSVLVHRIVRGQDLRQNVLRKRAGVTNYGCHEEVVRAFYTTCSDFNKHDYALKYMYVLSNLCTTLANSAIIIKAMNATCLEDRSHLF

SEQ ID NO: 33 - protein, 434 aa, Haemonchus contortus; identical to SEQ ID NO: 32

SEQ ID NO: 34 - DNA, 1483 bp, Haemonchus contortus
ttcgtgtttctcattcctcaattatttgccgcctccatcaaggagaaggaatcgggggaagtgaatcccttgacagagctccataaggagttggctcaaaaagcagtagatgacatgaatgctaaatcgaatgatttgtatcgatgggacgtctatacaatagatcaagttcatcaacaaatgcataatggtattcaatacaagatgcacttaacactcgtacaaacagactgccggaaaacgggatctgaacttactcgtaccgactgtaaacacacggaccgactgaagaaatgcactgctcaaggtgaacatcgagcttgggagaactacgagaacatcgaaattcggcattgtgagcagccgttcaagcgatcaaaacggatggctaagacatcaaaacactctgtcgaatacaaaagactcagccgacacataaagccaagggactacgttgcttggaaccaattcaccgacttcgtagacaggcatgacaaactctacagcagtaaacgtgaagctctgaagcggttccgaatcttcaagcggaatctgaaggcaattcgtacatggcaagaaaacgaaaagggaacggccgtttatggaatcacccagttctcggatctttcacctgaagagttcaagaagatctacctgccatatacttgggaacagccaatttacccgaaccgaatcgcggatttggccgccgaaggaataagtctggacgacccactgcctgactcattcgattggagaacgcatggcgctgtgacagccgttaaaaaccaaggcaattgcggaagctgctgggcattttcaacaacgggaaatattgagggtcaatggtacctggctaaaaacaagctcttatcactgtcggaacaggaactcgttgattgcgatatcatcgatcaaggatgtaacggtggactgccaagccaggcttacaaagaaataatccgaatgggaggtttggaaccagaagacgcatatccgtacgatggcaagggagagacatgtcacatcgtccgctctgaaattgctgtctatatcaacgactcagtggagctgccacacgacgaggatagcatgaaagcatggctggtgaagaagggaccaatatccattggcatcaacgcgaatccccttcaattttatcgccatggcatttcacatccgtggaaaattctctgcgaaccgtttatgctgaatcatggcgtcctcattgttggatacggtagcgagaagaacaagccgtactggattatcaagaattcatggggaactaaatggggagaggagggatattaccggctctaccgagggaaaaatgtttgcggtgtacacgagatggccacgtcggctcttgtgttgtagtcctggctcatttctacagcaacaactctgactgtcctcaagaatatgtattatactttagtgttgagtttgaagctgtcgttgtttcgctttatcacggccg

SEQ ID NO: 35 - protein, 421 aa, Haemonchus contortus
MNAKSNDLYRWDVYTIDQVHQQMHNGIQYKMHLTLVQTDCRKTGSELTRTDCKHTDRLKKCTAQGEHRAWENYENIEIRHCEQPFKRSKRMAKTSKHSVEYKRLSRHIKPRDYVAWNQFTDFVDRHDKLYSSKREALKRFRIFKRNLKAIRTWQENEKGTAVYGITQFSDLSPEEFKKIYLPYTWEQPIYPNRIADLAAEGISLDDPLPDSFDWRTHGAVTAVKNQGNCGSCWAFSTTGNIEGQWYLAKNKLLSLSEQELVDCDIIDQGCNGGLPSQAYKEIIRMGGLEPEDAYPYDGKGETCHIVRSEIAVYINDSVELPHDEDSMKAWLVKKGPISIGINANPLQFYRHGISHPWKILCEPFMLNHGVLIVGYGSEKNKPYWIIKNSWGTKWGEEGYYRLYRGKNVCGVHEMATSALVL

SEQ ID NO: 36 - protein, 443 aa, Haemonchus contortus
MSYYHHHHHHLESTSLYKKAGFMNAKSNDLYRWDVYTIDQVHQQMHNGIQYKMHLTLVQTDCRKTGSELTRTDCKHTDRLKKCTAQGEHRAWENYENIEIRHCEQPFKRSKRMAKTSKHSVEYKRLSRHIKPRDYVAWNQFTDFVDRHDKLYSSKREALKRFRIFKRNLKAIRTWQENEKGTAVYGITQFSDLSPEEFKKIYLPYTWEQPIYPNRIADLAAEGISLDDPLPDSFDWRTHGAVTAVKNQGNCGSCWAFSTTGNIEGQWYLAKNKLLSLSEQELVDCDIIDQGCNGGLPSQAYKEIIRMGGLEPEDAYPYDGKGETCHIVRSEIAVYINDSVELPHDEDSMKAWLVKKGPISIGINANPLQFYRHGISHPWKILCEPFMLNHGVLIVGYGSEKNKPYWIIKNSWGTKWGEEGYYRLYRGKNVCGVHEMATSALVL

SEQ ID NO: 37 - DNA, 464 bp, Haemonchus contortus
aaacgaccatggctggaggaacatcagcaaccaccactgttgccttactattcgtcgccacagttctggtaacagtcatggccagccgtacagacagatactcaaacaacggcctctttggtttcctcaacagagaaccatcttctcataggcgatttagcaacgatttctacaattccctccgtttatacaaagatcgacgagtaaagaggtttccggtcggtctatattactcgctgatgggtgaggatctgacaaacccgcacaagagaccagattacgacgatgtcgatgtttattaacctggatctgttgtttgggaaggcgactagtataaaaatcgataatcgatggattctacgcagctgtggtgatcgatgctgccttgtgaatagatacgtgtgtcagtccatttttgggaataaagagttttgttgaaaaaaaaaaaaaaaaaaaaaaaaaaa

SEQ ID NO: 38 - protein, 97 aa, Haemonchus contortus
MAGGTSATTTVALLFVATVLVTVMASRTDRYSNNGLFGFLNREPSSHRRFSNDFYNSLRLYKDRRVKRFPVGLYYSLMGEDLTNPHKRPDYDDVDVY

SEQ ID NO: 39 - protein, 94 aa, Haemonchus contortus
MSYYHHHHHHLESTSLYKKAGFSRTDRYSNNGLFGFLNREPSSHRRFSNDFYNSLRLYKDRRVKRFPVGLYYSLMGEDLTNPHKRPDYDDVDVY

SEQ ID NO: 40 - DNA, 2036 bp, Haemonchus contortus
gccattatttcagttcctgaaccaataaatagaaccgaagtcctcttttattattccatgatcgaccattcaagcaaacgcttgttctgagcactctaatttattcaaagtaaactcgctagccactaaagaagccgaaacctcaatagaaaaccagcaaatagttacaacagcgattaacgccaaaggcgaatcactgcatgtaactcagatccaactacgagctttttaaccgcagcaatgacgatttacactagtggagctggaattaccgcggctgctggcaccagacttgccctccacttgatcctcgaaaaaaggtttatgatcaactcattgaaatgaccgttccataggaatggtctttttatttttagtcactatctcatctgttcaagattggataagtttcgcgcctgctgccttccttggatgtggtagccgtttctaaggctccctctccggagtcgaaccttattctccgttacccgtaataaccatggtagactaatagactaccatcaggttgatagggcagacactttttcggaatatcggcgaacaagccgccgatatgctgcgttattcagagtcagcaaactgcacccgaaagtgattgatttgctctaataattgcaccctttcaaaagtcagggtttatttgcatgtattagctccagaattaccgcagttatccaaaaggacgttccgaataaatgacatctgctctaatgagccgttcgcagtttctcttcaaagatgaactcgacatgcatggcttaatctttaaactgagccggaattccgggttttctcaaaatggcatctcctttgagcctgttgctgtttggagtaattgcaggagtattagctgaaaacgaaaattcaaatagccatccattcaataatggagcagtactgcctgatgctatcaaaaagccgctgagcccctatttgaacggcataaacattgagacaaactttcacgaagaactgacagagctgagcaagatcaaccctgtgctatccggagttatggaggagcttcacgaaaatatcattgccatggaatcagttttgaaggctaaatatttatttttcactgagattatagaaatctccaaaaagaagaaagataacattggcagcgatgacccagcttcaatattgggaaaggctttatacagtgttccagaggaacgtcgcgaaattctggctgattttctaaatttcagtgaacgtcttggatatggaacagccttccaaatgccagttcatactaatgtgagcgctgggataaacgatttggcgtcctcggttactccacgaccacgaaacgattcagacgttaaactgagaaagtcactgaaaaccatcgaaactatactaactgcagtcaataattccctacctgacagacttaagaaaaatagttcttgggtccttttggtacaattagttcgtatgacaaaaactaattataaatcactgacatttgaggacatcaaaaacatttttctagctctaagaaacgcaagttttgctttttacaaagatgttagtctacgatttctagaaaacgaggtggattttgcagctacagcattatatgatattcttcaaaagctttgcgatattgcaattaaacttaatcctgatcgtggagaatcgctaaaaccatatttcaattactatttgggcgccaatatgagatataatactagtattactcattatttctccagagttggaaaagttgcttcggcagcggagttctgttaacttcccgtcaaaaagatagctgaacgactttagccatgtatttctgctatgattggtgcttccagataattgaccaatcacagaaacggcctagtacatgcgtctctgtgattggtcgactattcggcagcaccaaccacagccgaaaccgttgagtaaatcgactgtattcatatttttaacgttagaaacatgttgtaatttttgaaagtagcaaaataaagggtcgaattgatttcaaaaaaaaaaaaacc

SEQ ID NO: 41 - protein, 320 aa, Haemonchus contortus
MASPLSLLLFGVIAGVLAENENSNSHPFNNGAVLPDAIKKPLSPYLNGINIETNFHEELTELSKINPVLSGVMEELHENIIAMESVLKAKYLFFTEIIEISKKKKDNIGSDDPASILGKALYSVPEERREILADFLNFSERLGYGTAFQMPVHTNVSAGINDLASSVTPRPRNDSDVKLRKSLKTIETILTAVNNSLPDRLKKNSSWVLLVQLVRMTKTNYKSLTFEDIKNIFLALRNASFAFYKDVSLRFLENEVDFAATALYDILQKLCDIAIKLNPDRGESLKPYFNYYLGANMRYNTSITHYFSRVGKVASAAEFC

SEQ ID NO: 42 - protein, 312 aa, Haemonchus contortus
MSYYHHHHHHLESTSLYKKAGFGAVLPDAIKKPLSPYLNGINIETNFHEELTELSKINPVLSGVMEELHENIIAMESVLKAKYLFFTEIIEISKKKKDNIGSDDPASILGKALYSVPEERREILADFLNFSERLGYGTAFQMPVHTNVSAGINDLASSVTPRPRNDSDVKLRKSLKTIETILTAVNNSLPDRLKKNSSWVLLVQLVRMTKTNYKSLTFEDIKNIFLALRNASFAFYKDVSLRFLENEVDFAATALYDILQKLCDIAIKLNPDRGESLKPYFNYYLGANMRYNTSITHYFSRVGKVASAAEFC